(19) United States
(12) Patent Application Publication
(10) Pub. No.: US 2026/0126425 A1
(43) Pub. Date: May 7, 2026

(54) NANOBODY-FUNCTIONALIZED BIOLOGICAL NANOPORES AND MEANS AND METHODS RELATED THERETO

(71) Applicants: Rijksuniversiteit Groningen, Groningen (NL); Aarhus Universitet, Aarhus (DK)

(72) Inventors: Giovanni MAGLIA, Glimmen (NL); Xialin ZHANG, Boston, MA (US); Jørgen KJEMS, Aarhus (DK)

(21) Appl. No.: 19/224,104

(22) Filed: May 30, 2025

Related U.S. Application Data
(63) Continuation of application No. PCT/NL2023/050633, filed on Dec. 1, 2023.

(30) Foreign Application Priority Data
Dec. 2, 2022 (EP) 22211193.2

(51) Int. Cl. G01N 33/487; G01N 33/569

(52) U.S. Cl. CPC G01N 33/48721; G01N 33/56983; G01N 2333/165

(57) ABSTRACT

The invention relates to means and methods for analysis of analytes using nanopore-based sensors, for example, to methods, nanopore systems and devices for the stochastic detection of (label-free) analytes in complex samples, for example the specific detection of a protein biomarker in a bodily sample. Provided is a method for detecting the presence of at least one analyte in a sample using a nanopore system comprising a cis chamber comprising a first conductive liquid medium in liquid communication with a trans chamber comprising a second conductive liquid medium through a modified nanopore, comprising:
(a) adding a sample to be analyzed for the presence of an analyte to the cis chamber; (b) optionally applying an electrical potential across the modified nanopore; (c) measuring ionic current passing through the modified nanopore, wherein said modified nanopore is a biological nanopore that is functionalized with a 5 to 50 kDa, preferably 10 to 40 kDa, recognition element (e.g., proteinaceous recognition element) R capable of specifically binding to the analyte.

### INCORPORATION BY REFERENCE

This application is a continuation of International Patent Application No. PCT/NL2023/050633, filed Dec. 1, 2023, which claims benefit of European Application No. EP22211193.2, filed Dec. 2, 2022, each of which is incorporated herein in their entirety and by this reference thereto.

### SEQUENCE LISTING

The instant application contains a Sequence Listing which has been submitted electronically in XML file format and is hereby incorporated by reference in its entirety. Said XML copy, created on Aug. 18, 2025, is named 64828-711_301_SL.xml, and is 4,804 bytes in size.

### BACKGROUND

Determining analytes (e.g., target analytes) in a sample is an important aspect of scientific studies. The presence or absence of analytes in a sample can be important for clinical aspects.

### SUMMARY

In an aspect, the present disclosure provides a method for detecting the presence of at least one target analyte in a sample using a nanopore system comprising a cis chamber comprising a first conductive liquid medium in liquid communication with a trans chamber comprising a second conductive liquid medium through a modified nanopore, comprising (a) adding a sample to be analyzed for the presence of a target analyte to the cis chamber, (b) optionally applying an electrical potential across the modified nanopore, and (c) measuring ionic current passing through the modified nanopore, wherein said modified nanopore is a biological nanopore that is functionalized with a 5 to 50 kDa, for example 10 to 40 kDa, proteinaceous recognition element R capable of specifically binding to the target analyte and wherein R dynamically moves in and out of the nanopore to provoke transient current blockage events, and wherein binding of R to the target analyte modulates its dynamic movement, thereby inducing a change in the frequency and/or magnitude of the current blockage events, and wherein the change in the frequency and/or magnitude of current blockage events indicates the presence of the target analyte in the sample.

In some embodiments, the modified nanopore is an oligomeric assembly comprising or consisting of monomers of the general formula N-L-R, wherein N is a monomer of a pore-forming toxin having a largest internal diameter (e.g., internal lumen diameter) of 5 nm to 20 nm, and L is a flexible linker attached to the cis entrance of the pore.

In some embodiments, binding of R to the target analyte increases the time of R staying outside of the pore, thereby decreasing the frequency and/or magnitude of the current blockage events.

In some embodiments of any one of the preceding embodiments, the biological nanopore is functionalized with at least two different proteinaceous recognition elements R′ and R″, for example wherein R′ and R″ bind to distinct sites of the target analyte.

In some embodiments of any one of the preceding embodiments, the target analyte is a protein, protein assembly, protein/DNA assembly, protein/RNA assembly, steroid, lipid, lipid membrane, lipid particle, bacterium, virus capsid, virus particle, cell, dendrimer, polymer, or any combination thereof, wherein the target analyte is a protein, when the protein is selected from the group consisting of a folded/native protein, a clinically relevant protein, biomarker, pathogenic protein, cell surface protein.

In some embodiments, the target analyte is a protein, preferably selected from the group consisting of a folded/native protein, a clinically relevant protein, biomarker, pathogenic protein, or cell surface protein.

In some embodiments of any one of the preceding embodiments, the sample is a complex sample comprising a mixture of proteins, wherein the sample comprises a clinical sample, such as a bodily fluid, such as whole blood, plasma, urine, feces, saliva, cerebrospinal fluid, breast milk and sputum.

In an aspect, the present disclosure provides a modified proteinaceous nanopore having a minimal pore diameter of 5 nm that is functionalized via a flexible linker with a 5 to 50 kDa, preferably 10 to 40 kDa, proteinaceous recognition element R that is specifically reactive with a target analyte, preferably a target protein. In preferred embodiments, R can move in and out of the pore to provoke a blocking current.

In an aspect, the present disclosure provides a sensor system for protein analysis, comprising a fluid-filled compartment separated by a membrane into a first chamber and a second chamber, electrodes capable of applying a potential across the membrane, and at least one biological nanopore that is functionalized with a 5 to 50 kDa, preferably 10 to 40 kDa, proteinaceous recognition element R capable of specifically binding to a target analyte, and wherein R is positioned via a flexible linker atop of the nanopore to allow for moving in and out of the nanopore to provoke transient current blockage events.

In an aspect, the present disclosure provides a nanopore sensor system comprising a cis chamber comprising a first conductive liquid medium in liquid communication with a trans chamber comprising a second conductive liquid medium through a modified nanopore, wherein said modified nanopore is a biological nanopore that is functionalized with a 5 to 50 kDa, preferably 10 to 40 kDa, proteinaceous recognition element R capable of specifically binding to a target analyte, wherein R is tethered atop of the nanopore and is capable of being internalized in the pore and dynamically move in and out of the nanopore lumen to provoke transient current blockage events.

In some embodiments of any one of the preceding embodiments, R is an IgG-based moiety or a non-IgG based moiety, a nanobody, an scFv fragment, a Fab fragment, an affimer, monobody, affibody, Adnectin, DARPin or anticalinmore preferably R is a nanobody. In some embodiments of any one of the preceding embodiments, the biological nanopore is a pore-forming toxin, preferably having a largest internal diameter (e.g., internal lumen diameter) of 5 nm to 20 nm, more preferably selected from the group consisting of cytolysin A (ClyA), pleurotolysin (PlyAB), YaxAB, perforin-2 (PFN2, PDB_ID 6SB3) tripartite alpha-pore forming toxin (AhlB, PDB_ID 6GRJ), C9 (PDB_ID 6DLW) GspD secretin (PDB_ID 5WQ7), Helicobacter pylori OMC (PDB_ID 6X6S), SpoIIIAG (PDB_ID 5WC3), Gasdermin-A3 (PDB_ID 6CB8), or a mutant thereof enabling site specific functionalization with a proteinaceous recognition element. In some embodiments, the biological nanopore is ClyA, preferably a mutant ClyA, more preferably ClyA comprising mutation S110C.

In some embodiments of any one of the preceding embodiments, the flexible linker is an oligonucleotide, preferably duplex DNA, or a chemically modified RNA.

In some embodiments of any one of the preceding embodiments, the nanopore is (reversibly) functionalized with R via a flexible linker L, preferably by nucleic acid hybridization between a first oligonucleotide conjugated to the nanopore and a second oligonucleotide, which is complementary to the first oligonucleotide, conjugated to R.

In an aspect, the present disclosure provides an array comprising a multiplicity of sensor systems according to any one of the preceding embodiments, wherein the array comprises a multiplicity of discrete reservoirs, each of which comprises nanopores modified with different R elements to allow for detection of different analytes.

In an aspect, the present disclosure provides a kit for preparing an array according to the preceding embodiment comprising, nanopores pre-modified with a linker moiety, preferably as part of double-stranded DNA complex composed of the original strand and a complementary protector strand.

In some embodiments of any one of the preceding embodiments, the use of the method, nanopore or sensor system array, or kit can be used in single protein detection, preferable in combination with high throughput analysis. In some embodiments, the sensor system is integrated in a portable device comprising a plurality of sensor systems.

In an aspect, the present disclosure provides a method comprising: (a) providing a nanopore system, wherein the nanopore system comprises (1) a fluid chamber and (2) a membrane comprising a nanopore, wherein the membrane separates the fluid chamber into a first side and a second side, wherein the nanopore is coupled to a recognition element, and (b) contacting the recognition element with an analyte.

In some embodiments, the recognition element is configured to move between an internal region of the nanopore and an external region of the nanopore. In some embodiments, the recognition element is coupled to the nanopore via a linker. In some embodiments, the linker is between about 4 nanometers to about 8 nanometers in length. In some embodiments, the linker comprises an oligonucleotide, a duplex DNA molecule, a chemically modified RNA molecule, or any combination thereof. In some embodiments, the nanopore is coupled to at least a portion of the linker. In some embodiments, the nanopore is coupled to a first oligonucleotide and the linker is coupled to a second oligonucleotide. In some embodiments, the first oligonucleotide and the second oligonucleotide are coupled together via nucleic acid hybridization.

In some embodiments of any one of the preceding embodiments, the nanopore system further comprises a pair of electrodes. In some embodiments, the pair of electrodes are configured to generate an electrical potential across the nanopore. In some embodiments, movement of the recognition element between an internal region of the nanopore and an external region of the nanopore effects a change in a current of the nanopore system. In some embodiments, the method further comprises (c) measuring ionic current passing through an internal region of the nanopore. In some embodiments, the method further comprises (d) detecting presence or absence of the analyte via a change in the ionic current.

In some embodiments of any one of the preceding embodiments, the recognition element is between about 5 kilodaltons to about 50 kilodaltons. In some embodiments of any one of the preceding embodiments, the recognition element couples to the analyte. In some embodiments, the recognition element coupled to the analyte effects movement of the recognition element. In some embodiments, effecting the movement of the recognition element generates a change in (i) a frequency of the movement of the recognition element or (ii) a noise or a magnitude of a current of the nanopore system. In some embodiments, the recognition element cannot move between an internal region of the nanopore and an external region of the nanopore when coupled to the analyte. In some embodiments, the recognition element moves between an internal region of the nanopore and an external region of the nanopore when coupled to the analyte. In some embodiments, a change in (i) a frequency of the movement of the recognition element or (ii) a noise or a magnitude of a current block decreases when the recognition element is coupled to the analyte.

In some embodiments of any one of the preceding embodiments, the nanopore is coupled to another recognition element. In some embodiments, the recognition element and the another recognition element bind to different regions of the analyte. In some embodiments, the recognition element and the another recognition element bind to different analytes.

In some embodiments of any one of the preceding embodiments, the analyte is a protein, a peptide, a small molecule, a protein assembly, a protein/DNA assembly, a protein/RNA assembly, a steroid, a lipid, a lipid membrane, a lipid particle, a bacterium, a viral capsid, a viral particle, a cell, a dendrimer, a polymer, or any combination thereof.

In some embodiments of any one of the preceding embodiments, the analyte is a protein. In some embodiments, the protein is a folded protein, a native protein, a clinically relevant protein, a biomarker, a pathogenic protein, a cell surface protein, or any combination thereof.

In some embodiments of any one of the preceding embodiments, the analyte is from a sample. In some embodiments, the sample is a complex sample. In some embodiments, the complex sample comprises a mixture of proteins. In some embodiments, the sample is a clinical sample. In some embodiments, the clinical sample comprises a bodily fluid. In some embodiments, the bodily fluid comprises whole blood, plasma, serum, urine, feces, saliva, cerebrospinal fluid, breast milk, sputum, or any combination thereof.

In some embodiments of any one of the preceding embodiments, the recognition element is a protein recognition element. In some embodiments, the protein recognition element comprises a nanobody, a Fab fragment, a single-chain variable fragment (scFv), an antibody, a monobody, an affimer, an affibody, an Adnectin, a designed ankyrin repeat protein (DARPin), an anticalin, or any combination thereof.

In some embodiments of any one of the preceding embodiments, the nanopore comprises an oligomeric assembly. In some embodiments, at least one subunit of the oligomeric assembly comprises a subunit of the nanopore coupled to a recognition element. In some embodiments, the recognition element is coupled to the at least one subunit of the nanopore via a linker. In some embodiments, the at least one subunit of the nanopore comprises a monomer of a pore-forming toxin. In some embodiments, the pore-forming toxin comprises cytolysin A (ClyA), pleurotolysin (PlyAB), YaxAB, perforin-2, tripartite alpha-pore forming toxin, secretin, Helicobacter pylori OMC, SpoIIIAG, Gasdermin-A3, or any combination thereof. In some embodiments, the pore-forming toxin comprises one or more mutations. In some embodiments, the pore-forming toxin is ClyA. In some embodiments, the ClyA comprises a S110C mutation.

In some embodiments of any one of the preceding embodiments, an internal region of the nanopore comprises an internal diameter between about 5 nanometers to about 20 nanometers.

In an aspect, the present disclosure provides a method comprising: (a) providing a nanopore system, wherein the nanopore system comprises (1) a fluid chamber and (2) a membrane comprising a nanopore, wherein the membrane separates the fluid chamber into a first side and a second side, wherein the nanopore is coupled to a protein recognition element, wherein the protein recognition element is configured to move between an internal region of the nanopore and an external region of the nanopore, and (b) contacting the protein recognition element with an analyte.

In an aspect, the present disclosure provides a system comprising: (a) a fluid chamber, and (b) a membrane comprising a nanopore, wherein the membrane separates the fluid chamber into (1) a first side and (2) a second side, wherein the nanopore is coupled to a recognition element.

In some embodiments, the recognition element is configured to move between an internal region of the nanopore and an external region of the nanopore.

In some embodiments, the recognition element is coupled to the nanopore via a linker. In some embodiments, the linker is between about 4 nanometers to about 8 nanometers in length. In some embodiments, the linker comprises an oligonucleotide, a duplex DNA molecule, a chemically modified RNA molecule, or any combination thereof. In some embodiments, the nanopore is coupled to at least a portion of the linker. In some embodiments, the nanopore is coupled to a first oligonucleotide and the linker is coupled to a second oligonucleotide. In some embodiments, the first oligonucleotide and the second oligonucleotide are coupled together via nucleic acid hybridization.

In some embodiments of any one of the preceding embodiments, the system further comprises a pair of electrodes. In some embodiments, the pair of electrodes are configured to generate an electrical potential across the nanopore. In some embodiments, movement of the recognition element between an internal region of the nanopore and an external region of the nanopore effects a change in a current of the system.

In some embodiments of any one of the preceding embodiments, the recognition element is between about 5 kilodaltons to about 50 kilodaltons.

In some embodiments of any one of the preceding embodiments, the recognition element is configured to couple to an analyte. In some embodiments, the recognition element coupled to the analyte is configured to effect movement of the recognition element. In some embodiments, the movement of the recognition element generates a change in (i) a frequency of the movement of the recognition element or (ii) a noise or a magnitude of a current of the system. In some embodiments, the recognition element is not configured to move between an internal region of the nanopore and an external region of the nanopore when coupled to the analyte. In some embodiments, the recognition element is configured to move between an internal region of the nanopore and an external region of the nanopore when coupled to the analyte. In some embodiments, a change in (i) a frequency of the movement of the recognition element or (ii) a noise or magnitude of a current block decreases when the recognition element is coupled to the analyte. In some embodiments, the analyte is a protein, a peptide, a small molecule, a protein assembly, a protein/DNA assembly, a protein/RNA assembly, a steroid, a lipid, a lipid membrane, a lipid particle, a bacterium, a viral capsid, a viral particle, a cell, a dendrimer, a polymer, or any combination thereof. In some embodiments, the analyte is a protein. In some embodiments, the protein is a folded protein a native protein, a clinically relevant protein, a biomarker, a pathogenic protein, a cell surface protein, or any combination thereof. In some embodiments, the analyte is from a sample. In some embodiments, the sample is a complex sample. In some embodiments, the complex sample comprises a mixture of proteins. In some embodiments, the sample is a clinical sample. In some embodiments, the clinical sample comprises a bodily fluid. In some embodiments, the bodily fluid comprises whole blood, plasma, serum, urine, feces, saliva, cerebrospinal fluid, breast milk, sputum, or any combination thereof.

In some embodiments of any one of the preceding embodiments, the nanopore is configured to couple to another recognition element. In some embodiments, the recognition element and the another recognition element are configured to bind to different regions of an analyte. In some embodiments, the recognition element and the another recognition element are configured to bind to different analytes.

In some embodiments of any one of the preceding embodiments, the recognition element is a protein recognition element. In some embodiments, the protein recognition element comprises of a nanobody, a Fab fragment, a single-chain variable fragment (scFv), an antibody, a monobody, an affimer, an affibody, an Adnectin, a designed ankyrin repeat protein (DARPin), an anticalin, or any combination thereof.

In some embodiments of any one of the preceding embodiments, the nanopore comprises an oligomeric assembly. In some embodiments, at least one subunit of the oligomeric assembly comprises a subunit of the nanopore coupled to a recognition element. In some embodiments, the recognition element is configured to couple to the at least one subunit of the nanopore via a linker. In some embodiments, the at least one subunit of the nanopore comprises a monomer of a pore-forming toxin. In some embodiments, the pore-forming toxin comprises cytolysin A (ClyA), pleurotolysin (PlyAB), YaxAB, perforin-2, tripartite alpha-pore forming toxin, secretin, Helicobacter pylori OMC, SpoIIIAG, Gasdermin-A3, or any combination thereof. In some embodiments, the pore-forming toxin comprises one or more mutations. In some embodiments, the pore-forming toxin is ClyA. In some embodiments, the ClyA comprises a S110C mutation.

In some embodiments of any one of the preceding embodiments, an internal region of the nanopore comprises an internal diameter between about 5 nanometers to about 20 nanometers.

In an aspect, the present disclosure provides a system comprising: (a) a fluid chamber; and (b) a membrane comprising a nanopore, wherein the membrane separates the fluid chamber into (1) a first side and (2) a second side, wherein the nanopore is coupled to a protein recognition element, wherein the protein recognition element is configured to move between an internal region of the nanopore and an external region of the nanopore.

In an aspect, the present disclosure provides a nanopore comprising a region configured to couple to a recognition element, wherein the recognition element is configured to move between an internal region of the nanopore and an external region of the nanopore.

In some embodiments, the recognition element is coupled to the nanopore via a linker. In some embodiments, the linker is between about 4 nanometers to about 8 nanometers in length. In some embodiments, the linker comprises an oligonucleotide, a duplex DNA complex, a chemically modified RNA complex, or any combination thereof. In some embodiments, the nanopore is coupled to at least a portion of the linker. In some embodiments, the nanopore is coupled to a first oligonucleotide and the linker is coupled to a second oligonucleotide. In some embodiments, the first oligonucleotide and the second oligonucleotide are coupled together via nucleic acid hybridization.

In some embodiments of any one of the preceding embodiments, the recognition element is between about 5 kilodaltons to about 50 kilodaltons.

In some embodiments of any one of the preceding embodiments, the recognition element is configured to couple to an analyte. In some embodiments, the recognition element coupled to the analyte effects movement of the recognition element. In some embodiments, the recognition element is configured to not move between the internal region of the nanopore and the external region of the nanopore when coupled to the analyte. In some embodiments, the recognition element is configured to move between an internal region of the nanopore and an external region of the nanopore when coupled to the analyte. In some embodiments, the analyte is a protein, a peptide, a small molecule, a protein assembly, a protein/DNA assembly, a protein/RNA assembly, a steroid, a lipid, a lipid membrane, a lipid particle, a bacterium, a viral capsid, a viral particle, a cell, a dendrimer, a polymer, or any combination thereof. In some embodiments, the analyte is a protein. In some embodiments, the protein is a folded protein a native protein, a clinically relevant protein, a biomarker, a pathogenic protein, a cell surface protein, or any combination thereof. In some embodiments, the analyte is from a sample. In some embodiments, the sample is a complex sample. In some embodiments, the complex sample comprises a mixture of proteins. In some embodiments, the sample is a clinical sample. In some embodiments, the clinical sample comprises a bodily fluid. In some embodiments, the bodily fluid comprises whole blood, plasma, serum, urine, feces, saliva, cerebrospinal fluid, breast milk, sputum, or any combination thereof.

In some embodiments of any one of the preceding embodiments, the nanopore is configured to couple to another recognition element. In some embodiments, the recognition element and the another recognition element are configured to bind to different regions of an analyte. In some embodiments, the recognition element and the another recognition element are configured to bind to different analytes.

In some embodiments of any one of the preceding embodiments, the recognition element is a protein recognition element. In some embodiments, the protein recognition element comprises a nanobody, a Fab fragment, a single-chain variable fragment (scFv), an antibody, a monobody, an affimer, an affibody, an Adnectin, a designed ankyrin repeat protein (DARPin), an anticalin, or any combination thereof.

In some embodiments of any one of the preceding embodiments, the nanopore comprises an oligomeric assembly. In some embodiments, at least one subunit of the oligomeric assembly comprises a subunit of the nanopore coupled to a recognition element. In some embodiments, the recognition element is configured to couple to the at least one subunit of the nanopore via a linker. In some embodiments, the at least one subunit of the nanopore comprises a monomer of a pore-forming toxin. In some embodiments, the pore-forming toxin comprises cytolysin A (ClyA), pleurotolysin (PlyAB), YaxAB, perforin-2, tripartite alpha-pore forming toxin, secretin, Helicobacter pylori OMC, SpoIIIAG, Gasdermin-A3, or any combination thereof. In some embodiments, the pore-forming toxin comprises one or more mutations. In some embodiments, wherein the pore-forming toxin is ClyA. In some embodiments, the ClyA comprises a S110C mutation.

In an aspect, the present disclosure provides an array comprising a plurality of nanopore system according to any one of the preceding embodiments, wherein the array comprises a multiplicity of discrete reservoirs, wherein one or more of the nanopore systems comprise nanopores modified with different recognition elements to allow for detection of different analytes.

In an aspect, the present disclosure provides a kit for preparing a system of any one of the preceding embodiments, comprising nanopores pre-modified with a linker, wherein the linker is part of a double-stranded DNA complex composed of the original strand and a complementary protector strand.

In an aspect, the present disclosure provides a use of a method, nanopore system, nanopore, array, or kit according to any one of the preceding embodiments for single protein detection, wherein the single protein detection is combined with high throughput analysis. In some embodiments, the sensor system is integrated in a portable device comprising a plurality of sensor systems.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### DETAILED DESCRIPTION

The invention relates to means and methods for analysis of analytes (e.g., target analytes) using nanopore-based sensors. For example, it relates to methods, nanopore systems and devices for the stochastic detection of analytes in complex samples (e.g., detection of a (label-free) protein biomarker in a bodily sample).

Nanopores can stochastically sense single-molecules in real-time and have been used to detect various analytes, such as metal ions^{1 2}, biomolecules^{3 4}, nucleic acids^{5 6 7}, polypeptides^{8 9}. For example, protein sensing^{10 11 12} by this technique holds additional advantages over other existing techniques such as enzyme-linked immunosorbent assays (ELISA) and mass spectrometry, as it can be exploited for protein characterization¹³ and quantification¹⁴, and it can also provide insight into protein unfolding kinetics¹⁵, conformation changes^{16 17 16}, and ligand binding affinity^{18 19}. Besides that, nanopores can readily be integrated into small portable devices²⁰, which makes it very suitable for the application in point-of-care diagnostics.

To date, a variety of nanopore-based strategies have been explored for protein sensing. Straightly, protein detection can be achieved by monitoring the current modulations induced by their direct binding/translocation inside/through the lumen of the pore. The key of this strategy is to choose a pore with appropriate geometry that can accommodate the analyte. In the last decade, nanopores with large lumen areas such as FraC²¹, Cytolysin (ClyA)²², and PlyAB^{23 24} have been exploited for the investigation of folded proteins. For instance, ClyA, with a relatively large (˜6×6×10 nm) cylindrical internal lumen, has shown the ability to capture and characterize different folded proteins²⁵ and distinguish the interaction of peptide or DNA ligands with the protein²². Whereas these biological nanopores have been proven effective, the fixed sizes and the limited types of protein pores available in nature restrict their general application for the sensing of broader variety of folded proteins of variable sizes. In comparison, binder-assisted indirect detection of proteins outside the nanopores has been emerging to be a more generic strategy for folded protein sensing^{14 26 27 28 29 30}.

These approaches variously enable nanopores to detect large proteins that do not fit inside the nanopore, and by exploiting specific binding interactions to proteins enhance the specificity of protein sensing compared to the naked nanopores. The strategies variously involve capturing the protein near the nanopore entrance so as to provoke a change in current from the presence of the analyte, or involve transmitting the binding interactions occurring outside of a nanopore to the interior of the pore, which leads to an altered ionic flow passing through the pore. So far, a variety of binders such as biotins¹⁰, aptamers²⁹, peptides²⁶, protein domains³⁰ have been chemically or genetically functionalized on nanopores, which have been widely used for protein detection or protein-ligand binding studies.

In one example, Thakur and Movileanu established a platform for the investigation of protein-protein interaction³⁰. In this study, a protein domain (RNase barnase, Bn) containing a flexible 12-amino acids peptide adaptor at the N-terminal was fused to a monomeric pore t-FhuA. Upon binding of the cognate ligand protein (Barstar, Bs) to the protein binder, the adaptor was pulled away from the pore opening, which provoked distinguishable unblocking current events³⁰. This nanopore sensor showed the capacity of detecting and quantifying protein analytes in the presence of small amount of serum, however, it had several drawbacks that limited its application in protein sensing. First, constructing nanopores with different protein ligands genetically linked (i.e. fused) to the nanopore is laborious, which makes this approach not optimal for the detection of various distinct proteins. In addition, the preparation of the nanopore requires protein refolding in urea and detergent, which risks making many protein ligands lose their functions.

In another example, Bayley et al., which is incorporated by reference herein in its entirety, demonstrated that an aptamer-modified α-hemolysin (α-HL) nanopore, where a 15mer DNA aptamer (TBA) was hybridized to an oligonucleotide covalently attached to a cysteine near the mouth of the pore, allowed the detection of thrombin²⁹. Remarkably, the anchoring of the DNA adapter on the pore endows it with modularity, thus by changing the aptamer, various analytes can be detected using the same nanopore construct. Nevertheless, it is worth noting that given the diversity of the aptamers' structures and lengths (which range from 15 to 80 bases) for different analytes means that different aptamers cannot be expected to behave in the same manner every time without significant experimentation (e.g. the linker on each aptamer must be carefully adjusted to enable the possibility of detection), thus it is not possible to create a generic system that employs different aptamer binders for different desired analytes.

Soskine et al.¹⁸, which is incorporated by reference herein in its entirety, conjugated aptamers at the top of ClyA nanopores to detect folded proteins by selective external association and pore entry. In this approach, proteins binding to the aptamers above the nanopore are allowed to enter the nanopore, while entry into the nanopore of non-proteins that do not bind to the aptamers is retarded from entry.

Crucially, it can be problematic when employing this platform to biological samples such as blood, as aptamers can be quickly degraded by nucleases.

In some embodiments, the present disclosure provides a novel modular nanopore sensor allowing for the stochastic sensing of (label-free) protein targets, which does not suffer from the drawbacks of known nanopore-based sensors. In some embodiments, the present disclosure provides a generic and versatile system which allows for specific and sensitive detection of proteins and protein-containing pathogens. The present disclosure provides a novel modular nanopore sensor allowing for the stochastic sensing of analytes (e.g., proteins), In some embodiments, the nanopore sensor is a generic and versatile system which allows for specific and sensitive detection of proteins and protein-containing analytes (e.g. viruses, bacteria), including analytes larger than the pore diameter, in complex samples, such as blood or serum.

In some embodiments, the nanopore can be functionalized at the top (or mouth) of a large vestibule nanopore with small (up to about 50 kDa) recognition elements (e.g., proteinaceous recognition elements), such as nanobodies, that can move in and out of the pore to provoke a blocking current. Addition of an analyte (e.g., target analyte) to the solution outside (at the first or cis end) of the pore and the formation of a target-recognition element complex leads to a change in the capture of the recognition elements inside the nanopore, which in turn leads to a change in the ionic current through the open pore. The nanobody (or other small binding molecule) occludes the pore in the resting state, limiting the entry of other proteins. Importantly, in the resting state, the recognition element is mostly lodged inside the nanopore and proteins and other unwanted non-specific background molecules from solution cannot enter the nanopore. Hence, this approach does not suffer from background noise from non-cognate proteins in solution that might otherwise interrupt the signal or block the nanopore.

In some embodiments, the recognition element can be small recognition elements (e.g., proteinaceous recognition elements), this approach is highly specific for detection of a wide range of entities, such as proteins. Secondly, the recognition elements are suitably tethered to the nanopore as replaceable modules, for example by complementary strand hybridization. Thus, nanopores functionalized with different recognition elements (e.g., nanobodies) can be easily acquired and the preparation process is less laborious compared to existing methods. Thirdly, this nuclease-tolerant nanopore design enables the sensing of proteins in biofluids independently of their sizes, in which large proteins will be detected outside of the nanopore, while small proteins will be detected inside the lumen of the pore. In some embodiments, the pore design is exemplified using nanobodies as replaceable modules immobilized on a ClyA dodecamer via DNA duplex formation. By simply changing the modules, four different nanobody-functionalized nanopores were constructed and all of the nanopore constructs showed the capacity for analyte detection. For example, benefiting from the multivalent interaction between SARS-CoV-2 Spike protein and multimerized Ty1 nanobodies, this approach enabled us to detect proteins in the picomolar to low nanomolar range even in the presence of blood. Herewith, the invention provides a novel and generic strategy that allows highly specific and sensitive detection of various proteins regardless of their sizes, shapes and charges in biofluids.

Accordingly, in one embodiment the invention provides a nanopore sensor system comprising a cis chamber comprising a first conductive liquid medium in liquid communication with a trans chamber comprising a second conductive liquid medium through a modified nanopore, wherein said modified nanopore is a biological nanopore that is functionalized with a 5 to 50 kDa, preferably 10 to 40 kDa, proteinaceous recognition element R capable of specifically binding to a target analyte. In one embodiment, the invention provides a nanopore sensor system comprising a first side of the fluid chamber (e.g., cis chamber) comprising a first conductive liquid medium in liquid communication with a second side of the fluid chamber (e.g., trans chamber) comprising a second conductive liquid medium through a modified nanopore, wherein said modified nanopore is a nanopore (e.g., biological nanopore) that is functionalized with a 5 to 50 kDa, for example 10 to 40 kDa, recognition element (e.g., (proteinaceous recognition element) R capable of specifically binding to an analyte (e.g., a target analyte). The recognition element R is suitably tethered atop of the nanopore and, also based on its small dimensions relative to the large lumen dimensions of the pore, is capable of being internalized in the pore. It dynamically moves in and out of the nanopore lumen (vestibule) to provoke transient current blockage events. Binding of R to an analyte modulates its dynamic movement, thereby inducing a change in the frequency and/or magnitude of the current blockage events. The change in the frequency and/or magnitude of current blockage events is indicative of the presence of the analyte in a sample.

In some embodiments, the nanopore of the nanopore sensor system of the present disclosure can be a biological nanopore. Alternatively, in some embodiments, the nanopore of the nanopore sensor system of the present disclosure can be a solid-state nanopore.

In some embodiments, the recognition element can be smaller than the internal diameter (e.g., internal lumen diameter) of the nanopore. In some cases, the recognition element can be between about 0.1% to about 500% smaller than the internal diameter (e.g., internal lumen diameter). In some instances, the recognition element can be between about 0.1% to about 0.5%, between about 0.5% to about 1%, between about 1% to about 5%, between about 5% to about 10%, between about 10% to about 20%, between about 20% to about 30%, between about 30% to about 40%, between about 40% to about 45%, between about 45% to about 50%, between about 50% to about 55%, between about 55% to about 60%, between about 60% to about 65%, between about 65% to about 70%, between about 70% to about 75%, between about 75% to about 80%, between about 80% to about 85%, between about 85% to about 90%, between about 90% to about 95%, between about 95% to about 100%, between about 100% to about 110%, between about 110% to about 120%, between about 120% to about 130%, between about 130% to about 140%, between about 140% to about 150%, between about 150% to about 160%, between about 160% to about 170%, between about 170% to about 180%, between about 180% to about 190%, between about 190% to about 200%, between about 200% to about 210%, between about 210% to about 220%, between about 220% to about 230%, between about 230% to about 240%, between about 240% to about 250%, between about 250% to about 260%, between about 260% to about 270%, between about 270% to about 280%, between about 280% to about 290%, between about 290% to about 300%, between about 300% to about 310%, between about 310% to about 320%, between about 320% to about 330%, between about 330% to about 340%, between about 340% to about 350%, between about 350% to about 360%, between about 360% to about 370%, between about 370% to about 380%, between about 380% to about 390%, between about 390% to about 400%, between about 400% to about 410%, between about 410% to about 420%, between about 420% to about 430%, between about 430% to about 440%, between about 440% to about 450%, between about 450% to about 460%, between about 460% to about 470%, between about 470% to about 480%, between about 480% to about 490%, or between about 490% to about 500% smaller than the internal diameter (e.g., internal lumen diameter) of the nanopore. In some instances, the recognition element can be at least about 0.1%, at least about 0.5%, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more than 500% smaller than the internal diameter (e.g., internal lumen diameter) of the nanopore. In some instances, the recognition element can be at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 1%, at most about 0.5%, at most about 0.1%, or less than 0.1% smaller than the internal diameter (e.g., internal lumen diameter) of the nanopore. In some instances, the recognition element can be about 0.1%, about 0.5%, about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500% smaller than the internal diameter (e.g., internal lumen diameter) of the nanopore.

In some embodiments, the recognition element can move through an external region of the nanopore and an internal region of the nanopore. In some cases, the internal region of the nanopore can be a channel of the nanopore. In some cases, the internal region of the nanopore can be an internal lumen of the nanopore on the first side of the fluid chamber. In some instances, the external region can be any region outside of the channel of the nanopore or the internal lumen of the nanopore. In some cases, the recognition element can move freely between the internal region and the external region of the nanopore. In some instances, moving freely can comprise movement of the recognition element that is not hindered by the linker.

In some embodiments, movement of the recognition element between the internal region of the nanopore and the external region of the nanopore can effect a change in an ionic current of the nanopore. In some embodiments, the movement of the recognition element into the internal region of the nanopore can decrease the ionic current (e.g., magnitude or noise of ionic current) moving through the channel of the nanopore. In some instances, the decrease in the ionic current moving through the channel of the nanopore can be measured.

In some embodiments, the movement of the recognition element into the internal region of the nanopore can block at least a portion of the channel of the nanopore. In some cases, the movement of the recognition element into the internal region of the nanopore can increase the ionic current moving through the channel of the nanopore.

In some embodiments, the movement of the recognition element into the external region of the nanopore can open the channel of the nanopore. In some cases, the movement of recognition element into the external region of the nanopore can increase the ionic current moving through the channel of the nanopore. In some instances, the increase in the ionic current moving through the channel of the nanopore can be measured.

In some embodiments, the nanopore system can exist in two states: (i) the recognition element is in the internal region of the nanopore and (ii) the recognition element is in the external region of the nanopore. In some cases, a change of the nanopore system from (i) to (ii) can be measured. In some cases, a frequency of the nanopore system can be a measurement of the movement between (i) and (ii). In some cases, the nanopore system can measure a change in the frequency of the movement of the recognition element. In some instances, a change in the frequency can indicate presence of the analyte. In some instances, a change in the frequency can indicate absence of the analyte. In some cases, the change in frequency can be used to determine the concentration of the analyte in solution. In some cases, the frequency of the movement of the recognition element in the nanopore system can be between 0.1 kilo Hertz (kHz) to about 1 mega Hertz (MHz). In some cases, the frequency of the nanopore system can be between 0.1 kHz to about 1 kHz, between about 1 kHz to about 100 kHz, or between about 100 kHz to about 1 MHz. In some cases, the frequency of the movement of the recognition element in the nanopore system can be at least about 0.1 kHz, at least about 1 kHz, at least about 5 kHz, at least about 10 kHz, at least about 20 kHz, at least about 30 kHz, at least about 40 kHz, at least about 50 kHz, at least about 60 kHz, at least about 70 kHz, at least about 80 kHz, at least about 90 kHz, at least about 100 kHz, at least about 200 kHz, at least about 300 kHz, at least about 400 kHz, at least about 500 kHz, at least about 600 kHz, at least about 700 kHz, at least about 800 kHz, at least about 900 kHz, at least about 1,000 kHz, at least about 1 MHz, or more than 1 MHz. In some cases, the frequency of the movement of the recognition element in the nanopore system can be at most about 1 MHz, at most about 1,000 kHz, at most about 900 kHz, at most about 800 kHz, at most about 700 kHz, at most about 600 kHz, at most about 500 kHz, at most about 400 kHz, at most about 300 kHz, at most about 200 kHz, at most about 100 kHz, at most about 90 kHz, at most about 80 kHz, at most about 70 kHz, at most about 60 kHz, at most about 50 kHz, at most about 40 kHz, at most about 30 kHz, at most about 20 kHz, at most about 10 kHz, at most about 5 kHz, at most about 1 kHz, at most about 0.1 kHz, or less than 0.1 kHz. In some cases, the frequency of the movement of the recognition element in the nanopore system can be about 0.1 kHz, about 1 kHz, about 5 kHz, about 10 kHz, about 20 kHz, about 30 kHz, about 40 kHz, about 50 kHz, about 60 kHz, about 70 kHz, about 80 kHz, about 90 kHz, about 100 kHz, about 200 kHz, about 300 kHz, about 400 kHz, about 500 kHz, about 600 kHz, about 700 kHz, about 800 kHz, about 900 kHz, about 1,000 kHz, or about 1 MHz.

In some embodiments, the nanopore system can measure a change in a magnitude of the ionic current moving through the nanopore. In some cases, when the recognition element is in the internal region of the nanopore, the ionic current moving through the nanopore can be increased. In some cases, when the recognition element is in the internal region of the nanopore, the ionic current moving through the nanopore can be decreased. In some cases, when the recognition element is in the external region of the nanopore, the ionic current moving through the nanopore can be increased. In some instances, a change in the magnitude of the ionic current can indicate presence of the analyte. In some instances, a change in the magnitude of the ionic current can indicate absence of the analyte. In some cases, the magnitude of the ionic current can be between about 1 picoampere (pA) to about 1,000 pA. In some cases, the magnitude of the ionic current can be between 1 pA to about 10 pA, between about 10 pA to about 100 pA, between about 1 pA to about 100 pA, or between about 100 pA to about 1,000 pA. In some cases, the magnitude of the ionic current can be at least about 1 pA, at least about 5 pA, at least about 10 pA, at least about 20 pA, at least about 30 pA, at least about 40 pA, at least about 50 pA, at least about 60 pA, at least about 70 pA, at least about 80 pA, at least about 90 pA, at least about 100 pA, at least about 200 pA, at least about 300 pA, at least about 400 pA, at least about 500 pA, at least about 600 pA, at least about 700 pA, at least about 800 pA, at least about 900 pA, at least about 1,000 pA, or more than 1,000 pA. In some cases, the magnitude of the ionic current can be at most about 1,000 pA, at most about 900 pA, at most about 800 pA, at most about 700 pA, at most about 600 pA, at most about 500 pA, at most about 400 pA, at most about 300 pA, at most about 200 pA, at most about 100 pA, at most about 90 pA, at most about 80 pA, at most about 70 pA, at most about 60 pA, at most about 50 pA, at most about 40 pA, at most about 30 pA, at most about 20 pA, at most about 10 pA, at most about 5 pA, at most about 1 pA, or less than 1 pA. In some cases, the magnitude of the ionic current can be about 1 pA, about 5 pA, about 10 pA, about 20 pA, about 30 pA, about 40 pA, about 50 pA, about 60 pA, about 70 pA, about 80 pA, about 90 pA, about 100 pA, about 200 pA, about 300 pA, about 400 pA, about 500 pA, about 600 pA, about 700 pA, about 800 pA, about 900 pA, or about 1,000 pA.

In some embodiments, the nanopore system can measure a change in a noise of the ionic current moving through the nanopore. In some cases, the change in the noise of ionic current can refer to a fluctuation (e.g., statistical fluctuation) of the ionic current. In some cases, when the recognition element is in the internal region of the nanopore, the noise of the ionic current can be increased. In some cases, when the recognition element is in the internal region of the nanopore, the noise of the ionic current can be decreased. In some cases, when the recognition element is in the external region of the nanopore, the noise of the ionic current can be increased. In some cases, when the recognition element is in the external region of the nanopore, the noise of the ionic current can be decreased. In some embodiments, the noise of the ionic current can be determined by measuring a change in the frequency of the noise. In some cases, the frequency of the noise can be between 0.1 kilo Hertz (kHz) to about 1 mega Hertz (MHz). In some cases, the frequency of the nanopore system can be between 0.1 kHz to about 1 kHz, between about 1 kHz to about 100 kHz, or between about 100 kHz to about 1 MHz. In some cases, the frequency of the noise can be at least about 0.1 kHz, at least about 1 kHz, at least about 5 kHz, at least about 10 kHz, at least about 20 kHz, at least about 30 kHz, at least about 40 kHz, at least about 50 kHz, at least about 60 kHz, at least about 70 kHz, at least about 80 kHz, at least about 90 kHz, at least about 100 kHz, at least about 200 kHz, at least about 300 kHz, at least about 400 kHz, at least about 500 kHz, at least about 600 kHz, at least about 700 kHz, at least about 800 kHz, at least about 900 kHz, at least about 1,000 kHz, at least about 1 MHz, or more than 1 MHz. In some cases, the frequency of the noise can be at most about 1 MHz, at most about 1,000 kHz, at most about 900 kHz, at most about 800 kHz, at most about 700 kHz, at most about 600 kHz, at most about 500 kHz, at most about 400 kHz, at most about 300 kHz, at most about 200 kHz, at most about 100 kHz, at most about 90 kHz, at most about 80 kHz, at most about 70 kHz, at most about 60 kHz, at most about 50 kHz, at most about 40 kHz, at most about 30 kHz, at most about 20 kHz, at most about 10 kHz, at most about 5 kHz, at most about 1 kHz, at most about 0.1 kHz, or less than 0.1 kHz. In some cases, the frequency of the noise can be about 0.1 kHz, about 1 kHz, about 5 kHz, about 10 kHz, about 20 kHz, about 30 kHz, about 40 kHz, about 50 kHz, about 60 kHz, about 70 kHz, about 80 kHz, about 90 kHz, about 100 kHz, about 200 kHz, about 300 kHz, about 400 kHz, about 500 kHz, about 600 kHz, about 700 kHz, about 800 kHz, about 900 kHz, about 1,000 kHz, or about 1 MHz. In some embodiments, the noise of the ionic current can be determined by measuring a change in the magnitude (e.g., standard deviation) of the noise. In some cases, the magnitude of the noise can be between about 1 picoampere (pA) to about 1,000 pA. In some cases, the magnitude of the noise can be between 1 pA to about 10 pA, between about 10 pA to about 100 pA, between about 1 pA to about 100 pA, or between about 100 pA to about 1,000 pA. In some cases, the magnitude of the noise can be at least about 1 pA, at least about 5 pA, at least about 10 pA, at least about 20 pA, at least about 30 pA, at least about 40 pA, at least about 50 pA, at least about 60 pA, at least about 70 pA, at least about 80 pA, at least about 90 pA, at least about 100 pA, at least about 200 pA, at least about 300 pA, at least about 400 pA, at least about 500 pA, at least about 600 pA, at least about 700 pA, at least about 800 pA, at least about 900 pA, at least about 1,000 pA, or more than 1,000 pA. In some cases, the magnitude of the noise can be at most about 1,000 pA, at most about 900 pA, at most about 800 pA, at most about 700 pA, at most about 600 pA, at most about 500 pA, at most about 400 pA, at most about 300 pA, at most about 200 pA, at most about 100 pA, at most about 90 pA, at most about 80 pA, at most about 70 pA, at most about 60 pA, at most about 50 pA, at most about 40 pA, at most about 30 pA, at most about 20 pA, at most about 10 pA, at most about 5 pA, at most about 1 pA, or less than 1 pA. In some cases, the magnitude of the noise can be about 1 pA, about 5 pA, about 10 pA, about 20 pA, about 30 pA, about 40 pA, about 50 pA, about 60 pA, about 70 pA, about 80 pA, about 90 pA, about 100 pA, about 200 pA, about 300 pA, about 400 pA, about 500 pA, about 600 pA, about 700 pA, about 800 pA, about 900 pA, or about 1,000 pA.

In some embodiments, the recognition element can couple to an analyte. In some cases, the recognition element can couple to a small analyte (e.g., 0.1 nm to 10 nm). In some instances, the recognition element coupled to the small analyte can move between the internal region of the nanopore and the external region of the nanopore. In some instances, the recognition element coupled to the small analyte can move into the internal region of the nanopore. In some cases, the recognition element can couple to a large analyte (e.g., 10 nm or greater). In some instances, the recognition element coupled to the large analyte cannot move between the internal region of the nanopore and the external region of the nanopore. In some instances, the recognition element coupled to the large analyte cannot move into the internal region of the nanopore.

In some embodiments, the recognition element can couple to an analyte. In some cases, the recognition element can specifically couple to the analyte. In some embodiments, the coupling of the recognition element to the analyte can effect the movement of the recognition element. In some cases, the recognition element may not be able to move into the internal region of the nanopore when coupled to the analyte.

In some embodiments, the recognition element coupled to the analyte can be larger than the internal diameter (e.g., internal lumen diameter) of the nanopore. In some cases, the recognition element coupled to the analyte can be between about 0.1% to about 500% larger than the internal diameter (e.g., internal lumen diameter) of the nanopore. In some instances, the recognition element coupled to the analyte can be between about 0.1% to about 0.5%, between about 0.5% to about 1%, between about 1% to about 5%, between about 5% to about 10%, between about 10% to about 20%, between about 20% to about 30%, between about 30% to about 40%, between about 40% to about 45%, between about 45% to about 50%, between about 50% to about 55%, between about 55% to about 60%, between about 60% to about 65%, between about 65% to about 70%, between about 70% to about 75%, between about 75% to about 80%, between about 80% to about 85%, between about 85% to about 90%, between about 90% to about 95%, between about 95% to about 100%, between about 100% to about 110%, between about 110% to about 120%, between about 120% to about 130%, between about 130% to about 140%, between about 140% to about 150%, between about 150% to about 160%, between about 160% to about 170%, between about 170% to about 180%, between about 180% to about 190%, between about 190% to about 200%, between about 200% to about 210%, between about 210% to about 220%, between about 220% to about 230%, between about 230% to about 240%, between about 240% to about 250%, between about 250% to about 260%, between about 260% to about 270%, between about 270% to about 280%, between about 280% to about 290%, between about 290% to about 300%, between about 300% to about 310%, between about 310% to about 320%, between about 320% to about 330%, between about 330% to about 340%, between about 340% to about 350%, between about 350% to about 360%, between about 360% to about 370%, between about 370% to about 380%, between about 380% to about 390%, between about 390% to about 400%, between about 400% to about 410%, between about 410% to about 420%, between about 420% to about 430%, between about 430% to about 440%, between about 440% to about 450%, between about 450% to about 460%, between about 460% to about 470%, between about 470% to about 480%, between about 480% to about 490%, or between about 490% to about 500% larger than the internal diameter (e.g., internal lumen diameter) of the nanopore. In some instances, the recognition element coupled to the analyte can be at least about 0.1%, at least about 0.5%, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more than 500% larger than the internal diameter (e.g., internal lumen diameter) of the nanopore. In some instances, the recognition element coupled to the analyte can be at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 1%, at most about 0.5%, at most about 0.1%, or less than 0.1% larger than the internal diameter (e.g., internal lumen diameter) of the nanopore. In some instances, the recognition element coupled to the analyte can be about 0.1%, about 0.5%, about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500% larger than the internal diameter (e.g., internal lumen diameter) of the nanopore.

In some embodiments, the recognition element coupled to the analyte can be smaller than the internal diameter (e.g., internal lumen diameter) of the nanopore. In some cases, the recognition element coupled to the analyte can be between about 0.1% to about 500% smaller than the internal diameter (e.g., internal lumen diameter) of the nanopore. In some instances, the recognition element coupled to the analyte can be between about 0.1% to about 0.5%, between about 0.5% to about 1%, between about 1% to about 5%, between about 5% to about 10%, between about 10% to about 20%, between about 20% to about 30%, between about 30% to about 40%, between about 40% to about 45%, between about 45% to about 50%, between about 50% to about 55%, between about 55% to about 60%, between about 60% to about 65%, between about 65% to about 70%, between about 70% to about 75%, between about 75% to about 80%, between about 80% to about 85%, between about 85% to about 90%, between about 90% to about 95%, between about 95% to about 100%, between about 100% to about 110%, between about 110% to about 120%, between about 120% to about 130%, between about 130% to about 140%, between about 140% to about 150%, between about 150% to about 160%, between about 160% to about 170%, between about 170% to about 180%, between about 180% to about 190%, between about 190% to about 200%, between about 200% to about 210%, between about 210% to about 220%, between about 220% to about 230%, between about 230% to about 240%, between about 240% to about 250%, between about 250% to about 260%, between about 260% to about 270%, between about 270% to about 280%, between about 280% to about 290%, between about 290% to about 300%, between about 300% to about 310%, between about 310% to about 320%, between about 320% to about 330%, between about 330% to about 340%, between about 340% to about 350%, between about 350% to about 360%, between about 360% to about 370%, between about 370% to about 380%, between about 380% to about 390%, between about 390% to about 400%, between about 400% to about 410%, between about 410% to about 420%, between about 420% to about 430%, between about 430% to about 440%, between about 440% to about 450%, between about 450% to about 460%, between about 460% to about 470%, between about 470% to about 480%, between about 480% to about 490%, or between about 490% to about 500% smaller than the internal diameter (e.g., internal lumen diameter) of the nanopore. In some instances, the recognition element coupled to the analyte can be at least about 0.1%, at least about 0.5%, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more than 500% smaller than the internal diameter (e.g., internal lumen diameter) of the nanopore. In some instances, the recognition element coupled to the analyte can be at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 1%, at most about 0.5%, at most about 0.1%, or less than 0.1% smaller than the internal diameter (e.g., internal lumen diameter) of the nanopore. In some instances, the recognition element coupled to the analyte can be about 0.1%, about 0.5%, about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500% smaller than the internal diameter (e.g., internal lumen diameter) of the nanopore.

In some cases, the movement of the recognition element into the internal region of the nanopore can be decreased when the recognition element is coupled to the analyte. In some instances, the movement of the recognition element into the internal region of the nanopore can be decreased when the recognition element is coupled to the analyte by between about 0.1% to about 500% compared to when the recognition element is not coupled to the analyte. In some cases, the movement of the recognition element into the internal region of the nanopore can be decreased when the recognition element is coupled to the analyte by between about 0.1% to about 0.5%, between about 0.5% to about 1%, between about 1% to about 5%, between about 5% to about 10%, between about 10% to about 20%, between about 20% to about 30%, between about 30% to about 40%, between about 40% to about 45%, between about 45% to about 50%, between about 50% to about 55%, between about 55% to about 60%, between about 60% to about 65%, between about 65% to about 70%, between about 70% to about 75%, between about 75% to about 80%, between about 80% to about 85%, between about 85% to about 90%, between about 90% to about 95%, between about 95% to about 100%, between about 100% to about 110%, between about 110% to about 120%, between about 120% to about 130%, between about 130% to about 140%, between about 140% to about 150%, between about 150% to about 160%, between about 160% to about 170%, between about 170% to about 180%, between about 180% to about 190%, between about 190% to about 200%, between about 200% to about 210%, between about 210% to about 220%, between about 220% to about 230%, between about 230% to about 240%, between about 240% to about 250%, between about 250% to about 260%, between about 260% to about 270%, between about 270% to about 280%, between about 280% to about 290%, between about 290% to about 300%, between about 300% to about 310%, between about 310% to about 320%, between about 320% to about 330%, between about 330% to about 340%, between about 340% to about 350%, between about 350% to about 360%, between about 360% to about 370%, between about 370% to about 380%, between about 380% to about 390%, between about 390% to about 400%, between about 400% to about 410%, between about 410% to about 420%, between about 420% to about 430%, between about 430% to about 440%, between about 440% to about 450%, between about 450% to about 460%, between about 460% to about 470%, between about 470% to about 480%, between about 480% to about 490%, or between about 490% to about 500% compared to when the recognition element is not coupled to the analyte. In some cases, the movement of the recognition element into the internal region of the nanopore can be decreased when the recognition element is coupled to the analyte by at least about 0.1%, at least about 0.5%, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more than 500% compared to when the recognition element is not coupled to the analyte. In some cases, the movement of the recognition element into the internal region of the nanopore can be decreased when the recognition element is coupled to the analyte by at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 1%, at most about 0.5%, at most about 0.1%, or less than 0.1% compared to when the recognition element is not coupled to the analyte. In some cases, the movement of the recognition element into the internal region of the nanopore can be decreased when the recognition element is coupled to the analyte by about 0.1%, about 0.5%, about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500% as compared to when the recognition element is not coupled to the analyte.

In some cases, the movement of the recognition element into the internal region of the nanopore can be increased when the recognition element is coupled to the analyte. In some instances, the movement of the recognition element into the internal region of the nanopore can be increased when the recognition element is coupled to the analyte by between about 0.1% to about 500% compared to when the recognition element is not coupled to the analyte. In some cases, the movement of the recognition element into the internal region of the nanopore can be increased when the recognition element is coupled to the analyte by between about 0.1% to about 0.5%, between about 0.5% to about 1%, between about 1% to about 5%, between about 5% to about 10%, between about 10% to about 20%, between about 20% to about 30%, between about 30% to about 40%, between about 40% to about 45%, between about 45% to about 50%, between about 50% to about 55%, between about 55% to about 60%, between about 60% to about 65%, between about 65% to about 70%, between about 70% to about 75%, between about 75% to about 80%, between about 80% to about 85%, between about 85% to about 90%, between about 90% to about 95%, between about 95% to about 100%, between about 100% to about 110%, between about 110% to about 120%, between about 120% to about 130%, between about 130% to about 140%, between about 140% to about 150%, between about 150% to about 160%, between about 160% to about 170%, between about 170% to about 180%, between about 180% to about 190%, between about 190% to about 200%, between about 200% to about 210%, between about 210% to about 220%, between about 220% to about 230%, between about 230% to about 240%, between about 240% to about 250%, between about 250% to about 260%, between about 260% to about 270%, between about 270% to about 280%, between about 280% to about 290%, between about 290% to about 300%, between about 300% to about 310%, between about 310% to about 320%, between about 320% to about 330%, between about 330% to about 340%, between about 340% to about 350%, between about 350% to about 360%, between about 360% to about 370%, between about 370% to about 380%, between about 380% to about 390%, between about 390% to about 400%, between about 400% to about 410%, between about 410% to about 420%, between about 420% to about 430%, between about 430% to about 440%, between about 440% to about 450%, between about 450% to about 460%, between about 460% to about 470%, between about 470% to about 480%, between about 480% to about 490%, or between about 490% to about 500% compared to when the recognition element is not coupled to the analyte. In some cases, the movement of the recognition element into the internal region of the nanopore can be increased when the recognition element is coupled to the analyte by at least about 0.1%, at least about 0.5%, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more than 500% compared to when the recognition element is not coupled to the analyte. In some cases, the movement of the recognition element into the internal region of the nanopore can be increased when the recognition element is coupled to the analyte by at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 1%, at most about 0.5%, at most about 0.1%, or less than 0.1% compared to when the recognition element is not coupled to the analyte. In some cases, the movement of the recognition element into the internal region of the nanopore can be increased when the recognition element is coupled to the analyte by about 0.1%, about 0.5%, about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500% compared to when the recognition element is not coupled to the analyte.

In some embodiments, when the recognition element is not coupled to the analyte, the recognition element can move between the internal region of the nanopore and the external region of the nanopore. In some cases, when the recognition element is in the internal region of the nanopore, the recognition element can decrease the ionic current (e.g., magnitude or noise of ionic current) passing through the nanopore. In some cases, when the recognition element is in the internal region of the nanopore, the recognition element can increase the ionic current passing through the nanopore. In some instances, the increase and/or decrease in the ionic current can be measured. In some cases, the increase and/or decrease in the ionic current can indicate the absence of the analyte.

In some embodiments, when the recognition element is coupled to the analyte, the internal region of the nanopore can be open. In some cases, when the internal region of the nanopore is open, an ionic current can pass through the nanopore. In some cases, the passage of the ionic current through the nanopore can be measured. In some instances, measuring an ionic current through the nanopore can indicate the presence of the analyte.

In some embodiments, when the recognition element is coupled to the analyte, there can be a decrease in the movement of the recognition element into the internal region of the nanopore. In some cases, when there is a decrease in the movement of the recognition element into the internal region of the nanopore, there can be an increase in the ionic current passing through the nanopore. In some cases, the increase of the ionic current passing through the nanopore can be measured. In some instances, measuring an increase of the ionic current passing through the nanopore can indicate a presence of the analyte. In some instances, measuring an increase of the ionic current passing through the nanopore can indicate an absence of the analyte. In some instances, measuring a decrease of the ionic current passing through the nanopore can indicate a presence of the analyte. In some instances, measuring a decrease of the ionic current passing through the nanopore can indicate an absence of the analyte.

The present disclosure provides a method for detecting the presence of at least one analyte in a sample using a nanopore system comprising a first side of the fluid chamber comprising a first conductive liquid medium in liquid communication with a second side of the fluid chamber comprising a second conductive liquid medium through a modified nanopore, comprising: (a) adding a sample to be analyzed for the presence of an analyte to the first side; (b) optionally applying an electrical potential across the modified nanopore; (c) measuring ionic current passing through the modified nanopore, wherein said modified nanopore is a nanopore (e.g., a biological nanopore) that is functionalized with a 5 to 50 kDa, for example 10 to 40 kDa, recognition elements (e.g., proteinaceous recognition element) R capable of specifically binding to the analyte as defined herein above.

The present disclosure further provides a method for detecting the presence of at least one target analyte in a sample using a nanopore system comprising a cis chamber comprising a first conductive liquid medium in liquid communication with a trans chamber comprising a second conductive liquid medium through a modified nanopore, comprising:
(a) adding a sample to be analyzed for the presence of a target analyte to the cis chamber;
(b) optionally applying an electrical potential across the modified nanopore; and
(c) measuring ionic current passing through the modified nanopore;
   wherein said modified nanopore is a biological nanopore that is functionalized with a 5 to 50 kDa, preferably 10 to 40 kDa, proteinaceous recognition element R capable of specifically binding to the target analyte as defined herein above, preferably wherein R dynamically moves in and out of the nanopore to provoke transient current blockage events, and wherein binding of R to the target analyte modulates its dynamic movement, thereby inducing a change in the frequency and/or magnitude of the current blockage events, and wherein the change in the frequency and/or magnitude of current blockage events indicates the presence of the target analyte in the sample.

WO2016/166232 related to a nanopore-based sensor system comprising a nanopore and a protein adaptor that is internalized in the lumen of the nanopore. The present disclosure provides a nanopore with the recognition element (e.g., a nanobody) is coupled atop of the nanopore and freely moves in and out of the lumen.

In some embodiments, the recognition element (e.g., R) can be a protein recognition element. In some cases, the protein recognition element can be an antibody-based protein molecule. In some instances, the antibody-based protein molecule can be a IgM molecule, a IgG molecule, a IgA molecule, a IgE molecule, a IgD molecule, a IgY molecule, a IgW molecule, a IgT molecule, a IgZ molecule, a nanobody, a scFv, a Fab fragment, or any combination thereof. In some embodiments, the protein recognition element can be a single domain antibody, also known as a nanobody. For example, nanobodies derived from heavy-chain antibodies found in camelids (also known as VHH fragments), or nanobodies derived from the heavy-chain antibodies of Cartilaginous fish (also known as variable new antigen receptor VNAR fragments). Alternatively R can be a Fab fragment, such as an IgG based moiety, for example a single-chain variable fragment (scFv).

Alternatively, in some embodiments, the protein recognition element can be a non-antibody-based protein molecule. In some instances, the non-antibody-based protein molecule can be an affibody, an affilin, an affimer, an affitin, an alphabody, an anticalin, an avimer, a DARPin, a fynomer, a gastrobody, a Kunitz domain peptide, a monobody, a nanoCLAMP, an optimer, a repebody, a pronectin, a centyrin, an obody, or any combination thereof. In some cases, the non-antibody-based protein molecule (e.g., R) can be a non-IgG based moiety, such as those based on affimers, affibodies (based on the Z domain of protein A from Staphylococcus aureus), monobodies and Adnectins (based on the fibronectin type III domain), DARPins (designed ankyrin repeat proteins) or anticalins (based on lipocalins). In some embodiments, the protein recognition element can be an antibody, a nanobody, a scFv, a Fab fragment, an affibody, an affilin, an affimer, an affitin, an alphabody, an anticalin, an avimer, a DARPin, a fynomer, a gastrobody, a Kunitz domain peptide, a monobody, a nanoCLAMP, an optimer, a repebody, a pronectin, a centyrin, an obody, or any combination thereof.

In some embodiments, a nanopore is coupled to an at least about 1 kDa, at least about 2 kDa, at least about 3 kDa, about 4 kDa, at least about 5 kDa, at least about 6 kDa, at least about 7 kDa, at least about 8 kDa, at least about 9 kDa, at least about 10 kDa, at least about 11 kDa, at least about 12 kDa, at least about 13 kDa, at least about 14 kDa, at least about 15 kDa, at least about 16 kDa, at least about 17 kDa, at least about 18 kDa, at least about 19 kDa, at least about 20 kDa, at least about 21 kDa, at least about 22 kDa, at least about 23 kDa, at least about 24 kDa, at least about 25 kDa, at least about 26 kDa, at least about 27 kDa, at least about 28 kDa, at least about 29 kDa, at least about 30 kDa, at least about 31 kDa, at least about 32 kDa, at least about 33 kDa, at least about 34 kDa, at least about 35 kDa, at least about 36 kDa, at least about 37 kDa, at least about 38 kDa, at least about 39 kDa, at least about 40 kDa, at least about 41 kDa, at least about 42 kDa, at least about 43 kDa, at least about 44 kDa, at least about 45 kDa, at least about 46 kDa, at most about 47 kDa, at least about 48 kDa, at least about 49 kDa, at least about 50 kDa, or greater than about 50 kDa recognition element capable of specifically binding to an analyte. In some embodiments, a nanopore is coupled to an at most about 50 kDa, at most about 49 kDa, at most about 48 kDa, at most about 47 kDa, at most about 46 kDa, at most about 45 kDa, at most about 44 kDa, at most about 43 kDa, at most about 42 kDa, at most about 41 kDa, at most about 40 kDa, at most about 39 kDa, at most about 38 kDa, at most about 37 kDa, at most about 36 kDa, at most about 35 kDa, at most about 34 kDa, at most about 33 kDa, at most about 32 kDa, at most about 31 kDa, at most about 30 kDa, at most about 29 kDa, at most about 28 kDa, at most about 27 kDa, at most about 26 kDa, at most about 25 kDa, at most about 24 kDa, at most about 23 kDa, at most about 22 kDa, at most about 21 kDa, at most about 20 kDa, at most about 19 kDa, at most about 18 kDa, at most about 17 kDa, at most about 16 kDa, at most about 15 kDa, at most about 14 kDa, at most about 13 kDa, at most about 12 kDa, at most about 11 kDa, at most about 10 kDa, at most about 9 kDa, at most about 8 kDa, at most about 7 kDa, at most about 6 kDa, at most about 5 kDa, at most about 4 kDa, at most about 3 kDa, at most about 2 kDa, at most about 1 kDa, or less than about 1 kDa recognition element capable of specifically binding to an analyte.

In some embodiments, a nanopore is coupled to an about 5 kDa to about 60 kDa recognition element capable of specifically binding to an analyte. In some embodiments, a nanopore is coupled to an about 5 kDa to about 10 kDa, about 5 kDa to about 15 kDa, about 5 kDa to about 20 kDa, about 5 kDa to about 25 kDa, about 5 kDa to about 30 kDa, about 5 kDa to about 35 kDa, about 5 kDa to about 40 kDa, about 5 kDa to about 45 kDa, about 5 kDa to about 50 kDa, about 5 kDa to about 55 kDa, about 5 kDa to about 60 kDa, about 10 kDa to about 15 kDa, about 10 kDa to about 20 kDa, about 10 kDa to about 25 kDa, about 10 kDa to about 30 kDa, about 10 kDa to about 35 kDa, about 10 kDa to about 40 kDa, about 10 kDa to about 45 kDa, about 10 kDa to about 50 kDa, about 10 kDa to about 55 kDa, about 10 kDa to about 60 kDa, about 15 kDa to about 20 kDa, about 15 kDa to about 25 kDa, about 15 kDa to about 30 kDa, about 15 kDa to about 35 kDa, about 15 kDa to about 40 kDa, about 15 kDa to about 45 kDa, about 15 kDa to about 50 kDa, about 15 kDa to about 55 kDa, about 15 kDa to about 60 kDa, about 20 kDa to about 25 kDa, about 20 kDa to about 30 kDa, about 20 kDa to about 35 kDa, about 20 kDa to about 40 kDa, about 20 kDa to about 45 kDa, about 20 kDa to about 50 kDa, about 20 kDa to about 55 kDa, about 20 kDa to about 60 kDa, about 25 kDa to about 30 kDa, about 25 kDa to about 35 kDa, about 25 kDa to about 40 kDa, about 25 kDa to about 45 kDa, about 25 kDa to about 50 kDa, about 25 kDa to about 55 kDa, about 25 kDa to about 60 kDa, about 30 kDa to about 35 kDa, about 30 kDa to about 40 kDa, about 30 kDa to about 45 kDa, about 30 kDa to about 50 kDa, about 30 kDa to about 55 kDa, about 30 kDa to about 60 kDa, about 35 kDa to about 40 kDa, about 35 kDa to about 45 kDa, about 35 kDa to about 50 kDa, about 35 kDa to about 55 kDa, about 35 kDa to about 60 kDa, about 40 kDa to about 45 kDa, about 40 kDa to about 50 kDa, about 40 kDa to about 55 kDa, about 40 kDa to about 60 kDa, about 45 kDa to about 50 kDa, about 45 kDa to about 55 kDa, about 45 kDa to about 60 kDa, about 50 kDa to about 55 kDa, about 50 kDa to about 60 kDa, or about 55 kDa to about 60 kDa recognition element capable of specifically binding to an analyte.

In some embodiments, a nanopore is coupled to an about 1 kDa, about 2 kDa, about 3 kDa, about 4 kDa, about 5 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa, about 17 kDa, about 18 kDa, about 19 kDa, about 20 kDa, about 21 kDa, about 22 kDa, about 23 kDa, about 24 kDa, about 25 kDa, about 26 kDa, about 27 kDa, about 28 kDa, about 29 kDa, about 30 kDa, about 31 kDa, about 32 kDa, about 33 kDa, about 34 kDa, about 35 kDa, about 36 kDa, about 37 kDa, about 38 kDa, about 39 kDa, about 40 kDa, about 41 kDa, about 42 kDa, about 43 kDa, about 44 kDa, about 45 kDa, about 46 kDa, about 47 kDa, about 48 kDa, about 49 kDa, or about 50 kDa recognition element capable of specifically binding to an analyte.

Alternatively, in some embodiments, the recognition element can be a nucleic acid recognition element. In some cases, the nucleic acid recognition element can be an aptamer. In some cases, the nucleic acid recognition element can be a riboswitch. In some cases, the nucleic acid recognition element can be DNA, RNA, xeno nucleic acid (XNA), locked nucleic acid (LNA), peptide nucleic acid (PNA), bridged nucleic acid (BNA), glycol nucleic acid (GNA), threose nucleic acid (TNA), hexitol nucleic acid (HNA), or any combination thereof. In some instances, the nucleic acid recognition element can be a naturally occurring nucleic acid molecule. In some instances, the nucleic acid recognition element can be a synthetic (e.g., generated in a laboratory) nucleic acid molecule. In some instances, the nucleic acid recognition element can be a recombinant nucleic acid molecule.

In some embodiments, the nucleic acid recognition element can be between about 1 kDA to about 50 kDa in size. In some cases, the nucleic acid recognition element can be between about 1 kDa to about 5 kDa, between about 5 kDa to about 10 kDa, between about 10 kDa to about 15 kDa, between about 15 kDa to about 20 kDa, between about 20 kDa to about 25 kDa, between about 25 kDa to about 30 kDa, between about 30 kDa to about 35 kDa, between about 35 kDa to about 40 kDa, between about 40 kDa to about 45 kDa, or between about 45 kDa to about 50 kDa in size. In some cases, the nucleic acid recognition element can be at least about 1 kDa, at least about 2 kDa, at least about 3 kDa, at least about 4 kDa, at least about 5 kDa, at least about 6 kDa, at least about 7 kDa, at least about 8 kDa, at least about 9 kDa, at least about 10 kDa, at least about 11 kDa, at least about 12 kDa, at least about 13 kDa, at least about 14 kDa, at least about 15 kDa, at least about 16 kDa, at least about 17 kDa, at least about 18 kDa, at least about 19 kDa, at least about 20 kDa, at least about 21 kDa, at least about 22 kDa, at least about 23 kDa, at least about 24 kDa, at least about 25 kDa, at least about 26 kDa, at least about 27 kDa, at least about 28 kDa, at least about 29 kDa, at least about 30 kDa, at least about 31 kDa, at least about 32 kDa, at least about 33 kDa, at least about 34 kDa, at least about 35 kDa, at least about 36 kDa, at least about 37 kDa, at least about 38 kDa, at least about 39 kDa, at least about 40 kDa, at least about 41 kDa, at least about 42 kDa, at least about 43 kDa, at least about 44 kDa, at least about 45 kDa, at least about 46 kDa, at least about 47 kDa, at least about 48 kDa, at least about 49 kDa, at least about 50 kDa, or more in size. In some cases, the nucleic acid recognition element can be at most about 50 kDa, at most about 49 kDa, at most about 48 kDa, at most about 47 kDa, at most about 46 kDa, at most about 45 kDa, at most about 44 kDa, at most about 43 kDa, at most about 42 kDa, at most about 41 kDa, at most about 40 kDa, at most about 39 kDa, at most about 38 kDa, at most about 37 kDa, at most about 36 kDa, at most about 35 kDa, at most about 34 kDa, at most about 33 kDa, at most about 32 kDa, at most about 31 kDa, at most about 30 kDa, at most about 29 kDa, at most about 28 kDa, at most about 27 kDa, at most about 26 kDa, at most about 25 kDa, at most about 24 kDa, at most about 23 kDa, at most about 22 kDa, at most about 21 kDa, at most about 20 kDa, at most about 19 kDa, at most about 18 kDa, at most about 17 kDa, at most about 16 kDa, at most about 15 kDa, at most about 14 kDa, at most about 13 kDa, at most about 12 kDa, at most about 11 kDa, at most about 10 kDa, at most about 9 kDa, at most about 8 kDa, at most about 7 kDa, at most about 6 kDa, at most about 5 kDa, at most about 4 kDa, at most about 3 kDa, at most about 2 kDa, at most about 1 kDa, or less in size. In some cases, the nucleic acid recognition element can be about 1 kDa, about 2 kDa, about 3 kDa, about 4 kDa, about 5 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa, about 17 kDa, about 18 kDa, about 19 kDa, about 20 kDa, about 21 kDa, about 22 kDa, about 23 kDa, about 24 kDa, about 25 kDa, about 26 kDa, about 27 kDa, about 28 kDa, about 29 kDa, about 30 kDa, about 31 kDa, about 32 kDa, about 33 kDa, about 34 kDa, about 35 kDa, about 36 kDa, about 37 kDa, about 38 kDa, about 39 kDa, about 40 kDa, about 41 kDa, about 42 kDa, about 43 kDa, about 44 kDa, about 45 kDa, about 46 kDa, about 47 kDa, about 48 kDa, about 49 kDa, or about 50 kDa in size.

In some embodiments, the nanopore (e.g., biological nanopore) may be functionalized with at least two different recognition elements (e.g., proteinaceous recognition elements) R′ and R″, for example wherein R′ and R″ bind to distinct sites (epitopes) of a given analyte (e.g. target analyte), or wherein R′ and R″ bind to different analytes (e.g. target analytes).

In some embodiments, the analyte (e.g. target analyte) may be a protein, protein assembly, nucleic acid molecule, peptide, small molecule, protein/DNA assembly, protein/RNA assembly, lipid, lipid membrane, carbohydrate, vitamin, lipid particle, oligosaccharide, bacterium, bacterial membrane, a bacterial membrane protein, bacterial nucleic acid, viral nucleic acid, a viral membrane, a viral membrane protein, virus capsid, virus particle, pathogen proteins, pathogen nucleic acid, dendrimer, polymer, or any combination thereof. In one aspect, the analyte (e.g. target analyte) is a protein, for example selected from the group consisting of a folded/native protein, a peptide, a digested folded protein, a clinically relevant protein, biomarker, pathogenic protein, a bacterial protein, a viral protein, a prokaryotic protein, an eukaryotic protein, a parasite protein, an antibody, a contractile protein, an enzyme, a hormonal protein, a structural protein, a storage protein, a transport protein, cell surface protein, or any combination thereof.

The sample can be of any type. It can be an aqueous solution comprising one or more (biological) components. In one aspect, it is a complex sample comprising a mixture of proteins, preferably wherein the sample comprises a clinical sample, more preferably a bodily fluid, such as whole blood, plasma, serum, semen, amniotic fluid, mucus, ascitic fluid, peritoneum, peritoneal fluid, extracellular fluid, interstitial fluid, transcellular fluid, lymphatic fluid, synovial joint fluid, synovial fluid, tears, breast milk, bile, pericardial fluid, gastric acid, pleural fluid, sputum, urine, feces, saliva, cerebrospinal fluid, an aqueous dilution thereof, or any combination thereof.

The nanopore (e.g., biological nanopore) can be a pore-forming toxin, preferably having a largest internal diameter (e.g., internal lumen diameter) of 5 nm to 20 nm. In some cases, the pore is suitably selected from the group consisting of cytolysin A (ClyA), pleurotolysin (PlyAB), YaxAB, perforin-2 (PFN2, PDB_ID 6SB3), tripartite alpha-pore forming toxin (AhlB, PDB_ID 6GRJ), C9 (PDB_ID 6DLW) GspD secretin (PDB_ID 5WQ7), Helicobacter pylori OMC (PDB_ID 6X6S), SpoIIIAG (PDB_ID 5WC3), Gasdermin-A3 (PDB_ID 6CB8), or a mutant thereof enabling site specific functionalization with a recognition element (e.g., proteinaceous recognition element).

In some embodiments, an internal diameter (e.g., internal lumen diameter) can be at least about 5 nm, at least about 6 nm, at least about 7 nm, at least about 8 nm, at least about 9 nm, at least about 10 nm, at least about 11 nm, at least about 12 nm, at least about 13 nm, at least about 14 nm, at least about 15 nm, at least about 16 nm, at least about 17 nm, at least about 18 nm, at least about 19 nm, at least about 20 nm, or greater than about 20 nm. In some embodiments, an internal diameter (e.g., internal lumen diameter) can be at most about 20 nm, at most about 19 nm, at most about 18 nm, at most about 17 nm, at most about 16 nm, at most about 15 nm, at most about 14 nm, at most about 13 nm, at most about 12 nm, at most about 11 nm, at most about 10 nm, at most about 9 nm, at most about 8 nm, at most about 7 nm, at most about 6 nm, at most about 5 nm, or less than about 5 nm.

In some embodiments, an internal diameter (e.g., internal lumen diameter) can be from about 5 nm to about 25 nm. In some embodiments, an internal diameter (e.g., internal lumen diameter) can be from about 5 nm to about 6 nm, about 5 nm to about 7 nm, about 5 nm to about 8 nm, about 5 nm to about 9 nm, about 5 nm to about 10 nm, about 5 nm to about 12 nm, about 5 nm to about 14 nm, about 5 nm to about 16 nm, about 5 nm to about 18 nm, about 5 nm to about 20 nm, about 5 nm to about 25 nm, about 6 nm to about 7 nm, about 6 nm to about 8 nm, about 6 nm to about 9 nm, about 6 nm to about 10 nm, about 6 nm to about 12 nm, about 6 nm to about 14 nm, about 6 nm to about 16 nm, about 6 nm to about 18 nm, about 6 nm to about 20 nm, about 6 nm to about 25 nm, about 7 nm to about 8 nm, about 7 nm to about 9 nm, about 7 nm to about 10 nm, about 7 nm to about 12 nm, about 7 nm to about 14 nm, about 7 nm to about 16 nm, about 7 nm to about 18 nm, about 7 nm to about 20 nm, about 7 nm to about 25 nm, about 8 nm to about 9 nm, about 8 nm to about 10 nm, about 8 nm to about 12 nm, about 8 nm to about 14 nm, about 8 nm to about 16 nm, about 8 nm to about 18 nm, about 8 nm to about 20 nm, about 8 nm to about 25 nm, about 9 nm to about 10 nm, about 9 nm to about 12 nm, about 9 nm to about 14 nm, about 9 nm to about 16 nm, about 9 nm to about 18 nm, about 9 nm to about 20 nm, about 9 nm to about 25 nm, about 10 nm to about 12 nm, about 10 nm to about 14 nm, about 10 nm to about 16 nm, about 10 nm to about 18 nm, about 10 nm to about 20 nm, about 10 nm to about 25 nm, about 12 nm to about 14 nm, about 12 nm to about 16 nm, about 12 nm to about 18 nm, about 12 nm to about 20 nm, about 12 nm to about 25 nm, about 14 nm to about 16 nm, about 14 nm to about 18 nm, about 14 nm to about 20 nm, about 14 nm to about 25 nm, about 16 nm to about 18 nm, about 16 nm to about 20 nm, about 16 nm to about 25 nm, about 18 nm to about 20 nm, about 18 nm to about 25 nm, or about 20 nm to about 25 nm.

In some embodiments, an internal diameter (e.g., internal lumen diameter) can be about 5 nm, about 6 nm, about 7 nm, about 8 nm, about 9 nm, about 10 nm, about 11 nm, about 12 nm, about 13 nm, about 14 nm, about 15 nm, about 16 nm, about 17 nm, about 18 nm, about 19 nm, or about 20 nm.

In some embodiments, the modified nanopore may be an oligomeric assembly comprising or consisting of monomers of the general formula N-L-R, wherein N is a monomer of a pore-forming toxin having a largest internal diameter (e.g., internal lumen diameter) of 5 nm to 20 nm, L is a flexible linker attached to the wide (e.g., cis) entrance of the pore, and R is a recognition elements (e.g., proteinaceous recognition element) capable of specifically binding to the analyte (e.g. target analyte). The flexible linker may have any size as long as it allows for a functional positioning of R relative to the entry/opening of the pore. In one embodiment, L has a length of about 4-8 nm, preferably 5-6 nm. The L may be an oligonucleotide, preferably DNA or chemically modified RNA (e.g. locked nucleic acids or RNA chemically modified at the 2′ position with —F, —OMe to enhance stability. In one embodiment, L contains 8-20 nucleotides, such as 10-18, 12-20, 8-14 or 16-20 nucleotides.

In some embodiments, the recognition element can be coupled to the nanopore in the first side of the nanopore system. In some embodiments, the recognition element can be coupled to the nanopore in the second side of the nanopore system. In some embodiments, the recognition element can be coupled to the nanopore in the first side of the nanopore system and the second side of the nanopore system.

In some embodiments, the analyte can be added to the first side of the nanopore system. In some embodiments, the analyte can be added to the second side of the nanopore system. In some embodiments, the analyte can be added to the first side of the nanopore system and the second side of the nanopore system.

In some embodiments, the recognition element can be coupled to the nanopore in the first side of the nanopore system and the analyte can be added to the first side of the nanopore system. In some embodiments, the recognition element can be coupled to the nanopore in the second side of the nanopore system and the analyte can be added to the second side of the nanopore system. In some embodiments, the recognition element can be coupled to the nanopore in the first side of the nanopore and the second side of the nanopore and the analyte can be added to the first side of the nanopore system and the second side of the nanopore system.

In some embodiments, the recognition element can be coupled to the nanopore. In some cases, the recognition element can be reversibly coupled to the nanopore. In some cases, the recognition element can be irreversible coupled to the nanopore.

In some embodiments, the recognition element can be directly coupled to the nanopore. In some cases, the recognition element can be directly coupled to the nanopore via a covalent bond. In some instances, the covalent bond can be a non-polar covalent bond. In some instances, the covalent bond can be a polar covalent bond. In some cases, the recognition element can be directly coupled to the nanopore via a non-covalent bond. In some instances, the non-covalent bond can be hydrophobic interactions, van der Waals interactions, electrostatic interactions, hydrogen bonds, or any combination thereof.

In some embodiments, the recognition element can be indirectly coupled to the nanopore. In some cases, the recognition element can be indirectly coupled to the nanopore via linker. In some instances, the linker can be a flexible linker. In some cases, the nanopore can be coupled to the linker via a conjugation reaction. In some instances, the conjugation reaction can be a sulfide based conjugative reaction, an ester reaction, a thioester reaction, an amide reaction, a native chemical ligation reaction, or any combination thereof. In some cases, the nanopore can be coupled to the linker via a bioconjugation reaction. In some instances, the bioconjugation reaction can comprise reacting lysine with a N-hydroxysuccinimidyl (NHS) ester, a lysine acylation reaction, reacting lysine with isocyanates, reacting lysine with isothiocyanates, reacting lysine with benzoyl fluorides, reacting cysteine with maleimides, reacting cysteine with iodoacetamides, reacting cysteine with 2-thiopyridine, reacting cysteine with 3-arylpropiolonitrile, an electrophilic aromatic substitution reactions, reacting tyrosine with diazonium salts, reacting tyrosine with 4-phenyl-1,2,4-triazole-3,5-dione (PTAD), a mannich reaction, reacting a N-terminal serine or threonine with NaIO4, reacting N-terminal cysteine with iodoacetamides, reacting N-terminal of analyte with pyridoxal phosphate, staudinger ligation with azides, huisgen cyclization of azides, strain promoted huisgen cyclization of azides, cysteine or Tryptophan RH-catalyzed alkylation, lysine or N-terminal Ir-catalyzed alkylation, tyrosine Pd-catalyzed O-alkylation. cysteine Au-catalyzed alkylation, tryptophan arylation, cysteine arylation, lysine arylation, or any combination thereof.

In some cases, the linker can be an amino acid linker. In some instances, the amino acid linker can comprise any combination of amino acids. In some cases, the canonical amino acids can comprise alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, or any combination thereof. In some cases, the amino acids can be non-natural amino acids. In some cases, the non-natural amino acids can comprise hydroproline, beta-alanine, citrulline, ornithine, norleucine, 3-nitrotyrosine, nitroarginine, pyroglutamic acid, naphtylalanine, Abu, DAB, methionine sulfoxide, methionine sulfone, α-amino-n-butyric acid, norvaline, alloisoleucine, t-leucine, α-amino-n-heptanoic acid, pipecolic acid, allothreonine, homocysteine, homoserine, α,β-diaminopropionic acid, α,γ-diaminobutyric acid, β-alanine, β-amino-n-butyric acid, β-aminoisobutyric acid, β-aminoisobutyric acid, γ-aminobutyric acid, α-aminoisobutyric acid, isovaline, sarcosine, N-ethylglycine, N-propylglycine, N-isopropylglycine, N-methylalanine, N-ethylalanine, N-methyl-β-alanine, N-ethyl-β-alanine, isoserine, α-hydroxy-γ-aminobbutyric acid, or any combinations thereof. In some cases, the linker can comprise any combination of canonical amino acids and non-natural amino acids. In some cases, the amino acid linker can be a combination of glycine and serine amino acids. In some cases, the amino acid linker can be a combination of aspartic acid and serine amino acids. In some instances, the amino acid linker can comprise between about one amino acid to about 10 amino acids. In some instances, the amino acid linker can comprise at least one amino acid, at least about two amino acids, at least about three amino acids, at least about four amino acids, at least about five amino acids, at least about six amino acids, at least about seven amino acids, at least about eight amino acids, at least about nine amino acids, at least about ten amino acids, or more than ten amino acids. In some instances, the amino acid linker can comprise at most about ten amino acids, at most about nine amino acids, at most about eight amino acids, at most about seven amino acids, at most about six amino acids, at most about five amino acids, at most about four amino acids, at most about three amino acids, at most about two amino acids, at most about one amino acid, or less than one amino acid. In some instances, the amino acid linker can comprise about one amino acid, about two amino acids, about three amino acids, about four amino acids, about five amino acids, about six amino acids, about seven amino acids, about eight amino acids, about nine amino acids, or about ten amino acids.

In some cases, the linker can be a polymer linker. In some instances, the polymer linker can be ethylene glycol, polyethylene glycol, or a combination thereof.

In some cases, the linker can be a peptide linker. In some cases, the peptide linker can be a biotin linker. In some cases, the peptide linker can be a streptavidin linker.

In some cases, the linker can be a chemical linker. In some cases, the chemical linker can be a disulfide linker. In some cases, the chemical linker can be a cysteine interaction linker. In some cases, the chemical linker can be a Click chemistry linker. In some cases, the click chemistry linker can involve one or more click reagents. In some cases, the one or more click reagents can include 1,3-dipolar families, epoxides, aziridines, cyclic sulfates, epoxides, aziridines, cyclic sulfates, oxine ethers, hydrazones, aromatic heterocycles, or any combination thereof.

In some cases, the linker can be a nucleic acid linker. In some cases, the nucleic acid linker can be a polynucleic acid linker.

In some embodiments, the linker contains at least about 1 nucleotide, at least about 2 nucleotides, at least about 3 nucleotides, at least about 4 nucleotides, at least about 5 nucleotides, at least about 6 nucleotides, at least about 7 nucleotides, at least about 8 nucleotides, at least about 9 nucleotides, at least about 10 nucleotides, at least about 11 nucleotides, at least about 12 nucleotides, at least about 13 nucleotides, at least about 14 nucleotides, at least about 15 nucleotides, at least about 16 nucleotides, at least about 17 nucleotides, at least about 18 nucleotides, at least about 19 nucleotides, at least about 20 nucleotides, or greater than about 20 nucleotides. In some embodiments, the linker contains at most about 20 nucleotides, at most about 19 nucleotides, at most about 18 nucleotides, at most about 17 nucleotides, at most about 16 nucleotides, at most about 15 nucleotides, at most about 14 nucleotides, at most about 13 nucleotides, at most about 12 nucleotides, at most about 11 nucleotides, at most about 10 nucleotides, at most about 9 nucleotides, at most about 8 nucleotides, at most about 7 nucleotides, at most about 6 nucleotides, at most about 5 nucleotides, at most about 4 nucleotides, at most about 3 nucleotides, at most about 2 nucleotides, at most about 1 nucleotide or less than about 1 nucleotide.

In some embodiments, the linker contains about 1 nucleotide to about 20 nucleotides. In some embodiments, the linker contains about 1 nucleotide to about 2 nucleotides, about 2 nucleotides to about 3 nucleotides, about 3 nucleotides to about 4 nucleotides, about 4 nucleotides to about 5 nucleotides, about 5 nucleotides to about 6 nucleotides, about 6 nucleotides to about 7 nucleotides, about 7 nucleotides to about 8 nucleotides, about 8 nucleotides to about 9 nucleotides, about 8 nucleotides to about 10 nucleotides, about 8 nucleotides to about 12 nucleotides, about 8 nucleotides to about 13 nucleotides, about 8 nucleotides to about 14 nucleotides, about 8 nucleotides to about 15 nucleotides, about 8 nucleotides to about 16 nucleotides, about 8 nucleotides to about 17 nucleotides, about 8 nucleotides to about 18 nucleotides, about 8 nucleotides to about 19 nucleotides, about 8 nucleotides to about 20 nucleotides, about 9 nucleotides to about 10 nucleotides, about 9 nucleotides to about 12 nucleotides, about 9 nucleotides to about 13 nucleotides, about 9 nucleotides to about 14 nucleotides, about 9 nucleotides to about 15 nucleotides, about 9 nucleotides to about 16 nucleotides, about 9 nucleotides to about 17 nucleotides, about 9 nucleotides to about 18 nucleotides, about 9 nucleotides to about 19 nucleotides, about 9 nucleotides to about 20 nucleotides, about 10 nucleotides to about 12 nucleotides, about 10 nucleotides to about 13 nucleotides, about 10 nucleotides to about 14 nucleotides, about 10 nucleotides to about 15 nucleotides, about 10 nucleotides to about 16 nucleotides, about 10 nucleotides to about 17 nucleotides, about 10 nucleotides to about 18 nucleotides, about 10 nucleotides to about 19 nucleotides, about 10 nucleotides to about 20 nucleotides, about 12 nucleotides to about 13 nucleotides, about 12 nucleotides to about 14 nucleotides, about 12 nucleotides to about 15 nucleotides, about 12 nucleotides to about 16 nucleotides, about 12 nucleotides to about 17 nucleotides, about 12 nucleotides to about 18 nucleotides, about 12 nucleotides to about 19 nucleotides, about 12 nucleotides to about 20 nucleotides, about 13 nucleotides to about 14 nucleotides, about 13 nucleotides to about 15 nucleotides, about 13 nucleotides to about 16 nucleotides, about 13 nucleotides to about 17 nucleotides, about 13 nucleotides to about 18 nucleotides, about 13 nucleotides to about 19 nucleotides, about 13 nucleotides to about 20 nucleotides, about 14 nucleotides to about 15 nucleotides, about 14 nucleotides to about 16 nucleotides, about 14 nucleotides to about 17 nucleotides, about 14 nucleotides to about 18 nucleotides, about 14 nucleotides to about 19 nucleotides, about 14 nucleotides to about 20 nucleotides, about 15 nucleotides to about 16 nucleotides, about 15 nucleotides to about 17 nucleotides, about 15 nucleotides to about 18 nucleotides, about 15 nucleotides to about 19 nucleotides, about 15 nucleotides to about 20 nucleotides, about 16 nucleotides to about 17 nucleotides, about 16 nucleotides to about 18 nucleotides, about 16 nucleotides to about 19 nucleotides, about 16 nucleotides to about 20 nucleotides, about 17 nucleotides to about 18 nucleotides, about 17 nucleotides to about 19 nucleotides, about 17 nucleotides to about 20 nucleotides, about 18 nucleotides to about 19 nucleotides, about 18 nucleotides to about 20 nucleotides, or about 19 nucleotides to about 20 nucleotides.

In some embodiments, the linker contains about 1 nucleotide, about 2 nucleotides, about 3 nucleotides, about 4 nucleotides, about 5 nucleotides, about 6 nucleotides, about 7 nucleotides, 8 nucleotides, about 9 nucleotides, about 10 nucleotides, about 11 nucleotides, about 12 nucleotides, about 13 nucleotides, about 14 nucleotides, about 15 nucleotides, about 16 nucleotides, about 17 nucleotides, about 18 nucleotides, about 19 nucleotides, or about 20 nucleotides.

In some embodiments, the nanopore is (reversibly) functionalized with R via linker L, preferably wherein L is formed by nucleic acid hybridization between a first oligonucleotide conjugated to the nanopore and a second oligonucleotide, which is complementary to the first oligonucleotide, conjugated to R.

In one embodiment, the invention provides a nanopore (e.g., modified nanopore, e.g., proteinaceous nanopore) having a minimal pore diameter of 5 nm that is functionalized via a flexible linker with a 5 to 50 kDa, for example 10 to 40 kDa recognition element (e.g., proteinaceous recognition element) R that is specifically reactive with an analyte (e.g. target analyte), for example a protein (e.g., target protein). In some cases, R can move in and out of the pore to provoke a blocking current. In some cases, the recognition element R is tethered atop of the nanopore.

In one aspect, the invention provides a modified proteinaceous nanopore having a minimal pore diameter of 5 nm that is functionalized via a flexible linker with a 5 to 50 kDa, preferably 10 to 40 kDa, proteinaceous recognition element R that is specifically reactive with a target analyte preferably a target protein. In preferred embodiments, R can move in and out of the pore to provoke a blocking current. In preferred embodiments, the recognition element R is tethered atop of the nanopore.

In one embodiment, the invention provides a sensor system for protein analysis, comprising a fluid-filled compartment separated by a membrane into a first chamber and a second chamber, electrodes capable of applying a potential across the membrane, and at least one nanopore (e.g., biological nanopore) that is functionalized with a 5 to 50 kDa, preferably for example 10 to 40 kDa, recognition element (e.g., proteinaceous recognition element) R capable of specifically binding to an analyte, and wherein R is positioned, for example via a flexible linker, atop of the nanopore to allow for moving in and out of the nanopore to provoke transient current blockage events.

In one aspect, the invention provides a sensor system for protein analysis, comprising a fluid-filled compartment separated by a membrane into a first chamber and a second chamber, electrodes capable of applying a potential across the membrane, and at least one biological nanopore that is functionalized with a 5 to 50 kDa, preferably 10 to 40 kDa, recognition element (e.g., proteinaceous recognition element) R capable of specifically binding to a target analyte, and wherein R is positioned preferably via a flexible linked atop of the nanopore to allow for moving in and out of the nanopore to provoke transient current blockage events.

In one embodiment, the invention provides an array comprising a multiplicity of sensor systems according to the invention, and a method and kit for preparing such array. Preferably, the array comprises a multiplicity of discrete reservoirs, each of which comprises nanopores modified with different R elements to allow for detection of different analytes.

In an aspect, the present disclosure provides a kit for preparing an array according to the invention comprising nanopores pre-modified with a linker moiety, preferably as part of double-stranded DNA complex composed of the original strand and a complementary protector strand.

Also provided herein is the use of a method, nanopore or sensor system, array or kit, in single protein detection, preferably in combination with high throughput analysis.

### Definitions

### Recognition Element R

In some embodiments, the recognition element (e.g., proteinaceous recognition element) R is tethered atop of the nanopore and dynamically moves in and out of the nanopore lumen (vestibule) to provoke transient current blockage events. Binding of R to the analyte (e.g., target analyte) modulates this dynamic movement, thereby inducing a change in the frequency and/or magnitude of the current blockage events, wherein the change in the frequency and/or magnitude of current blockage events indicates the presence of the analyte (e.g., target analyte) in the sample. Typically, binding of R to the analyte (e.g., target analyte) increases the time of R staying outside of the pore, thereby decreasing the frequency of the current blockage events.

In some embodiments, to allow for dynamic movement in and out of the vestibule of a nanopore, the R moiety for use in the present invention is much smaller than conventional IgG antibodies, having a molecular weight of approximately 150 kDa, composed of two different kinds of polypeptide chains. The typical dimensions of IgG are approximately 14.5 nm×8.5 nm×4.0 nm, with antigen binding sites separated by 13.7 nm. The molecular weight of R is in the range of 5 to 50 kDa, preferably 10 to 40 kDa, 10 to 35 kDa, 10 to 30 kDa, more preferably 12-15 kDa. Preferred R moieties have dimensions in the single digit nanometer range, such as 1-5×1-5 nm.

In some embodiments, the molecular weight of the recognition element can be at least about 5 kDa, at least about 10 kDa, at least about 15 kDa, at least about 20 kDa, at least about 25 kDa, at least about 30 kDa, at least about 35 kDa, at least about 40 kDa, at least about 45 kDa, at least about 50 kDa, or greater than about 50 kDa. In some embodiments, the molecular weight of recognition element can be at most about 50 kDa, at most about 45 kDa, at most about 40 kDa, at most about 35 kDa, at most about 30 kDa, at most about 25 kDa, at most about 20 kDa, at most about 15 kDa, at most about 10 kDa, at most about 5 kDa, or less than about 5 kDa.

In some embodiments, the molecular weight of recognition element can be from about 5 kDa to about 60 kDa. In some embodiments, the molecular weight of recognition element can be from about 5 kDa to about 10 kDa, about 5 kDa to about 15 kDa, about 5 kDa to about 20 kDa, about 5 kDa to about 25 kDa, about 5 kDa to about 30 kDa, about 5 kDa to about 35 kDa, about 5 kDa to about 40 kDa, about 5 kDa to about 45 kDa, about 5 kDa to about 50 kDa, about 5 kDa to about 55 kDa, about 5 kDa to about 60 kDa, about 10 kDa to about 15 kDa, about 10 kDa to about 20 kDa, about 10 kDa to about 25 kDa, about 10 kDa to about 30 kDa, about 10 kDa to about 35 kDa, about 10 kDa to about 40 kDa, about 10 kDa to about 45 kDa, about 10 kDa to about 50 kDa, about 10 kDa to about 55 kDa, about 10 kDa to about 60 kDa, about 15 kDa to about 20 kDa, about 15 kDa to about 25 kDa, about 15 kDa to about 30 kDa, about 15 kDa to about 35 kDa, about 15 kDa to about 40 kDa, about 15 kDa to about 45 kDa, about 15 kDa to about 50 kDa, about 15 kDa to about 55 kDa, about 15 kDa to about 60 kDa, about 20 kDa to about 25 kDa, about 20 kDa to about 30 kDa, about 20 kDa to about 35 kDa, about 20 kDa to about 40 kDa, about 20 kDa to about 45 kDa, about 20 kDa to about 50 kDa, about 20 kDa to about 55 kDa, about 20 kDa to about 60 kDa, about 25 kDa to about 30 kDa, about 25 kDa to about 35 kDa, about 25 kDa to about 40 kDa, about 25 kDa to about 45 kDa, about 25 kDa to about 50 kDa, about 25 kDa to about 55 kDa, about 25 kDa to about 60 kDa, about 30 kDa to about 35 kDa, about 30 kDa to about 40 kDa, about 30 kDa to about 45 kDa, about 30 kDa to about 50 kDa, about 30 kDa to about 55 kDa, about 30 kDa to about 60 kDa, about 35 kDa to about 40 kDa, about 35 kDa to about 45 kDa, about 35 kDa to about 50 kDa, about 35 kDa to about 55 kDa, about 35 kDa to about 60 kDa, about 40 kDa to about 45 kDa, about 40 kDa to about 50 kDa, about 40 kDa to about 55 kDa, about 40 kDa to about 60 kDa, about 45 kDa to about 50 kDa, about 45 kDa to about 55 kDa, about 45 kDa to about 60 kDa, about 50 kDa to about 55 kDa, about 50 kDa to about 60 kDa, or about 55 kDa to about 60 kDa.

In some embodiments, the molecular weight of recognition element can be about 5 kDa, about 10 kDa, about 15 kDa, about 20 kDa, about 25 kDa, about 30 kDa, about 35 kDa, about 40 kDa, or about 50 kDa.

In some embodiments, the recognition element (e.g., R) can be a single domain antibody, also known as a nanobody. For example, nanobodies derived from heavy-chain antibodies found in camelids (also known as VHH fragments), or nanobodies derived from the heavy-chain antibodies of Cartilaginous fish (also known as variable new antigen receptor VNAR fragments). Alternatively R can be a Fab fragment, such as an IgG based moiety, for example a single-chain variable fragment (scFv). Alternatively, R can be a non-IgG based moiety, such as those based on affimers, affibodies (based on the Z domain of protein A from Staphylococcus aureus), monobodies and Adnectins (based on the fibronectin type III domain), DARPins (designed ankyrin repeat proteins) or anticalins (based on lipocalins).

In one aspect, R is a Fab fragment. In some embodiments, the fragment antigen-binding region (Fab region) is a region on an antibody that binds to antigens. It is composed of one constant and one variable domain of each of the heavy and the light chain. Fab fragment antibodies can be generated by papain digestion of whole IgG antibodies to remove the entire Fc fragment, including the hinge region. These antibodies are monovalent, containing only a single antigen binding site. The molecular weight a Fab fragment is about 50 kDa. The variable domain contains the paratope (the antigen-binding site), comprising a set of complementarity-determining regions, at the amino terminal end of the monomer. Each arm of the Y thus binds an epitope on the antigen.

In another embodiment, R is based on a single-chain variable fragment (scFv), which are fusion proteins of about 30-35 kDa and 2×3 nm that link the variable regions of the heavy (VH) and light chains (VL) of immunoglobulins. See Asaadi et al. (Biomarker Research volume 9, Article number: 87 (2021), which is incorporated by reference herein in its entirety).

In some embodiments, R is a nanobody or a so-called VHH antibody, originally referred to as a heavy chain antibody (HCAb), also known as a single-domain antibody. Nanobodies, which consist of the variable domain of camelid-derived heavy-chain-only antibodies, have emerged as a rapidly growing family of strong protein binders^{31 32 33}. The nomenclature of “nanobody” originally adopted by the Belgian company Ablynx® stemmed from its nanometric size, i.e., 4 nm in length, 2.5 nm in width, and only 12-14 kD in molecular weight. Nanobodies are nuclease-tolerant, which make them more favorable for the indirect protein sensing compared to aptamers. Apart from that, nanobodies can easily be produced as recombinant protein in bacterial expression system and can readily be equipped with customized tags without affecting their function^{35 36 37 38}. Besides, it has been reported that nanobody multimerization can improve its binding affinity³⁹ and enhance detection sensitivity⁴⁰.

In some embodiments, R is a non-IgG based moiety, such as an affimer. Affimer® molecules are small proteins that bind to analytes (e.g., target analytes) with affinity in the nanomolar range. At 12-14 kDa, Affimer reagents are small non-antibody binding proteins, ˜10 times smaller than IgG antibodies and they are less than 4 nm in length. These engineered non-antibody binding proteins are designed to mimic the molecular recognition characteristics of monoclonal antibodies in different applications.

In a preferred aspect R is a nanobody or a so-called VHH antibody, originally referred to as a heavy chain antibody (HCAb), also known as a single-domain antibody, as described herein above. In some embodiments, R is a non IgG based moiety, such as an affimer. Affimer® molecules are small proteins that bind to target proteins with affinity in the nanomolar range, as described herein above.

See also Bedford et al. (Biophysical Reviews volume 9, pg. 299-308; 2017, which is incorporated by reference herein in its entirety) providing examples of smaller size immunoglobulin G (IgG) and non-IgG-based binding reagents that are suitably used for nanopore functionalization.

In some embodiments, one nanopore may be conjugated to the same type of R moiety, e.g. all being nanobodies, scFv or affimers, or to mixed type of R moieties, e.g. a combination of two or more types of those listed herein above, such as scFv and affimers or scFv and nanobodies.

In some embodiments, the nanopore can be coupled to one or more recognition elements. In some embodiments, the nanopore can be coupled to at least about 1 recognition element, at least about 2 recognition elements, at least about 3 recognition elements, at least about 4 recognition elements, at least about 5 recognition elements, at least about 10 recognition elements, at least about 12 recognition elements, at least about 15 recognition elements, at least about 18 recognition elements, at least about 20 recognition elements, at least about 25 recognition elements, at least about 30 recognition elements, at least about 35 recognition elements, at least about 40 recognition elements, at least about 45 recognition elements, at least about 50 recognition elements, or greater than about 50 recognition elements. In some embodiments, the nanopore can be coupled to at most about 50 recognition elements, at most about 45 recognition elements, at most about 40 recognition elements, at most about 35 recognition elements, at most about 30 recognition elements, at most about 25 recognition elements, at most about 20 recognition elements, at most about 18 recognition elements, at most about 15 recognition elements, at most about 12 recognition elements, at most about 10 recognition elements, at most about 5 recognition elements, at most about 4 recognition elements, at most about 3 recognition elements, at most about 2 recognition elements, at most about 1 recognition element, or less than 1 recognition element.

In some embodiments, the nanopore can be coupled to from about 1 recognition element to about 50 recognition elements. In some embodiments, the nanopore can be coupled to from about 1 recognition element to about 2 recognition elements, about 1 recognition element to about 3 recognition elements, about 1 recognition element to about 4 recognition elements, about 1 recognition element to about 5 recognition elements, about 1 recognition element to about 10 recognition elements, about 1 recognition element to about 15 recognition elements, about 1 recognition element to about 20 recognition elements, about 1 recognition element to about 25 recognition elements, about 1 recognition element to about 30 recognition elements, about 1 recognition element to about 40 recognition elements, about 1 recognition element to about 50 recognition elements, about 2 recognition elements to about 3 recognition elements, about 2 recognition elements to about 4 recognition elements, about 2 recognition elements to about 5 recognition elements, about 2 recognition elements to about 10 recognition elements, about 2 recognition elements to about 15 recognition elements, about 2 recognition elements to about 20 recognition elements, about 2 recognition elements to about 25 recognition elements, about 2 recognition elements to about 30 recognition elements, about 2 recognition elements to about 40 recognition elements, about 2 recognition elements to about 50 recognition elements, about 3 recognition elements to about 4 recognition elements, about 3 recognition elements to about 5 recognition elements, about 3 recognition elements to about 10 recognition elements, about 3 recognition elements to about 15 recognition elements, about 3 recognition elements to about 20 recognition elements, about 3 recognition elements to about 25 recognition elements, about 3 recognition elements to about 30 recognition elements, about 3 recognition elements to about 40 recognition elements, about 3 recognition elements to about 50 recognition elements, about 4 recognition elements to about 5 recognition elements, about 4 recognition elements to about 10 recognition elements, about 4 recognition elements to about 15 recognition elements, about 4 recognition elements to about 20 recognition elements, about 4 recognition elements to about 25 recognition elements, about 4 recognition elements to about 30 recognition elements, about 4 recognition elements to about 40 recognition elements, about 4 recognition elements to about 50 recognition elements, about 5 recognition elements to about 10 recognition elements, about 5 recognition elements to about 15 recognition elements, about 5 recognition elements to about 20 recognition elements, about 5 recognition elements to about 25 recognition elements, about 5 recognition elements to about 30 recognition elements, about 5 recognition elements to about 40 recognition elements, about 5 recognition elements to about 50 recognition elements, about 10 recognition elements to about 15 recognition elements, about 10 recognition elements to about 20 recognition elements, about 10 recognition elements to about 25 recognition elements, about 10 recognition elements to about 30 recognition elements, about 10 recognition elements to about 40 recognition elements, about 10 recognition elements to about 50 recognition elements, about 15 recognition elements to about 20 recognition elements, about 15 recognition elements to about 25 recognition elements, about 15 recognition elements to about 30 recognition elements, about 15 recognition elements to about 40 recognition elements, about 15 recognition elements to about 50 recognition elements, about 20 recognition elements to about 25 recognition elements, about 20 recognition elements to about 30 recognition elements, about 20 recognition elements to about 40 recognition elements, about 20 recognition elements to about 50 recognition elements, about 25 recognition elements to about 30 recognition elements, about 25 recognition elements to about 40 recognition elements, about 25 recognition elements to about 50 recognition elements, about 30 recognition elements to about 40 recognition elements, about 30 recognition elements to about 50 recognition elements, or about 40 recognition elements to about 50 recognition elements.

In some embodiments, the nanopore can be coupled to about 1 recognition element, about 2 recognition elements, about 3 recognition elements, about 4 recognition elements, about 5 recognition elements, about 10 recognition elements, about 12 recognition elements, about 15 recognition elements, about 18 recognition elements, about 20 recognition elements, about 25 recognition elements, about 30 recognition elements, about 35 recognition elements, about 40 recognition elements, about 45 recognition elements, or about 50 recognition elements.

In some embodiments, the one or more recognition elements can couple to the same region of the analyte. In some embodiments, the one or more recognition elements can couple to different regions of the analyte. In some embodiments, the one or more recognition element can couple to different analytes.

In some embodiments, R is coupled or positioned via a flexible tether atop of the nanopore (e.g., at the cis side atop of the nanopore) allowing for contacting the analyte that is added to the first side (e.g., cis chamber). The site of linker coupling to R is chosen on a surface, loop or termini of the protein such that it leaves the binding domain motifs of R free and sterically unhindered. Common conjugation sites (e.g. for binding R to beads or surfaces) are well known for many suitable R binders. The site of nanopore modification with R is chosen such that it allows R to dynamically move in and out of the interior of the nanopore, or at least to provoke transient current blockage events in the absence of analyte (e.g. target analyte), and wherein binding of analyte (e.g. target) to R modulates its dynamic movement, thereby inducing a change in the frequency and/or magnitude of the current blockage events.

In some embodiments, binding of R to the analyte (e.g. target analyte) increases the time of R staying outside of the pore, for example through steric or electrostatic effects that reduce the ability of the R-analyte complex to enter the nanopore cavity, thereby decreasing the frequency of the current blockage events. Alternatively, binding of R to the analyte (e.g. target analyte) reduces the time of R staying outside of the pore, thereby increasing the frequency of the current blockage events. For example, binding to a highly charged analyte may aid in internalization through changes to electrophoretic forces acting on the R-analyte complex.

In other embodiments, binding of R to the analyte (e.g. target analyte) alters the ionic current flow through the nanopore when the R-analyte complex is inside the nanopore. For example, in embodiments where the R-analyte complex is able to enter the nanopore cavity, the presence of the analyte either increases or decreases the ionic current flowing through nanopore relative to the unbound R current level as a result of a change in excluded volume or electrostatics. In some embodiments the R-analyte complex exhibits multiple current levels as a result of the complex being located at different positions within the nanopore. The changes in current level can be used to detect the presence of the analyte. The changes and absolute values of the current levels associated with R-analyte complex inside the nanopore can also be used to determine other properties of the analyte, such as for example the presence and type of one or more post-translational modifications (e.g. phosphorylations, glycosylations, etc.). For example, R may be designed to universally bind to a specific analyte (e.g., specific target protein analyte) that is present in multiple post-translationally or otherwise modified forms in a mixture, for example binding to unmodified epitope region of the protein, so that the modified regions of the protein analyte facing into the nanopore alter the ionic current in a distinctive manner.

In some embodiments, the nanopore (e.g., biological nanopore) may be functionalized with one type of R allowing for sensing one analyte (e.g., target analyte), or it may be functionalized with at least two different recognition elements (e.g., proteinaceous recognition elements) R′ and R″. In one embodiment, the nanopore is functionalized with at least R′ and R″, each of which binds specifically to a different analyte (e.g., target analyte), thus enabling a single nanopore to detect multiple different analytes (e.g., target analytes). In preferred embodiments, the nanopore is functionalized with at least R′ and R″, each binding specifically to distinct sites (epitopes) of the same analyte (e.g., target analyte). In this way the binding strength and duration of the analyte bound state can be increased, as well as increasing the specificity for binding the given analyte (e.g., target analyte) over other background analytes.

In some embodiments, R is preferably positioned via a flexible tether at the first side (or at the cis side) atop of the nanopore. This flexible tether allows R to move in and out of the pore as described herein.

In one aspect, recognition element R is directly coupled to the nanopore. In one aspect, flexibility, e.g., allowing rotation or bending of R so that it can move in and out of the pore as described herein, may be achieved by the bond via which R is attached to the nanopore. In one aspect, such flexibility may be achieved by flexibility within R or within the nanopore. In some examples, the nanopore may be conjugated to a flexible region of R such as flexible N- or C-termini, or flexible loops on the outer surface of R. Alternatively or in combination, R may be coupled to flexible regions of the nanopore, such as flexible N- or C-termini, or flexible loops on the outer surface of the nanopore.

In one aspect, the modified nanopore is an oligomeric assembly comprising or consisting of monomers of the general formula N-L-R, wherein N is a monomer of a pore-forming toxin having a largest internal diameter (e.g., internal lumen diameter) of 5 nm to 20 nm, L is a flexible linker attached to the wide entrance (e.g., a wide cis entrance) of the pore, and R is a recognition element (e.g., proteinaceous recognition element) capable of specifically binding to the analyte (e.g., target analyte).

In some embodiments, the nanopore is a monomeric protein. In some cases, the nanopore can be formed of a single beta-barrel similar to outer membrane porin structures. In some embodiments, the nanopore can be a monomer formed by genetic fusion or chemical conjugation of multiple monomeric protein units.

In some embodiments, the nanopore can comprise an oligometric assembly. In some cases, at least one subunit of the oligometric assembly comprises a subunit of the nanopore coupled to a recognition element. In some cases, the at least one subunit can be directly coupled to the recognition element. In some cases, the at least one subunit can be indirectly coupled to the recognition element. In some cases, the at least one subunit can be coupled to the at least one subunit of the nanopore via a linker. In some instances, the at least one subunit of the nanopore comprises a monomer of a pore-forming toxin. In some instances, the pore-forming toxin can be cytolysin A (ClyA), pleurotolysin (PlyAB), YaxAB, perforin-2, tripartite alpha-pore forming toxin, secretin, Helicobacter pylori OMC, SpoIIIAG, Gasdermin-A3, or any combination thereof. In some cases, the pore-forming toxin can comprise one or more mutations. In some cases, the pore-forming toxin is ClyA. In some cases, the ClyA pore-forming toxin can have a S110C mutation.

In some embodiments, the nanopore has an internal diameter (e.g., internal lumen diameter) can be at least about 5 nm, at least about 6 nm, at least about 7 nm, at least about 8 nm, at least about 9 nm, at least about 10 nm, at least about 11 nm, at least about 12 nm, at least about 13 nm, at least about 14 nm, at least about 15 nm, at least about 16 nm, at least about 17 nm, at least about 18 nm, at least about 19 nm, at least about 20 nm, or greater than about 20 nm. In some embodiments, the nanopore has an internal diameter (e.g., internal lumen diameter) can be at most about 20 nm, at most about 19 nm, at most about 18 nm, at most about 17 nm, at most about 16 nm, at most about 15 nm, at most about 14 nm, at most about 13 nm, at most about 12 nm, at most about 11 nm, at most about 10 nm, at most about 9 nm, at most about 8 nm, at most about 7 nm, at most about 6 nm, at most about 5 nm, or less than about 5 nm.

In some embodiments, the nanopore has an internal diameter (e.g., internal lumen diameter) can be from about 5 nm to about 25 nm. In some embodiments, the nanopore has an internal diameter (e.g., internal lumen diameter) can be from about 5 nm to about 6 nm, about 5 nm to about 7 nm, about 5 nm to about 8 nm, about 5 nm to about 9 nm, about 5 nm to about 10 nm, about 5 nm to about 12 nm, about 5 nm to about 14 nm, about 5 nm to about 16 nm, about 5 nm to about 18 nm, about 5 nm to about 20 nm, about 5 nm to about 25 nm, about 6 nm to about 7 nm, about 6 nm to about 8 nm, about 6 nm to about 9 nm, about 6 nm to about 10 nm, about 6 nm to about 12 nm, about 6 nm to about 14 nm, about 6 nm to about 16 nm, about 6 nm to about 18 nm, about 6 nm to about 20 nm, about 6 nm to about 25 nm, about 7 nm to about 8 nm, about 7 nm to about 9 nm, about 7 nm to about 10 nm, about 7 nm to about 12 nm, about 7 nm to about 14 nm, about 7 nm to about 16 nm, about 7 nm to about 18 nm, about 7 nm to about 20 nm, about 7 nm to about 25 nm, about 8 nm to about 9 nm, about 8 nm to about 10 nm, about 8 nm to about 12 nm, about 8 nm to about 14 nm, about 8 nm to about 16 nm, about 8 nm to about 18 nm, about 8 nm to about 20 nm, about 8 nm to about 25 nm, about 9 nm to about 10 nm, about 9 nm to about 12 nm, about 9 nm to about 14 nm, about 9 nm to about 16 nm, about 9 nm to about 18 nm, about 9 nm to about 20 nm, about 9 nm to about 25 nm, about 10 nm to about 12 nm, about 10 nm to about 14 nm, about 10 nm to about 16 nm, about 10 nm to about 18 nm, about 10 nm to about 20 nm, about 10 nm to about 25 nm, about 12 nm to about 14 nm, about 12 nm to about 16 nm, about 12 nm to about 18 nm, about 12 nm to about 20 nm, about 12 nm to about 25 nm, about 14 nm to about 16 nm, about 14 nm to about 18 nm, about 14 nm to about 20 nm, about 14 nm to about 25 nm, about 16 nm to about 18 nm, about 16 nm to about 20 nm, about 16 nm to about 25 nm, about 18 nm to about 20 nm, about 18 nm to about 25 nm, or about 20 nm to about 25 nm.

In some embodiments, the nanopore has an internal diameter (e.g., internal lumen diameter) can be about 5 nm, about 6 nm, about 7 nm, about 8 nm, about 9 nm, about 10 nm, about 11 nm, about 12 nm, about 13 nm, about 14 nm, about 15 nm, about 16 nm, about 17 nm, about 18 nm, about 19 nm, or about 20 nm.

In some embodiments, the linker size is variable and may depends among others on the site of R attachment, pore dimensions and/or pore geometry. A person of skill in the art will be able to readily select suitable linker lengths and linker geometries. In some embodiments, suitable linker lengths and linker geometries can be linkers that, in combination with the location of the linker attachment point on the nanopore, provide a suitable distance of R from the pore entrance.

In some embodiments, the flexible linker may have any size as long as it allows for a functional positioning of R relative to the entry/opening of the pore. In one embodiment, L has a length of about 2-8 nm, preferably 4-6 nm. It is to be understood that this length of L is the shortest distance between the nanopore attachment point and R attachment point; the rest of the linker moiety can be almost any length.

In some embodiments, the linker has a length of at least about 0.5 nm, at least about 1 nm, at least about 1.5 nm, at least about 2.0 nm, at least about 2.5 nm, at least about 3 nm, at least about 3.5 nm, at least about 4 nm, at least about 4.5 nm, at least about 5 nm, at least about 5.5 nm, at least about 6 nm, at least about 6.5 nm, at least about 7 nm, at least about 7.5 nm, at least about 8 nm, or greater than about 8 nm. In some embodiments, the linker has a length of at most about 8 nm, at most about 7.5 nm, at most about 7 nm, at most about 6.5 nm, at most about 6 nm, at most about 5.5 nm, at most about 5 nm, at most about 4.5 nm, at most about 4 nm, at most about 3.5 nm, at most about 3 nm, at most about 2.5 nm, at most about 2 nm, at most about 1.5 nm, at most about 1.0 nm, at most about 0.5 nm, or less than about 0.5 nm.

In some embodiments, the linker has a length from about 0.5 nm to about 8 nm. In some embodiments, the linker has a length from about 0.5 nm to about 1.0 nm, from about 1.0 nm to about 1.5 nm, from about 1.5 nm to about 2.0 nm, from about 2 nm to about 2.5 nm, about 2 nm to about 3 nm, about 2 nm to about 3.5 nm, about 2 nm to about 4 nm, about 2 nm to about 4.5 nm, about 2 nm to about 5 nm, about 2 nm to about 5.5 nm, about 2 nm to about 6 nm, about 2 nm to about 7 nm, about 2 nm to about 8 nm, about 2.5 nm to about 3 nm, about 2.5 nm to about 3.5 nm, about 2.5 nm to about 4 nm, about 2.5 nm to about 4.5 nm, about 2.5 nm to about 5 nm, about 2.5 nm to about 5.5 nm, about 2.5 nm to about 6 nm, about 2.5 nm to about 7 nm, about 2.5 nm to about 8 nm, about 3 nm to about 3.5 nm, about 3 nm to about 4 nm, about 3 nm to about 4.5 nm, about 3 nm to about 5 nm, about 3 nm to about 5.5 nm, about 3 nm to about 6 nm, about 3 nm to about 7 nm, about 3 nm to about 8 nm, about 3.5 nm to about 4 nm, about 3.5 nm to about 4.5 nm, about 3.5 nm to about 5 nm, about 3.5 nm to about 5.5 nm, about 3.5 nm to about 6 nm, about 3.5 nm to about 7 nm, about 3.5 nm to about 8 nm, about 4 nm to about 4.5 nm, about 4 nm to about 5 nm, about 4 nm to about 5.5 nm, about 4 nm to about 6 nm, about 4 nm to about 7 nm, about 4 nm to about 8 nm, about 4.5 nm to about 5 nm, about 4.5 nm to about 5.5 nm, about 4.5 nm to about 6 nm, about 4.5 nm to about 7 nm, about 4.5 nm to about 8 nm, about 5 nm to about 5.5 nm, about 5 nm to about 6 nm, about 5 nm to about 7 nm, about 5 nm to about 8 nm, about 5.5 nm to about 6 nm, about 5.5 nm to about 7 nm, about 5.5 nm to about 8 nm, about 6 nm to about 7 nm, about 6 nm to about 8 nm, or about 7 nm to about 8 nm.

In some embodiments, the linker has a length of about 0.5 nm, about 1.0 nm, about 1.5 nm, about 2 nm, about 2.5 nm, about 3 nm, about 3.5 nm, about 4 nm, about 4.5 nm, about 5 nm, about 5.5 nm, about 6 nm, about 6.5 nm, about 7 nm, about 7.5 nm, or about 8 nm.

In some embodiments, linkers can be composed of many well known types, including polymers such as PEG, DNA,RNA,LNA,PNA, or any combination thereof. In some cases, the one or more polymeric molecules can comprise polyethylene glycol, ethylene, polystyrene, vinyl chloride, polyethylene, polypropylene, polycarbonates, polytetrafluoroethylene, polyamide, silicone based polymers, PMOXA polymers, polyglycans, polyacrylamide polymers, polyacrylic acid polymers, polyamines, polyethyleneimines, quaternary ammonium polymers, polyvinyl alcohol polymers, pluronic polymers, ethylene oxide polymers, propylene oxide polymers, polyvinylpyrrolidone polymers, carboxypolymethylene polymers, or any combination thereof. Conjugation can employ any suitable well known chemical attachment methods, such as those involving reactions with cysteines (e.g. maleimide coupling), lysines, Click chemistry etc. Conjugation chemistries are preferably located at the ends of the linkers, but can be located part way along the molecule as required to locate the R relative the N. The linker can be composed of a single unit (e.g. a single polymer chain that directly couples N to R) or multiple units (eg. Hybridized oligonucleotides where R and N each are bound to one of the duplex strands). R is suitably coupled directly to N as long as the N attachment point is provided with sufficient length and flexibility, so as to allow R to move in and out of the nanopore. This can be achieved by example by attaching R to a flexible loop on N that exists, or is introduced into the N sequence at the appropriate position. Alternatively, R can be engineered with an additional sequence (eg. An internal loop or an extension of the N- or C-termini if there attached) to create a flexible linker, then attached directly to a surface residue on N.

In one embodiment L is an oligonucleotide, preferably duplex made of complementary strands of DNA, or chemically modified RNA (e.g. locked nucleic acids or RNA chemically modified at the 2′ position with —F, —OMe to enhance stability It may comprise at stretch of at least 8, least 10, preferably at least 14, more preferably at least 18 nucleotides.

In some embodiments, the nanopore is (reversibly) functionalized with R via linker L, preferably wherein L is an oligonucleotide duplex formed by nucleic acid hybridization between a first oligonucleotide conjugated to the nanopore and a second oligonucleotide, which is complementary to the first oligonucleotide, conjugated to R.

In some embodiments, different orientations are possible to locate R favorably relative to the L attachment point on the nanopore and to the nanopore entrance (see FIGS. 14A-14C). For example, N and R might be coupled to the same end of an oligonucleotide duplex linker L. Alternatively, N and R might be coupled to the mid points or far ends of an oligonucleotide duplex linker L.

in some embodiments, duplex formation and exchange of the components attached to the nanopore is suitably achieved by toehold mediated strand displacement (TMSD) reaction, involving a process in which an invader strand displaces the incumbent strand from the gate strand through initiation at the exposed toehold domain. For example, TMSD can be used to exchange the strand duplexed to the nanopore. For example, for a nanopore comprising N-L1 (where L1 is one strand of a duplex linker), initially duplexed to L2-R1, TMSD can be used to exchange the bound entity to L3-R2 to change the coupled binder R, and thus the analyte (e.g., target) that the nanopore is able to detect.

In another embodiment the linker L on the nanopore is first protected, so that it is only activated when desired. For example, the nanopore is initially duplexed to a blank protecting strand, which is removed on a channel-by-channel basis on a chip array containing multiple nanopores. For example, the blank protecting oligonucleotide strand can be removed from a desired nanopore on a channel-by-channel basis by applying voltage to the selected channel containing a nanopore so as to capture and electrophoretically strip the protecting strand from the nanopore (FIGS. 15A-15D).

### Nanopore

In some embodiments, the nanopore suitably has a largest internal diameter (e.g., internal lumen diameter) of 5 nm to 20 nm, such as 5 to 10 nm. The pore may have a wide entrance (e.g., a wide cis entrance) and a narrow exit (e.g., a narrow trans exit). The constriction is typically a narrowing in the channel which runs through the nanopore which may determine or control the signal obtained when the substrate (e.g. target substrate) moves with respect to the nanopore.

In some embodiments, an internal diameter (e.g., internal lumen diameter) can be at least about 5 nm, at least about 6 nm, at least about 7 nm, at least about 8 nm, at least about 9 nm, at least about 10 nm, at least about 11 nm, at least about 12 nm, at least about 13 nm, at least about 14 nm, at least about 15 nm, at least about 16 nm, at least about 17 nm, at least about 18 nm, at least about 19 nm, at least about 20 nm, or greater than about 20 nm. In some embodiments, an internal diameter (e.g., internal lumen diameter) can be at most about 20 nm, at most about 19 nm, at most about 18 nm, at most about 17 nm, at most about 16 nm, at most about 15 nm, at most about 14 nm, at most about 13 nm, at most about 12 nm, at most about 11 nm, at most about 10 nm, at most about 9 nm, at most about 8 nm, at most about 7 nm, at most about 6 nm, at most about 5 nm, or less than about 5 nm.

In some embodiments, an internal diameter (e.g., an internal lumen diameter) can be from about 5 nm to about 25 nm. In some embodiments, an internal diameter (e.g., internal lumen diameter) can be from about 5 nm to about 6 nm, about 5 nm to about 7 nm, about 5 nm to about 8 nm, about 5 nm to about 9 nm, about 5 nm to about 10 nm, about 5 nm to about 12 nm, about 5 nm to about 14 nm, about 5 nm to about 16 nm, about 5 nm to about 18 nm, about 5 nm to about 20 nm, about 5 nm to about 25 nm, about 6 nm to about 7 nm, about 6 nm to about 8 nm, about 6 nm to about 9 nm, about 6 nm to about 10 nm, about 6 nm to about 12 nm, about 6 nm to about 14 nm, about 6 nm to about 16 nm, about 6 nm to about 18 nm, about 6 nm to about 20 nm, about 6 nm to about 25 nm, about 7 nm to about 8 nm, about 7 nm to about 9 nm, about 7 nm to about 10 nm, about 7 nm to about 12 nm, about 7 nm to about 14 nm, about 7 nm to about 16 nm, about 7 nm to about 18 nm, about 7 nm to about 20 nm, about 7 nm to about 25 nm, about 8 nm to about 9 nm, about 8 nm to about 10 nm, about 8 nm to about 12 nm, about 8 nm to about 14 nm, about 8 nm to about 16 nm, about 8 nm to about 18 nm, about 8 nm to about 20 nm, about 8 nm to about 25 nm, about 9 nm to about 10 nm, about 9 nm to about 12 nm, about 9 nm to about 14 nm, about 9 nm to about 16 nm, about 9 nm to about 18 nm, about 9 nm to about 20 nm, about 9 nm to about 25 nm, about 10 nm to about 12 nm, about 10 nm to about 14 nm, about 10 nm to about 16 nm, about 10 nm to about 18 nm, about 10 nm to about 20 nm, about 10 nm to about 25 nm, about 12 nm to about 14 nm, about 12 nm to about 16 nm, about 12 nm to about 18 nm, about 12 nm to about 20 nm, about 12 nm to about 25 nm, about 14 nm to about 16 nm, about 14 nm to about 18 nm, about 14 nm to about 20 nm, about 14 nm to about 25 nm, about 16 nm to about 18 nm, about 16 nm to about 20 nm, about 16 nm to about 25 nm, about 18 nm to about 20 nm, about 18 nm to about 25 nm, or about 20 nm to about 25 nm.

In some embodiments, an internal diameter (e.g., internal lumen diameter) can be about 5 nm, about 6 nm, about 7 nm, about 8 nm, about 9 nm, about 10 nm, about 11 nm, about 12 nm, about 13 nm, about 14 nm, about 15 nm, about 16 nm, about 17 nm, about 18 nm, about 19 nm, or about 20 nm.

In some embodiments, the nanopore (e.g., biological nanopore) may be a pore-forming toxin. Pore-forming toxins (PFTs) of pathogenic bacteria are well-characterized virulence factors. They belong to an ancient and largely diverse protein family. PFTs are found across Gram-negative and -positive clades of bacteria, with members amongst human, insect and plant pathogens. Depending on the secondary structural nature of the membrane perforating channel, PFTs are divided into two families: α-PFTs form α-helical pores, while β-PFTs produce β-barrel pores.

In one aspect, the nanopore is a member of the cytolysin A (ClyA) family of toxins, or a mutant thereof enabling site specific functionalization with a recognition element (e.g., proteinaceous recognition element). ClyA-like toxins include PDB ID: 1QOY (soluble ClyA), 2WCD (protomeric ClyA), 6EK7 (soluble YaxA), 6EL1 (protomeric YaxA, protomeric YaxB), 6EK4 (soluble PaxB), 4KIP (soluble NheA), 5KUC (Cry6AA), 2NRJ (Hbl-B) See Bräuning et al., which is incorporated by reference herein in its entirety, (Toxins (Basel). 2018 September; 10 (9): 343, which is incorporated by reference herein in its entirety) and references cited therein, which are incorporated by reference herein in its entirety.

In some embodiments, the nanopore is ClyA, preferably a mutant ClyA, that is functionalized with R in the region comprising amino acids F101 to S110 as found in the GenBank sequence AJ313032.1. ClyA is an α-helical PFT with a comparatively large diameter (3-6 nm, depending on the entrance).

In one aspect, the modified nanopore is based on the ClyA variant ClyA-AS, having the sequence

MTGIFAEQTVEVVKSAIETADGALDLYNKYLDQVIPWKTFDETIKELSRFKQEYS QEASVLVGDIKVLLMDSQDKYFEATQTVYEWAGVVTQLLSAYIQLFDGYNEKKASAQKDI LIRILDDGVKKLNEAQKSLLTSSQSFNNASGKLLALDSQLTNDFSEKSSYYQSQVDRIRKEA YAGAAAGIVAGPFGLIISYSIAAGVVEGKLIPELNNRLKTVQNFFTSLSATVKQANKDIDAAK LKLATEIAAIGEIKTETETTRFYVDYDDLMLSLLKGAAKKMINTSNEYQQRHGRKTLFEVPD VGSSYHHHHH (SEQ ID NO: 1). This variant contains the following mutations relative to the wild-type ClyA protein: C87A, L99Q, E103G, F166Y, I203V, C285S, K294R. To allow for R functionalization, the further mutation S110C is introduced.

In some embodiments, suitable water facing amino acids in the vicinity of the entrance (e.g., cis entrance) of the nanopore can be modified from structural models. In some examples, other useful regions for ClyA functionalization include residues D267-S272, the lumen-exposed residues in the helix comprising A111-Q139 and the helix comprising D71-D64. Preferred, non-limiting examples of residues for modification include D114, E129, K132, S133, V136, Q139, E78, D71 and D64. ClyA residues on the outside of the ClyA nanopore to be modified include those from S272-until the end of the protein, from F101 to K66, and D267-K230.

In one aspect, ClyA is suitably functionalized at position 110 by using mutant S110C.

In one aspect, the nanopore is a modified member of the family of YaxAB The Yersinia YaxAB system represents a family of binary α-PFTs with orthologues in human, insect, and plant pathogens.

In some embodiments, the nanopore is Pleurotus ostreatus pleurotolysin (PlyAB; PDB ID 4V2T), or a mutant thereof enabling site specific functionalization with a recognition element (e.g., proteinaceous recognition element). PlyAB consists of two distinct components: Pleurotolysin A (PlyA, 16 kDa) acts as a scaffold to recruit the second component pleurotolysin B (PlyB, 54 kDa), which spans the lipid bilayer. Cryogenic electron microscopy revealed a nanopore with an entry (e.g., a cis entry) of ˜10.5 nm, an entry (e.g., a trans entry) of ˜7.2 nm, and a constriction with a diameter of ˜5.5 nm. Suitable regions for attaching R include S49 to D71, R100 to S89, G181 to G205, D298 to E316, and V329 to P336, based on the residue numbering of AJ313032.1.

In some embodiments, the concept of analyte-dependent dynamics of recognition element R moving in/out the lumen of a nanopore is also advantageously applied to de novo made nanopores such as DNA-based membrane nanopores of tunable pore shapes and lumen widths of up to tens of nanometers (Xingh et al. 2022, Nature Nanotechnology vol. 17, pg. 708-713, which is incorporated by reference herein in its entirety), or DNA origami nanopores that have an inner diameter as large as 30 nm (Fragrasso et al. ACS Nano 2021, 15, 8, 12768-12779, which is incorporated by reference herein in its entirety). Still further, the nanopore is a de novo nanopore based on de novo alpha-helical or beta-barrel transmembrane regions (see e.g. Shimizu et al. 2022, Nature Nanotechnology volume 17, pg. 67-75, which is incorporated by reference herein in its entirety; or Scott et al. 2021, Nature Chemistry volume, 13, pg. 643-650, which is incorporated by reference herein in its entirety; or Vorobieva et al. 2021, Science, Vol 371, Issue 6531, which is incorporated by reference herein in its entirety)).

Accordingly, the invention also provides methods and sensor systems comprising an artificial, non-solid state nanopore that is functionalized with a 5 to 50 kDa recognition element (e.g., proteinaceous recognition element) R capable of specifically binding to an analyte (e.g., target analyte) and wherein R dynamically moves in and out of the interior of the nanopore to provoke transient current blockage events, and wherein binding of R to the analyte (e.g., target analyte, e.g., target protein), modulates its dynamic movement, thereby inducing a change in the frequency and/or magnitude of the current blockage events, wherein the change in the frequency and/or magnitude of current blockage events indicates the presence of the analyte (e.g., target analyte) in the sample. In one embodiment, it comprises a nanobody-functionalized DNA-based membrane nanopore or DNA origami nanopore.

In some embodiments, a nanopore (e.g., artificial or non-solid state nanopore) is coupled to an at least about 1 kDa, at least about 2 kDa, at least about 3 kDa, at least about 4 kDa, at least about 5 kDa, at least about 6 kDa, at least about 7 kDa, at least about 8 kDa, at least about 9 kDa, at least about 10 kDa, at least about 11 kDa, at least about 12 kDa, at least about 13 kDa, at least about 14 kDa, at least about 15 kDa, at least about 16 kDa, at least about 17 kDa, at least about 18 kDa, at least about 19 kDa, at least about 20 kDa, at least about 21 kDa, at least about 22 kDa, at least about 23 kDa, at least about 24 kDa, at least about 25 kDa, at least about 26 kDa, at least about 27 kDa, at least about 28 kDa, at least about 29 kDa, at least about 30 kDa, at least about 31 kDa, at least about 32 kDa, at least about 33 kDa, at least about 34 kDa, at least about 35 kDa, at least about 36 kDa, at least about 37 kDa, at least about 38 kDa, at least about 39 kDa, at least about 40 kDa, at least about 41 kDa, at least about 42 kDa, at least about 43 kDa, at least about 44 kDa, at least about 45 kDa, at least about 46 kDa, at least about 47 kDa, at least about 48 kDa, at least about 49 kDa, at least about 50 kDa, or greater than about 50 kDa recognition element capable of specifically binding to an analyte (e.g., target analyte). In some embodiments, a nanopore (e.g., artificial or non-solid state nanopore) is coupled to an at most about 50 kDa, at most about 49 kDa, at most about 48 kDa, at most about 47 kDa, at most about 46 kDa, at most about 45 kDa, at most about 44 kDa, at most about 43 kDa, at most about 42 kDa, at most about 41 kDa, at most about 40 kDa, at most about 39 kDa, at most about 38 kDa, at most about 37 kDa, at most about 36 kDa, at most about 35 kDa, at most about 34 kDa, at most about 33 kDa, at most about 32 kDa, at most about 31 kDa, at most about 30 kDa, at most about 29 kDa, at most about 28 kDa, at most about 27 kDa, at most about 26 kDa, at most about 25 kDa, at most about 24 kDa, at most about 23 kDa, at most about 22 kDa, at most about 21 kDa, at most about 20 kDa, at most about 19 kDa, at most about 18 kDa, at most about 17 kDa, at most about 16 kDa, at most about 15 kDa, at most about 14 kDa, at most about 13 kDa, at most about 12 kDa, at most about 11 kDa, at most about 10 kDa, at most about 9 kDa, at most about 8 kDa, at mots about 7 kDa, at most about 6 kDa, at most about 5 kDa, at most about 4 kDa, at most about 3 kDa, at most about 2 kDa, at most about 1 kDa, or less than about 1 kDa recognition element capable of specifically binding to an analyte.

In some embodiments, a nanopore (e.g., artificial or non-solid state nanopore) is coupled to an about 5 kDa to about 60 kDa recognition element capable of specifically binding to an analyte. In some embodiments, a nanopore (e.g., artificial or non-solid state nanopore) is coupled to an about 5 kDa to about 10 kDa, about 5 kDa to about 15 kDa, about 5 kDa to about 20 kDa, about 5 kDa to about 25 kDa, about 5 kDa to about 30 kDa, about 5 kDa to about 35 kDa, about 5 kDa to about 40 kDa, about 5 kDa to about 45 kDa, about 5 kDa to about 50 kDa, about 5 kDa to about 55 kDa, about 5 kDa to about 60 kDa, about 10 kDa to about 15 kDa, about 10 kDa to about 20 kDa, about 10 kDa to about 25 kDa, about 10 kDa to about 30 kDa, about 10 kDa to about 35 kDa, about 10 kDa to about 40 kDa, about 10 kDa to about 45 kDa, about 10 kDa to about 50 kDa, about 10 kDa to about 55 kDa, about 10 kDa to about 60 kDa, about 15 kDa to about 20 kDa, about 15 kDa to about 25 kDa, about 15 kDa to about 30 kDa, about 15 kDa to about 35 kDa, about 15 kDa to about 40 kDa, about 15 kDa to about 45 kDa, about 15 kDa to about 50 kDa, about 15 kDa to about 55 kDa, about 15 kDa to about 60 kDa, about 20 kDa to about 25 kDa, about 20 kDa to about 30 kDa, about 20 kDa to about 35 kDa, about 20 kDa to about 40 kDa, about 20 kDa to about 45 kDa, about 20 kDa to about 50 kDa, about 20 kDa to about 55 kDa, about 20 kDa to about 60 kDa, about 25 kDa to about 30 kDa, about 25 kDa to about 35 kDa, about 25 kDa to about 40 kDa, about 25 kDa to about 45 kDa, about 25 kDa to about 50 kDa, about 25 kDa to about 55 kDa, about 25 kDa to about 60 kDa, about 30 kDa to about 35 kDa, about 30 kDa to about 40 kDa, about 30 kDa to about 45 kDa, about 30 kDa to about 50 kDa, about 30 kDa to about 55 kDa, about 30 kDa to about 60 kDa, about 35 kDa to about 40 kDa, about 35 kDa to about 45 kDa, about 35 kDa to about 50 kDa, about 35 kDa to about 55 kDa, about 35 kDa to about 60 kDa, about 40 kDa to about 45 kDa, about 40 kDa to about 50 kDa, about 40 kDa to about 55 kDa, about 40 kDa to about 60 kDa, about 45 kDa to about 50 kDa, about 45 kDa to about 55 kDa, about 45 kDa to about 60 kDa, about 50 kDa to about 55 kDa, about 50 kDa to about 60 kDa, or about 55 kDa to about 60 kDa recognition element capable of specifically binding to an analyte.

In some embodiments, a nanopore (e.g., artificial or non-solid state nanopore) is coupled to an about 1 kDa, about 2 kDa, about 3 kDa, about 4 kDa, about 5 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa, about 17 kDa, about 18 kDa, about 19 kDa, about 20 kDa, about 21 kDa, about 22 kDa, about 23 kDa, about 24 kDa, about 25 kDa, about 26 kDa, about 27 kDa, about 28 kDa, about 29 kDa, about 30 kDa, about 31 kDa, about 32 kDa, about 33 kDa, about 34 kDa, about 35 kDa, about 36 kDa, about 37 kDa, about 38 kDa, about 39 kDa, about 40 kDa, about 41 kDa, about 42 kDa, about 43 kDa, about 44 kDa, about 45 kDa, about 46 kDa, about 47 kDa, about 48 kDa, about 49 kDa, or about 50 kDa recognition element capable of specifically binding to an analyte.

### Analytes

In some embodiments, a method or sensor system of the invention can be readily designed to detect any analyte (e.g. target analyte) or multiple analytes (e.g. multiple target analytes) of interest. The invention is advantageously used to detect a label-free analyte (e.g., target analyte).

In one embodiment, the invention provides a method for detecting an analyte/antigen (e.g. target analyte/antigen) selected from the group consisting of a protein, polypeptide, a protein assembly, a protein/DNA assembly, polysaccharide, lipid, lipid membrane, lipid particle, bacterium, virus capsid, virus particle, dendrimer, polymer, or any combination thereof.

In some cases, the analyte (e.g. target analyte) is a protein. In some instances, the protein (e.g. target protein) is selected from the group consisting of a folded/native protein, a clinically relevant protein, a protein biomarker, a pathogenic protein, a cell surface protein.

The present invention is particularly suitable for detecting protein targets covering a very wide range of masses and dimensions. In one aspect, the present invention detects proteins (e.g. protein targets) covering a very wide range of masses and dimensions, from very small proteins and peptides to very large proteins and complexes. Since the recognition elements R are dynamic, moving in and out of the nanopore, the system and methods are sensitive to R binding to both very small analytes and to very large analytes that cannot fit inside the nanopore.

In some embodiments, the present invention is particularly suitable for detecting analytes or analyte (e.g. protein analytes or protein analyte) complexes that are larger than 50 Da, preferably larger than 100 Da, most preferably larger than 150 Da.

In some embodiments, the analytes or analyte complexes are at least about 25 kDa, at least about 50 kDa, at least about 60 kDa, at least about 70 kDa, at least about 80 kDa, at least about 90 kDa, at least about 100 kDa, at least about 110 kDa, at least about 120 kDa, at least about 130 kDa, at least about 140 kDa, at least about 150 kDa, at least about 175 kDa, at least about 200 kDa, at least about 250 kDa, at least about 300 kDa, at least about 400 kDa, at least about 500 kDa, at least about 750 kDa, at least about 1,000 kDa, or greater than about 1,000 kDa. In some embodiments, the analytes or analyte complexes are at most about 1,000 kDa, at most about 750 kDa, at most about 500 kDa, at most about 400 kDa, at most about 300 kDa, at most about 250 kDa, at most about 200 kDa, at most about 175 kDa, at most about 150 kDa, at most about 140 kDa, at most about 130 kDa, at most about 120 kDa, at most about 110 kDa, at most about 100 kDa, at most about 90 kDa, at most about 80 kDa, at most about 70 kDa, at most about 60 kDa, at most about 50 kDa, at most about 25 kDa, or less than about 25 kDa.

In some embodiments, the analytes or analyte complexes are from about 50 kDa to about 500 kDa. In some embodiments, the analytes or analyte complexes are from about 50 kDa to about 60 kDa, about 50 kDa to about 70 kDa, about 50 kDa to about 80 kDa, about 50 kDa to about 90 kDa, about 50 kDa to about 100 kDa, about 50 kDa to about 125 kDa, about 50 kDa to about 150 kDa, about 50 kDa to about 175 kDa, about 50 kDa to about 200 kDa, about 50 kDa to about 250 kDa, about 50 kDa to about 500 kDa, about 60 kDa to about 70 kDa, about 60 kDa to about 80 kDa, about 60 kDa to about 90 kDa, about 60 kDa to about 100 kDa, about 60 kDa to about 125 kDa, about 60 kDa to about 150 kDa, about 60 kDa to about 175 kDa, about 60 kDa to about 200 kDa, about 60 kDa to about 250 kDa, about 60 kDa to about 500 kDa, about 70 kDa to about 80 kDa, about 70 kDa to about 90 kDa, about 70 kDa to about 100 kDa, about 70 kDa to about 125 kDa, about 70 kDa to about 150 kDa, about 70 kDa to about 175 kDa, about 70 kDa to about 200 kDa, about 70 kDa to about 250 kDa, about 70 kDa to about 500 kDa, about 80 kDa to about 90 kDa, about 80 kDa to about 100 kDa, about 80 kDa to about 125 kDa, about 80 kDa to about 150 kDa, about 80 kDa to about 175 kDa, about 80 kDa to about 200 kDa, about 80 kDa to about 250 kDa, about 80 kDa to about 500 kDa, about 90 kDa to about 100 kDa, about 90 kDa to about 125 kDa, about 90 kDa to about 150 kDa, about 90 kDa to about 175 kDa, about 90 kDa to about 200 kDa, about 90 kDa to about 250 kDa, about 90 kDa to about 500 kDa, about 100 kDa to about 125 kDa, about 100 kDa to about 150 kDa, about 100 kDa to about 175 kDa, about 100 kDa to about 200 kDa, about 100 kDa to about 250 kDa, about 100 kDa to about 500 kDa, about 125 kDa to about 150 kDa, about 125 kDa to about 175 kDa, about 125 kDa to about 200 kDa, about 125 kDa to about 250 kDa, about 125 kDa to about 500 kDa, about 150 kDa to about 175 kDa, about 150 kDa to about 200 kDa, about 150 kDa to about 250 kDa, about 150 kDa to about 500 kDa, about 175 kDa to about 200 kDa, about 175 kDa to about 250 kDa, about 175 kDa to about 500 kDa, about 200 kDa to about 250 kDa, about 200 kDa to about 500 kDa, or about 250 kDa to about 500 kDa.

In some embodiments, the analytes or analyte complexes are about 25 kDa, about 50 kDa, about 60 kDa, about 70 kDa, about 80 kDa, about 90 kDa, about 100 kDa, about 110 kDa, about 120 kDa, about 130 kDa, about 140 kDa, about 150 kDa, about 175 kDa, about 200 kDa, about 250 kDa, about 300 kDa, about 400 kDa, about 500 kDa, about 750 kDa, or about 1,000 kDa.

The methods and systems are capable of detecting analytes far too large accommodated even inside very large nanopores. In some embodiments, the invention is broadly capable of binding and detecting a much wider variety of biologically relevant biomarkers, such as large proteins, protein complexes, including intact viruses, bacterium and cells.

In some embodiments, the disclosed methods are for detecting or characterising modifications in an analyte (e.g., analyte, e.g., target protein, e.g., label-free target protein). In one aspect, one or more of the amino acids/derivatives/analogs in the target protein is post-translationally modified. Any one or more post-translational modifications may be present in the protein (e.g. target protein).

In some embodiments, post-translational modifications include modification with a hydrophobic group, modification with a cofactor, addition of a chemical group, glycation (the non-enzymatic attachment of a sugar), biotinylation and PEGylation. Post-translational modifications can also be non-natural, for instance are chemical modifications introduced in a laboratory for biotechnological or biomedical purposes. This allows for monitoring the levels of post-translational modifications of the laboratory-derived peptide, polypeptide or protein as compared to the natural counterpart. As such, the methods disclosed herein can be used to detect the presence, absence, extent or number of positions of post-translational modifications in a polypeptide.

In some embodiments, post-translational modification with a hydrophobic group can include myristoylation, palmitoylation, isoprenylation or prenylation, the attachment of an isoprenoid group; farnesylation, the attachment of a farnesol group; geranylgeranylation, the attachment of a geranylgeraniol group; and glypiation, and glycosylphosphatidylinositol (GPI) anchor formation via an amide bond. Examples of post-translational modification with a cofactor include lipoylation, attachment of a lipoate (Cs) functional group; flavination, attachment of a flavin moiety (e.g. flavin mononucleotide (FMN) or flavin adenine dinucleotide (FAD)); attachment of heme C, for instance via a thioether bond with cysteine; phosphopantetheinylation, the attachment of a 4′-phosphopantetheinyl group; retinylidene Schiff base formation; or any combination thereof.

In some embodiments, post-translational modification by addition of a chemical group can include acylation, e.g. O-acylation (esters), N-acylation (amides) or S-acylation (thioesters); acetylation, the attachment of an acetyl group for instance to the N-terminus or to lysine; formylation; alkylation, the addition of an alkyl group, such as methyl or ethyl; methylation, the addition of a methyl group for instance to lysine or arginine; amidation; butyrylation; gamma-carboxylation; glycosylation, the enzymatic attachment of a glycosyl group for instance to arginine, asparagine, cysteine, hydroxylysine, serine, threonine, tyrosine or tryptophan; polysialylation, the attachment of polysialic acid; malonylation; hydroxylation; iodination; bromination; citrulination; nucleotide addition, the attachment of any nucleotide such as any of those discussed above, ADP ribosylation; oxidation; phosphorylation, the attachment of a phosphate group for instance to serine, threonine or tyrosine (O-linked) or histidine (N-linked); adenylylation, the attachment of an adenylyl moiety for instance to tyrosine (O-linked) or to histidine or lysine (N-linked); propionylation; pyroglutamate formation; S-glutathionylation; Sumoylation; S-nitrosylation; succinylation, the attachment of a succinyl group for instance to lysine; selenoylation, the incorporation of selenium; ubiquitinilation, the addition of ubiquitin subunits (N-linked); or any combination thereof.

### Nanopore Sensor System

A further embodiment relates to a nanopore system, comprising a fluid-filled compartment separated by a membrane into a first chamber and a second chamber, electrodes capable of applying a potential across the membrane, one or more functionalized nanopores according to the invention inserted in the membrane.

In one aspect, the invention provides a nanopore system comprising a membrane having a modified nanopore therein, the membrane separating a fluid chamber into a first side and a second side, and means for providing a voltage difference between the first side and the second side of the membrane, wherein the modified nanopore is a biological or de novo formed non-solid state nanopore that is functionalized with a 5 to 50 kDa, preferably 10 to 40 kDa, more preferably 12-15 kDa, recognition element capable of specifically binding to an analyte (e.g. target analyte).

In some embodiments, a biological or de novo formed non-solid state nanopore is coupled an at least about 5 kDa, at least about 10 kDa, at least about 15 kDa, at least about 20 kDa, at least about 25 kDa, at least about 30 kDa, at least about 35 kDa, at least about 40 kDa, at least about 45 kDa, at least about 50 kDa, or greater than about 50 kDa recognition element capable of specifically binding to an analyte. In some embodiments, a biological or de novo formed non-solid state nanopore is coupled an at most about 50 kDa, at most about 45 kDa, at most about 40 kDa, at most about 35 kDa, at most about 30 kDa, at most about 25 kDa, at most about 20 kDa, at most about 15 kDa, at most about 10 kDa, at most about 5 kDa, or less than about 5 kDa recognition element capable of specifically binding to an analyte.

In some embodiments, a biological or de novo formed non-solid state nanopore is coupled an about 5 kDa to about 60 kDa recognition element capable of specifically binding to an analyte. In some embodiments, a biological or de novo formed non-solid state nanopore is coupled an about 5 kDa to about 10 kDa, about 5 kDa to about 15 kDa, about 5 kDa to about 20 kDa, about 5 kDa to about 25 kDa, about 5 kDa to about 30 kDa, about 5 kDa to about 35 kDa, about 5 kDa to about 40 kDa, about 5 kDa to about 45 kDa, about 5 kDa to about 50 kDa, about 5 kDa to about 55 kDa, about 5 kDa to about 60 kDa, about 10 kDa to about 15 kDa, about 10 kDa to about 20 kDa, about 10 kDa to about 25 kDa, about 10 kDa to about 30 kDa, about 10 kDa to about 35 kDa, about 10 kDa to about 40 kDa, about 10 kDa to about 45 kDa, about 10 kDa to about 50 kDa, about 10 kDa to about 55 kDa, about 10 kDa to about 60 kDa, about 15 kDa to about 20 kDa, about 15 kDa to about 25 kDa, about 15 kDa to about 30 kDa, about 15 kDa to about 35 kDa, about 15 kDa to about 40 kDa, about 15 kDa to about 45 kDa, about 15 kDa to about 50 kDa, about 15 kDa to about 55 kDa, about 15 kDa to about 60 kDa, about 20 kDa to about 25 kDa, about 20 kDa to about 30 kDa, about 20 kDa to about 35 kDa, about 20 kDa to about 40 kDa, about 20 kDa to about 45 kDa, about 20 kDa to about 50 kDa, about 20 kDa to about 55 kDa, about 20 kDa to about 60 kDa, about 25 kDa to about 30 kDa, about 25 kDa to about 35 kDa, about 25 kDa to about 40 kDa, about 25 kDa to about 45 kDa, about 25 kDa to about 50 kDa, about 25 kDa to about 55 kDa, about 25 kDa to about 60 kDa, about 30 kDa to about 35 kDa, about 30 kDa to about 40 kDa, about 30 kDa to about 45 kDa, about 30 kDa to about 50 kDa, about 30 kDa to about 55 kDa, about 30 kDa to about 60 kDa, about 35 kDa to about 40 kDa, about 35 kDa to about 45 kDa, about 35 kDa to about 50 kDa, about 35 kDa to about 55 kDa, about 35 kDa to about 60 kDa, about 40 kDa to about 45 kDa, about 40 kDa to about 50 kDa, about 40 kDa to about 55 kDa, about 40 kDa to about 60 kDa, about 45 kDa to about 50 kDa, about 45 kDa to about 55 kDa, about 45 kDa to about 60 kDa, about 50 kDa to about 55 kDa, about 50 kDa to about 60 kDa, or about 55 kDa to about 60 kDa recognition element capable of specifically binding to an analyte.

In some embodiments, a biological or de novo formed non-solid state nanopore is coupled to an about 5 kDa, about 10 kDa, about 15 kDa, about 20 kDa, about 25 kDa, about 30 kDa, about 35 kDa, about 40 kDa, or about 50 kDa recognition element capable of specifically binding to an analyte.

In some embodiments, the term “membrane” is used herein in its conventional sense to refer to a thin, film-like structure that separates the chamber of the system into a first side (e.g., a first compartment) or a cis side (or a cis compartment) and a second side (e.g, a second compartment) or a trans side (or a trans compartment) of the fluid chamber. The membrane separating the first side (or a cis side) and the second side (or trans side) comprises at least one R-functionalized nanopore. Membranes can be generally classified into synthetic membranes and biological membranes. Any membrane may be used in accordance with the invention. Multiple nanopores may be present in one membrane.

In some embodiments, suitable membranes are well-known in the art. In some cases, the membrane is an amphiphilic layer. An amphiphilic layer is a layer formed from amphiphilic molecules, such as phospholipids, which have both at least one hydrophilic portion and at least one lipophilic or hydrophobic portion. The amphiphilic layer may be a monolayer or a bilayer. The amphiphilic molecules may be synthetic or naturally occurring. Non-naturally occurring amphiphiles which form a monolayer are known in the art and include, for example, block copolymers (Gonzalez-Perez et al., Langmuir, 2009, 25, 10447-10450, which is incorporated by reference herein in its entirety).

In some embodiments, the nanopore system comprises a first side of a fluid chamber or cis chamber comprising a first conductive liquid medium in liquid communication with a second side of a fluid chamber or trans chamber comprising a second conductive liquid medium. The conductive liquid medium in the chambers of the nanopore system can have a wide range of ionic contents well known in the art, typically from 0.05 M to >3 M. In some embodiments, the conductive liquid medium can have an ionic content of at least about 0.01 M, at least about 0.05 M, at least about 0.1 M, at least about 0.5 M, at least about 1.0 M, at least about 1.5 M, at least about 2.0 M, at least about 2.5 M, at least about 3.0 M, at least about 3.5 M, at least about 4.0 M, at least about 4.5 M, at least about 5.0 M, or greater than about 5.0 M. In some embodiments, the conductive liquid medium can have an ionic content of at most about 5.0 M, at most about 4.5 M, at most about 4.0 M, at most about 3.5 M, at most about 3.0 M, at most about 2.5 M, at most about 2.0 M, at most about 1.5 M, at most about 1.0 M, at most about 0.5 M, at most about 0.1 M, at most about 0.05 M, at most about 0.01 M, or less than about 0.01 M.

In some embodiments, the conductive liquid medium can have an ionic content from about 0.01 M to about 5 M. In some embodiments, the conductive liquid medium can have an ionic content from about 0.01 M to about 0.05 M, about 0.01 M to about 0.1 M, about 0.01 M to about 0.5 M, about 0.01 M to about 1 M, about 0.01 M to about 1.5 M, about 0.01 M to about 2 M, about 0.01 M to about 2.5 M, about 0.01 M to about 3 M, about 0.01 M to about 3.5 M, about 0.01 M to about 4 M, about 0.01 M to about 5 M, about 0.05 M to about 0.1 M, about 0.05 M to about 0.5 M, about 0.05 M to about 1 M, about 0.05 M to about 1.5 M, about 0.05 M to about 2 M, about 0.05 M to about 2.5 M, about 0.05 M to about 3 M, about 0.05 M to about 3.5 M, about 0.05 M to about 4 M, about 0.05 M to about 5 M, about 0.1 M to about 0.5 M, about 0.1 M to about 1 M, about 0.1 M to about 1.5 M, about 0.1 M to about 2 M, about 0.1 M to about 2.5 M, about 0.1 M to about 3 M, about 0.1 M to about 3.5 M, about 0.1 M to about 4 M, about 0.1 M to about 5 M, about 0.5 M to about 1 M, about 0.5 M to about 1.5 M, about 0.5 M to about 2 M, about 0.5 M to about 2.5 M, about 0.5 M to about 3 M, about 0.5 M to about 3.5 M, about 0.5 M to about 4 M, about 0.5 M to about 5 M, about 1 M to about 1.5 M, about 1 M to about 2 M, about 1 M to about 2.5 M, about 1 M to about 3 M, about 1 M to about 3.5 M, about 1 M to about 4 M, about 1 M to about 5 M, about 1.5 M to about 2 M, about 1.5 M to about 2.5 M, about 1.5 M to about 3 M, about 1.5 M to about 3.5 M, about 1.5 M to about 4 M, about 1.5 M to about 5 M, about 2 M to about 2.5 M, about 2 M to about 3 M, about 2 M to about 3.5 M, about 2 M to about 4 M, about 2 M to about 5 M, about 2.5 M to about 3 M, about 2.5 M to about 3.5 M, about 2.5 M to about 4 M, about 2.5 M to about 5 M, about 3 M to about 3.5 M, about 3 M to about 4 M, about 3 M to about 5 M, about 3.5 M to about 4 M, about 3.5 M to about 5 M, or about 4 M to about 5 M.

In some embodiments, the conductive liquid medium can have an ionic content of about 0.01 M, about 0.05 M, about 0.1 M, about 0.5 M, about 1.0 M, about 1.5 M, about 2.0 M, about 2.5 M, about 3.0 M, about 3.5 M, about 4.0 M, about 4.5 M, or about 5.0 M.

A wide range of salts can be used, such as NaCl and KCl. Suitable solutions include 150 mM NaCl, 50 mM Tris-HCl, pH 7.5. The first side and second side of the fluid chamber may be symmetric or asymmetric. The cis and trans chamber may be symmetric or asymmetric. A wide range of pH and temperature conditions can be used, for example in the range of pH 5-9, 10-50° C., preferably at about 37° C. In some embodiments, the pH of the solution can be at least about 3, at least about 3.5, at least about 4, at least about 4.5, at least about 5, at least about 5.5, at least about 6, at least about 6.5, at least about 7, at least about 7.5, at least about 8, at least about 8.5, at least about 9, at least about 9.5, at least about 10, or greater than about 10. In some embodiments, the pH of the solution can be at most about 10, at most about 9.5, at most about 9, at most about 8.5, at most about 8, at most about 7.5, at most about 7, at most about 6.5, at most about 6, at most about 5.5, at most about 5, at most about 4.5, at most about 4, at most about 3.5, at most about 3, or less than about 3.

In some embodiments, the pH of the solution can be from about 3 to about 10. In some embodiments, the pH of the solution can be from about 3 to about 4, about 3 to about 5, about 3 to about 5.5, about 3 to about 6, about 3 to about 6.5, about 3 to about 7, about 3 to about 7.5, about 3 to about 8, about 3 to about 8.5, about 3 to about 9, about 3 to about 10, about 4 to about 5, about 4 to about 5.5, about 4 to about 6, about 4 to about 6.5, about 4 to about 7, about 4 to about 7.5, about 4 to about 8, about 4 to about 8.5, about 4 to about 9, about 4 to about 10, about 5 to about 5.5, about 5 to about 6, about 5 to about 6.5, about 5 to about 7, about 5 to about 7.5, about 5 to about 8, about 5 to about 8.5, about 5 to about 9, about 5 to about 10, about 5.5 to about 6, about 5.5 to about 6.5, about 5.5 to about 7, about 5.5 to about 7.5, about 5.5 to about 8, about 5.5 to about 8.5, about 5.5 to about 9, about 5.5 to about 10, about 6 to about 6.5, about 6 to about 7, about 6 to about 7.5, about 6 to about 8, about 6 to about 8.5, about 6 to about 9, about 6 to about 10, about 6.5 to about 7, about 6.5 to about 7.5, about 6.5 to about 8, about 6.5 to about 8.5, about 6.5 to about 9, about 6.5 to about 10, about 7 to about 7.5, about 7 to about 8, about 7 to about 8.5, about 7 to about 9, about 7 to about 10, about 7.5 to about 8, about 7.5 to about 8.5, about 7.5 to about 9, about 7.5 to about 10, about 8 to about 8.5, about 8 to about 9, about 8 to about 10, about 8.5 to about 9, about 8.5 to about 10, or about 9 to about 10.

In some embodiments, the pH of the solution can be about 3, about 3.5, about 4, about 4.5, about 5, about 5.5, about 6, about 6.5, about 7, about 7.5, about 8, about 8.5, about 9, about 9.5, or about 10. In some embodiments, the temperature of the solution can be at least about 5° C., at least about 10° C., at least about 15° C., at least about 20° C., at least about 25° C., at least about 30° C., at least about 35° C., at least about 40° C., at least about 45° C., at least about 50° C., at least about 55° C., at least about 60° C., at least about 65° C., at least about 70° C., at least about 75° C., or greater than about 75° C. In some embodiments, the temperature of the solution can be at most about 75° C., at most about 70° C., at most about 65° C., at most about 60° C., at most about 55° C., at most about 50° C., at most about 45° C., at most about 40° C., at most about 35° C., at most about 30° C., at most about 25° C., at most about 20° C., at most about 15° C., at most about 10° C., at most about 5° C., or less than about 5° C.

In some embodiments, the temperature of the solution can be from about 5° C. to about 70° C. In some embodiments, the temperature of the solution can be from about 5° C. to about 10° C., about 5° C. to about 15° C., about 5° C. to about 20° C., about 5° C. to about 25° C., about 5° C. to about 30° C., about 5° C. to about 35° C., about 5° C. to about 40° C., about 5° C. to about 45° C., about 5° C. to about 50° C., about 5° C. to about 60° C., about 5° C. to about 70° C., about 10° C. to about 15° C., about 10° C. to about 20° C., about 10° C. to about 25° C., about 10° C. to about 30° C., about 10° C. to about 35° C., about 10° C. to about 40° C., about 10° C. to about 45° C., about 10° C. to about 50° C., about 10° C. to about 60° C., about 10° C. to about 70° C., about 15° C. to about 20° C., about 15° C. to about 25° C., about 15° C. to about 30° C., about 15° C. to about 35° C., about 15° C. to about 40° C., about 15° C. to about 45° C., about 15° C. to about 50° C., about 15° C. to about 60° C., about 15° C. to about 70° C., about 20° C. to about 25° C., about 20° C. to about 30° C., about 20° C. to about 35° C., about 20° C. to about 40° C., about 20° C. to about 45° C., about 20° C. to about 50° C., about 20° C. to about 60° C., about 20° C. to about 70° C., about 25° C. to about 30° C., about 25° C. to about 35° C., about 25° C. to about 40° C., about 25° C. to about 45° C., about 25° C. to about 50° C., about 25° C. to about 60° C., about 25° C. to about 70° C., about 30° C. to about 35° C., about 30° C. to about 40° C., about 30° C. to about 45° C., about 30° C. to about 50° C., about 30° C. to about 60° C., about 30° C. to about 70° C., about 35° C. to about 40° C., about 35° C. to about 45° C., about 35° C. to about 50° C., about 35° C. to about 60° C., about 35° C. to about 70° C., about 40° C. to about 45° C., about 40° C. to about 50° C., about 40° C. to about 60° C., about 40° C. to about 70° C., about 45° C. to about 50° C., about 45° C. to about 60° C., about 45° C. to about 70° C., about 50° C. to about 60° C., about 50° C. to about 70° C., or about 60° C. to about 70° C.

In some embodiments, the temperature of the solution can be about 5° C., about 10° C., about 15° C., about 20° C., about 25° C., about 30° C., about 35° C., about 40° C., about 45° C., about 50° C., about 55° C., about 60° C., about 65° C., about 70° C., about 75° C., or greater than about 75° C.

In some embodiments, the first side or cis chamber comprises a crowding or blocking agent that reduces unwanted nonspecific protein adsorption. In one embodiment, the blocking agent is BSA.

In some embodiments, the system may comprise a circuit that can both apply the voltage and measure the current. Alternatively, it comprises one circuit to apply the voltage difference and another to measure the current. It is also possible to create the voltage difference with an asymmetric salt across the membrane. For example, one of the chambers may contain a solution of high ionic strength.

In some embodiments, mechanisms for detecting the current between the first side (e.g., a cis side) and the second side (e.g., a trans side) were described in WO 00/79257 U.S. Pat. Application No. 6,46,594, 6,673, 6,673,615, 6,627,067, 6,464,842, 6,362,002, 6,267,872, 6,015,714, 6,428,959, 6,617,113 and 5,795,782 and US Publications Nos. 2004/0121525, 2003/0104428 and 2003/0104428. They may include electrodes directly associated with the channel or pore at or near the porous opening, electrodes placed within the first side and the second side (or cis and trans chambers), and insulated glass microelectrodes. Electrodes may be capable of, detecting differences in ionic current around two chambers or tunneling electrical current around the porous opening. In another configuration, the transport property is the flow of electrons around the diameter of the aperture which can be monitored by electrodes placed adjacent to or touching the circumference of the nanopore. Said electrodes can be attached to an Axopatch 200B amplifier to amplify a signal.

It is understood that acquisition systems described herein is not limited and that other systems for acquiring or measuring nanopore signals can be employed. Alternative electrical schemes can also be employed, on arrayed chip platforms for example, to achieve an equivalent voltage drop across the nanopore and/or membrane.

In some embodiments, the sensor system is advantageously integrated in a portable device comprising a plurality of sensor systems. For example, it is comprised in a point-of-care diagnostic medical devices, which are in vitro diagnostics used by health care professionals to obtain results rapidly near or at the site of a patient. These products can be useful to quickly determine a marker responsible for a certain disease, e.g., at a doctor's office or clinic.

### Array and Kit

The present disclosure provides an array comprising a multiplicity of sensor systems according to the invention. Preferably, the array comprises a multiplicity of discrete reservoirs, each of which comprises nanopores modified with one or more different R elements to allow for detection of different analytes.

In some embodiments, the array comprises nanopores that are pre-modified with an L moiety, thus allowing for end-user defined functionalization with one or more selected recognition elements (e.g., proteinaceous recognition elements R). For example, the L moiety of the pre-modified pore is selected to allow for the formation of a double-stranded nucleic acid between a oligonucleotide-conjugated nanobody or affimer or affibody of choice and a oligonucleotide-conjugated pore protein. In one embodiment, a system of the invention comprises an array of pre-modified pores that all have the same linker L oligonucleotide sequence, to which R binding partners (comprising a single species of R or a mixture of different R species) can be coupled with the appropriate complementary sequence so as to form the duplexes. In an alternative embodiment, an array of pre-modified nanopores comprises different L moieties that are specific to a set of complementary R sequences.

In one aspect, L is composed of an original strand and a complementary protector strand, allowing for R attachment to the pre-modified nanopores by toehold mediated strand displacement (TMSD).

Also provided are methods and kits for preparing such arrays.

Also provided herein is the use of a system or analytical device according to the invention for single molecule sensing analysis, preferably for sensing the presence or concentration of one or more analytes (e.g. target analytes) in a complex (clinically relevant) sample. In some embodiments, the invention provides the use of a method, nanopore or sensor system, array or kit, in single protein detection, preferably in combination with high throughput analysis.

In one aspect, the present disclosure provides an array comprising a plurality of nanopore systems according to any one of preceding embodiments. In some embodiments, the array comprises a multiplicity of discrete reservoirs. In some cases, one or more of the plurality of nanopore systems comprise nanopores modified with different recognition elements to allow for detection of different analytes.

In one aspect, the present disclosure provides a kit for preparing a system of any one of the preceding embodiments. In some embodiments, the kit comprises nanopore pre-modified with a linker. In some cases, the linker is part of a double-stranded DNA complex composed of the original strand and a complementary protector strand.

In one aspect, the present disclosure provides use of a method, nanopore, nanopore sensor system, array, or kit according to any one of preceding embodiments in single protein detection. In some embodiments, the single protein detection can be combined with high throughput analysis. In some embodiments, the sensor system is integrated in a portable device comprising a plurality of sensor systems.

### Computer Systems

The present disclosure provides computer systems that are programmed to implement methods of determining one or more characteristics of an analyte. FIG. 16 shows a computer system 1601 that is programmed or otherwise configured to determine presence or absence of an analyte. The computer system 1601 can regulate various aspects of detecting presence or absence of the analyte. The computer system 1601 can be an electronic device of a user or a computer system that is remotely located with respect to the electronic device. The electronic device can be a mobile electronic device.

The computer system 1601 includes a central processing unit (CPU, also “processor” and “computer processor” herein) 1605, which can be a single core or multi core processor, or a plurality of processors for parallel processing. The computer system 3001 also includes memory or memory location 1610 (e.g., random-access memory, read-only memory, flash memory), electronic storage unit 1615 (e.g., hard disk), communication interface 1620 (e.g., network adapter) for communicating with one or more other systems, and peripheral devices 1625, such as cache, other memory, data storage and/or electronic display adapters. The memory 1610, storage unit 1615, interface 1620 and peripheral devices 1625 are in communication with the CPU 1605 through a communication bus (solid lines), such as a motherboard. The storage unit 1615 can be a data storage unit (or data repository) for storing data. The computer system 1601 can be operatively coupled to a computer network (“network”) 1630 with the aid of the communication interface 1620. The network 1630 can be the Internet, an internet and/or extranet, or an intranet and/or extranet that is in communication with the Internet. The network 1630 in some cases is a telecommunication and/or data network. The network 1630 can include one or more computer servers, which can enable distributed computing, such as cloud computing. The network 1630, in some cases with the aid of the computer system 1601, can implement a peer-to-peer network, which may enable devices coupled to the computer system 1601 to behave as a client or a server.

The CPU 1605 can execute a sequence of machine-readable instructions, which can be embodied in a program or software. The instructions may be stored in a memory location, such as the memory 1610. The instructions can be directed to the CPU 1605, which can subsequently program or otherwise configure the CPU 1605 to implement methods of the present disclosure. Examples of operations performed by the CPU 1605 can include fetch, decode, execute, and writeback.

The CPU 1605 can be part of a circuit, such as an integrated circuit. One or more other components of the system 1601 can be included in the circuit. In some cases, the circuit is an application specific integrated circuit (ASIC).

The storage unit 1615 can store files, such as drivers, libraries and saved programs. The storage unit 1615 can store user data, e.g., user preferences and user programs. The computer system 1601 in some cases can include one or more additional data storage units that are external to the computer system 1601, such as located on a remote server that is in communication with the computer system 1601 through an intranet or the Internet.

The computer system 1601 can communicate with one or more remote computer systems through the network 1630. For instance, the computer system 1601 can communicate with a remote computer system of a user (e.g., a personal computer). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1601 via the network 1630.

Methods as described herein can be implemented by way of machine (e.g., computer processor) executable code stored on an electronic storage location of the computer system 1601, such as, for example, on the memory 1610 or electronic storage unit 1615. The machine executable or machine readable code can be provided in the form of software. During use, the code can be executed by the processor 1605. In some cases, the code can be retrieved from the storage unit 1615 and stored on the memory 1610 for ready access by the processor 1605. In some situations, the electronic storage unit 1615 can be precluded, and machine-executable instructions are stored on memory 1610.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1601, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1601 can include or be in communication with an electronic display 1635 that comprises a user interface (UI) 1640 for providing, for example, the identification of the analyte. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1605.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and the computer memory coupled thereto. The computer memory comprises machine executable code that, upon execution by the one or more computer processors, implements any of the methods above or elsewhere herein.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### LEGEND TO THE FIGURES

FIGS. 1A-1D. Attachment of a single-stranded DNA to ClyA nanopore. (FIG. 1A) Side view (left) and top view (right) of ClyA structure (PDB: 6mrt). Serine (colored purple) at position 110 was genetically mutated to cysteine to enable site-specific chemical modification. (FIG. 1B) Schematic model showing the conjugation strategy of attaching ssDNA to ClyA nanopore. A 16mer oligonucleotide, named f, was conjugated to ClyA monomer via a maleimide-PEG4-DBCO linker, where the maleimide reacted with —SH group on the protein and DBCO was clicked to the azide group on the oligo. ClyA-f monomer then oligomerized to ClyA-f oligomer in the presence of 0.2% DDM at 37° C. (FIG. 1C) SDS-PAGE analysis of the conjugation efficiency. Lane 1: protein ladder, lane 2: ClyA-S110C monomer, lane 3: after reaction of ClyA-S110C with maleimide-PEG4-DBCO (ClyA-DBCO), lane 4: after reaction of purified ClyA-DBCO with f-azide (ClyA-f). (FIG. 1D) Native polyacrylamide gel analysis of the oligomerization of ClyA-f. Lane 5: ClyA-f after oligomerization, lane 6: S110C mutated ClyA after oligomerization.

FIGS. 2A-2F. Functionalization of ClyA nanopore with Spike nanobody Ty1 and electrical characterization of the nanopore. (FIG. 2A) Schematic model showing the strategy of functionalizing ClyA nanopore with Ty1 nanobody, where Ty1-f′ was immobilized on ClyA-f nanopore by DNA strand hybridization. (FIG. 2B) I-V curves of ClyA-S110C (blue triangle), ClyA-f (black square) and ClyA-f-Ty1 (red circle) at applied potential ranging from −90 to 90 mV (three independent experiments). (FIG. 2C) Histogram showing conductance distribution of ClyA-f nanopore with (red) and without (black) Ty1 nanobody. (FIG. 2D) Representative current traces of ClyA-f-Ty1 under an applied potential of −20 mV. “in” and “out” represented the nanobodies locating inside (blocked pore) and outside (open pore) of the nanopore respectively. Iₒ is the open pore current and It is the blocked pore current. (FIG. 2E) All-point histogram of the current traces shown in D, which demonstrated well-defined distribution of the blockade signals. (FIG. 2F) Schematic model interpreting the reversible conformation change between blocked (left) and open (right) states of ClyA-f-Ty1 at applied potential of −20 mV, which corresponded to the movement of one of the Ty1 nanobodies in and out of the vestibule of the pore. All of the experiments were performed in 150 mM NaCl, 50 mM Tris-HCl, pH 7.5.

FIGS. 3A-3F. Single channel recording traces of ClyA-f-Ty1 and the analysis of residual current I_{b}/Iₒ, tᵢₙ and tₒᵤₜ under different applied potentials. (FIG. 3A) Representative current traces of ClyA-f-Ty1 under applied potentials ranging from −10 to −40 mV. (FIG. 3B) All-point histogram of current traces in A, showing Ty1 nanobodies tend to reside in ClyA nanopore with increasing applied potentials. (FIG. 3C, FIG. 3D) Histogram of logarithmic time of Ty1 locating inside and outside of ClyA, respectively. (FIG. 3E, FIG. 3F) The influence of applied potentials on the average logarithmic time of Ty1 locating inside and outside of ClyA. These experiments were performed in 150 mM NaCl, 50 mM Tris-HCl, pH 7.5.

FIGS. 4A-4B. Nanobody attachment to ClyA through DNA oligo hybridization verified using DNase I. (FIG. 4A) Current traces of ClyA-f-Ty1 before and after the addition of 5 U DNase I in the presence of 2.5 mM MgCl₂ at an applied potential of −20 mV. (FIG. 4B) Enlarged representative current traces from A, showing that the nanobodies attached to the ClyA nanopore were removed after the addition of DNase I after about 30 mins. All-point histograms were displayed on the top of the panel showing the current distribution before and after the addition of DNaseI. Schematic model shown above depicts how the nanobodies were removed from ClyA nanopore. The experiments were performed in 150 mM NaCl, 2.5 mM MgCl₂, 50 mM Tris-HCl, pH 7.5.

FIGS. 5A-5B. Detection of Spike protein by nanobody-functionalized nanopores. (FIG. 5A) Current traces of ClyA-f-Ty1 before and after the sequential addition of 6 μM BSA and 2.3 nM Spike protein. (FIG. 5B) Enlarged representative current traces from A (top), and all-point histograms of the current distribution (bottom). From left to right showed before and after the addition BSA and Spike proteins. The experiments were performed in 150 mM NaCl, 50 mM Tris-HCl, pH7.5.

FIGS. 6A-6G). Effect of BSA on nanobody internalization. (FIGS. 6A-6C) Histograms distribution of tₒᵤₜ before and after the addition of 3 μM BSA, or 6 μM BSA to the first side (e.g., a cis side) of a ClyA-f-Ty1 nanopore system. The histograms were fitted with single exponential function. (FIGS. 6D-6G) The change of blockade percentage, open percentage, average time of the coupled Ty1 staying inside the ClyA nanopore (tᵢₙ), average time of Ty1 staying outside the ClyA nanopore (tₒᵤₜ) with increasing concentration of BSA, respectively. (n=4, each experiment was conducted with independent nanopores. Error bars represent standard deviations). These experiments were performed in 150 mM NaCl, 50 mM Tris-HCl, pH 7.5.

FIGS. 7A-7E. The effect of adding Spike protein to the ClyA-f-Ty1 pore. (FIG. 7A) Current traces showed the transition of the pore from a dynamic state (alternating between Ty1 in the pore and out the pore) to a fully open state (with the Ty1 trapped outside the pore through binding to Spike protein) in the period immediately following the addition of 2.3 nM Spike protein. (FIG. 7B) current traces of ClyA-f-Ty1 in the period approximately 25 minutes post addition of 2.3 nM Spike protein. (FIG. 7C) All-point histogram of the current traces presented in FIG. 7B. (FIG. 7D, FIG. 7E) Histogram of the logarithm of tᵢₙ and tₒᵤₜ after the addition of 2.3 nM Spike protein in of the period about 25 minutes post addition of Spike protein. The histograms were fitted with the Gauss distribution function. These experiments were performed in 150 mM NaCl, 50 mM Tris-HCl, pH 7.5 with the presence of 6 μM BSA.

FIGS. 8A-8D. Open probability of ClyA-f-Ty1 correlates positively with Spike trimer protein concentration. (FIG. 8A) Representative current traces of ClyA-f-Ty1 before and after the addition of increasing concentration of Spike trimer protein. (FIG. 8B) All-point histograms were displayed to show the current distribution before and after the addition of increasing concentration of Spike protein. (FIG. 8C) Curve regression of the open probability in the function of spike concentrations. The curve was fitted by using Hill-Langmuir equation (n=1.31, K_{d}=760.6 pM). (FIG. 8D) The schematic model showing the dynamics of the interaction between ClyA-f-Ty1 and Spike protein. Ty1 nanobodies dynamically move in and out of ClyA nanopore under applied potential. Spike protein reversibly interact with the Ty1 nanobodies attached on the nanopore presumably in a multivalent fashion at high concentration of Spike trimers. The experiments were performed in 150 mM NaCl, 50 mM Tris-HCl, pH 7.5 in the presence of 6 μM BSA.

FIGS. 9A-9J. Influence of Spike proteins concentration on binding kinetics to the ClyA-f-Ty1 pore. (FIGS. 9A-9D) Histogram of logo (tₒᵤₜ) at Spike concentration of 115 pM, 230 pM, 345 pM, 460 pM, respectively. The data were fitted with Guass distribution. (FIGS. 9E-9H) Histogram of log₁₀(tᵢₙ) at Spike concentration of 115 pM, 230 pM, 345 pM, 460 pM, respectively. The data were fitted with Guass distribution. (FIG. 91, FIG. 9J) Concentration dependency of the logarithm of tₒᵤₜ and tᵢₙ. These experiments were performed in 150 mM NaCl, 50 mM Tris-HCl, pH 7.5 with the presence of 6 μM BSA.

FIGS. 10A-10H. Behavior of ClyA-f-Ty1 in the presence of blood. (FIG. 10A) Schematic model showing electrical measurement of ClyA-f-Ty1 in the presence of blood. (FIG. 10B) Current traces showing the current change before and after addition of 1 μL of blood to the ClyA-f-Ty1 nanopore presenting in 500 μL electrolyte buffer. (FIG. 10C, FIG. 10E) Representative current traces in the presence of 6 μM BSA (FIG. 10C) and after addition of 1 μL of blood (FIG. 10E). (FIG. 10D, FIG. 10F) All-point histogram of the current traces before (FIG. 10D) and after (FIG. 10F) addition of 1 μL of blood. (FIG. 10G) Histogram of the logarithm of dwell time in level0 before and after the addition of 1 μL of blood. (FIG. 10H) Histogram of the logarithm of dwell time in level 1 before and after the addition of 1 μL of blood. These experiments were performed in electrolyte buffer 150 mM NaCl, 50 mM Tris-HCl, pH 7.5 in the presence of 6 μM BSA.

FIGS. 11A-B. Detection of spike trimer in the presence of blood. (FIG. 11A, FIG. 11B) Representative current traces before (FIG. 11A) and after (FIG. 11B) the addition of 2.3 nM Spike protein in the presence of 1 μL blood at a bias of −20 mV. The experiment was performed in 150 mM NaCl, 50 mM Tris-HCl, pH 7.5 in the presence of 6 μM BSA.

FIGS. 12A-12B. Detection of Her2 with functionalized nanopores. (FIG. 12A) Representative current traces of ClyA attached by 2Rs15d nanobody (ClyA-f-15d) before and after the addition of 32.8 nM Her2 protein under an applied potential of −20 mV. (FIG. 12B) Representative current traces of ClyA attached by 2Rb17c nanobody (ClyA-f-17c) before and after the addition of 20.8 nM Her2 protein at the same applied potential. Reported binding affinity of 2Rs15d to Her2: kₒₙ=2.14×10⁵ M⁻¹ s⁻¹, K_{off}=5.71×10⁻⁴ s⁻¹, K_{D})=2.7 nM. Reported binding affinity of 2Rb17c to Her2: Kₒₙ=7.6×10⁶ M⁻¹ s⁻¹, K_{off}=4.58×10⁻² s⁻¹, K_{D}=6 nM. These experiments were performed in 150 mM NaCl, 50 mM Tris-HCl, pH 7.5 in the presence of 6 μM BSA.

FIGS. 13A-13E. Functionalized ClyA nanopore for the detection of muPA. (FIG. 13A) The crystal structure of muPA (purple) in complex with nb22 nanobody (green) (PDB: 5LHR). Reported binding affinity of nb22 to muPA56: kₒₙ=(4.6±0.8)×10⁵ M⁻¹ s⁻¹, k_{off}=(7.8±2.2)×10⁻⁵ s⁻¹, K_{D}=0.2±0.03 nM. (FIG. 13B) Representative current traces of ClyA-f-nb22 before and after adding 3 nM muPA under-15 mV applied potential. (FIG. 13C) Enlarged representative current traces after adding 3 nM muPA at −15 mV. The signals consisted of three blockade levels in addition to the open pore level (Level 0) with current blocking percentages of 13.7%±0.1% (Level 1), 34.1%±0.5% (Level 2), and 63.6%±0.1% (Level 3), respectively. (FIG. 13D) Heatmap of the blockade events observed after the addition of 3 nM muPA with the logarithm of the dwell time against current blockade percentage. (FIG. 13E) The schematic model showing the conformation changes of ClyA-f-nb22 in response to muPA proteins. The experiments were performed in 150 mM NaCl, 50 mM Tris-HCl, pH 7.5 with the presence of 6 μM BSA.

FIGS. 14A-14C. Schematic illustrations of some of the options for coupling targeting moieties R to nanopores N via the hybridization of duplexed oligonucleotide (e.g. dsDNA) linkers L (where one oligonucleotide strand of the duplex linker L is coupled to the nanopore N, and the other complementary strand is coupled to the binding moiety R). The figure illustrates 3 possible options for coupling the components. FIG. 14A) the N and R components are located at opposite ends of the duplex linker L. For example, this can easily be achieved by coupling the components to both 5′ ends of each strand, or to both 3′ ends. The distance “d” between the N and R coupling locations is principally controlled by the length of the duplexed oligonucleotide strands, and the flexibility of the system (which determines the ability of R to enter the nanopore) is in part dependent on the flexibility of duplex oligonucleotide (e.g. dsDNA), which is less flexible than single-strand oligonucleotide. FIG. 14B) the N and R components are located at the same end of the hybridized duplex linker L. E.g. one component is coupled to the 5′ end of strand 1 and the other to the 3′ end of strand 2, or vice versa. This method of coupling is favorable for positioning the coupling points of N and R closer together to reduce the distance “d”, while still allowing for much longer oligonucleotide strands if required. FIG. 14C) one or both the N and R components are coupled to the oligonucleotide strands of the hybridized duplex linker L at an internal position along the strand (figure only shows R coupled at a mid-point for simplicity), for example via coupling to the backbone or a base of the polynucleotide.

In all cases above, the oligonucleotides can have sections of non-duplexed single strand (e.g. ssDNA overhangs) to further control distances and optimize the flexibility of the coupled components.

FIGS. 15A-15D. Schematic illustrations of a nanopore N with a linker L that is initially in a protected state (FIG. 15A), comprising a hybridized protecting polynucleotide strand (i) that can be removed by applying voltage to the nanopore in a membrane system to capture and strip the protecting strand from the linker L (FIG. 15B). The deprotected nanopore (FIG. 15C) can then be combined with a selected binding moiety R which will hybridize to the linker L to create the functional N-L-R nanopore system (FIG. 15D).

FIG. 16. Depicts a computer system that is programmed or otherwise configured to implement methods provided herein.

### EXPERIMENTAL SECTION

### Materials

All the chemicals, except specifically stated, were purchased from Sigma-Aldrich. Unnatural amino acid (UAA) 4-azido-L-phenylalanine (pAzF) used in this study was synthesized in-house following a reported protocol⁵⁷. All the DNA oligos were purchased from IDT.

Expression and Purification of pAzF-Modified Nanobodies

The DNA encoding for Ty1⁵², nb22⁵⁵, 2Rs15d and 2Rb17c⁵⁴ nanobodies were cloned into PET22b (+) plasmid (Addgene) respectively, with a pelB leader sequence at the N-terminal and a hexahistidine tag (6×His) at the C-terminal. An amber stop codon (TAG) was added before the 6×His to incorporate UAA into the nanobody. The production of pAzF-modified nanobody was conducted by following an established protocol³⁸. Firstly, the constructed plasmid was transformed into BL21 E. coli cells. Cells were cultured in 1 L TB medium supplemented with 100 mL salt buffer (0.17 M KH₂PO₄, 0.72 M K²HPO⁴), 1 mL 2 M MgCl₂, 1 mL 100 mg/mL ampicillin, 1 mL 50 mg/ml spectinomycin, 10 mL 10% glucose and 250 mg 4-azido-L-phenylalanine at 37° C. at 200 rpm. When the OD600 reached at 0.6-0.9, IPTG with a final concentration of 1 mM was added. The protein induction was completed at 25° C. by overnight shaking. The cells were harvested by centrifuging at 4° C. and 4500 rpm for 15 min, which were then resuspended in 24 mL cold TES buffer (0.2 M Tris, pH 8, 0.5 mM EDTA, 0.5 M sucrose). The suspension was incubated at 4° C. and 200 rpm (horizontal rotator) for 6 h, followed by the addition of 48 mL ¼ TES buffer and incubation at 4° C. and 200 rpm for overnight. Subsequently, the cell suspension was centrifuged at 4° C. and 12000 g for 30 min. The supernatant was collected and supplemented with 5 mM MgCl₂, followed by a further purification by FPLC (GE Healthcare) using a 5 mL HisTrap column (GE Healthcare). Binding buffer and elution buffer used here were 20 mM and 500 mM imidazole, respectively, both were supplemented with 20 mM sodium phosphate pH 7.4, 500 mM NaCl. The purity of the protein was analysed on 4%-12% SDS-PAGE gel.

Conjugation of Nanobody with f′-Oligo

Oligo f′ ordered with amine group at 5′ end (NH2-C6-5′-ATCCGCGGGTGTCGGG-3′) (SEQ ID NO: 2) was firstly reacted with 20 fold excess of NHS-DBCO in 60% DMSO at pH 8.0 and 25° C. for overnight. After being purified by ethanol precipitation and subsequent reverse-phase HPLC, the DBCO-oligo was incubated with azide-modified nanobody in PBS at 25° C. for overnight. The reaction was optimized by adding different ratio of nanobody and f′-DBCO oligo. When the molar ratio was 5:1, the conjugation yield was above 70%. Therefore, this ratio was applied for the conjugation of all four nanobodies. Subsequently, the nanobody-f′ conjugates were purified by ion-exchange chromatography and verified by either 16% denaturing urea polyacrylamide gel electrophoresis or SDS-PAGE.

### Expression and Purification of ClyA-S110C Nanopore

The ClyA-S110C construct was prepared by mutating the serine on position 110 to a cysteine in the cysteine-free variant ClyA-CS as previously reported⁴¹. The constructed plasmid was transformed into E. coli BL21 (DE3) electrocompetent cell by electroporation. Cells were cultured in 2× YT medium containing 100 μg/mL ampicillin at 37° C. and 200 rpm until OD600 reaches 0.8-1. Protein expression was induced by adding 0.5 mM IPTG and incubating at 20° C. and 200 rpm for overnight. Cells were harvested by centrifugation at 6500 rpm and 4° C. for 15 min. The pellets were stored in −80° C. freezer for at least 1 h and then thawed at 37° C., followed by resuspension in 20 mL lysis buffer (10 mM imidazole pH 8.0, 150 mM NaCl, 50 mM Tris·HCl, pH 7.5, 1 mM MgCl₂, 5 mM TCEP) supplemented with 0.2 mg/mL of lysozyme. After incubating at 4° C. for 25 min on a rotator, the cells were further lysed by sonication. The lysate was then centrifuged at 6500 rpm and 4° C. for 30 min, and the supernatant was collected and incubated with Ni-NTA beads (Qiagen) at room temperature for 1 h on a rotator. Non-specific binding protein was removed by at least 20 column volumes of wash buffer (10 mM imidazole pH 8.0, 150 mM NaCl, 50 mM Tris. HCl, pH 7.5) and the protein was eluted from the beads in elution buffer (200 mM EDTA pH 7.5, 150 mM NaCl, 50 mM Tris. HCl, pH 7.5). The purity of the protein was analyzed on 4%-12% SDS-PAGE gel.

### Preparation of ClyA-f-Nb Nanopore

The freshly purified ClyA-S110C was firstly incubated with 20× molar excess of DBCO-PEG4-maleimide at pH of 7.5 and 4° C. overnight, gently shaking. Unreacted DBCO-PEG4-maleimide was removed in standard buffer (150 mM NaCl, 50 mM Tris·HCl, pH 7.5) using a 3 kDa cut-off Amicon filter (Millipore). The purified ClyA-PEG4-DBCO was then incubated with 1.5-fold excess of f-azide oligo, at 4° C. overnight, gently shaking, to create the ssDNA modified “ClyA-f” monomers. The f-azide oligo Linker was prepared by reaction of an oligo with an amino modification at the 5′ end (NH2-C6-5′-CCCGACACCCGCGGAT-3′) (SEQ ID NO: 3) with an azidobutyric acid NHS ester. SDS-PAGE gel was utilized to check the click reaction efficiency. The ClyA-f monomer was oligomerized in the presence of 0.2% n-dodecyl-β-D-maltoside (DDM) by incubating at 37° C. for 30 minutes. Subsequently, the oligomerized ClyA-S110C and ClyA-f were analyzed and purified by blue native polyacrylamide gel electrophoresis (BN-PAGE, Bio-Rad). Due to the negative charges of DNA oligos²⁹, ClyA-f oligomers migrated slightly faster than unmodified ClyA-S110C oligomers. According to the previous study⁴¹, the lowest oligomeric bands of ClyA-S110C and ClyA-f were type-I nanopores (12-mer). Therefore, ClyA-S110C and ClyA-f dodecamers were obtained by slicing these bands from the gel. After eluting from the gel pieces using 30 μL of the standard buffer with the presence of 0.02% DDM, the ClyA-f oligomers solution was aliquoted into 5 μL/tube. The concentration of ClyA-f dodecamers eluted from gel was too low to be measured by neither Nanodrop nor Bradford assay. Therefore, prior to the single-channel recording experiments, excess of nanobody-f′ (˜40 μmol) was incubated with 5 μL of the ClyA-f oligomers at room temperature for at least 30 mins to ensure each ClyA nanopore was maximally modified with duplexed nanobodies.

### Single-Channel Recording Experiment

Electrical recordings were performed using a vertical planar lipid membrane set-up as described previously⁵⁸ Briefly, a 1,2-diphytanoyl-sn-glycero-3-phosphocholine (DPhPC, purchased from Avanti Polar Lipids) lipid bilayer was formed on the aperture of the Teflon membrane separating the first side (e.g., cis side) and the second side (e.g., trans side) of the fluid chamber of a recording chamber. After being connected to a patch-clamp amplifier (Axopatch 200B, Axon Instruments) using Ag/AgCl electrodes, both the trans- and the first side of the chamber were filled with electrolyte buffer 150 mM NaCl, 50 mM Tris-HCl, pH 7.5. ClyA nanopores were added into the first side (e.g., cis side) of the chamber, which was connected to the ground electrode. After pore insertion, excess ClyA was removed by buffer exchange. DNaseI (Sigma-Aldrich), BSA, muPA (kindly provided by Emil Oldenburg), Her2 (acquired from SinoBiological) and various concentration of Spike proteins (SARS-CoV-2 S protein, purchased from ACROBiosystems) were all added to the first side (e.g., cis side) if not specifically stated. All recordings were conducted using a Bessel low-pass filter of 2 kHz and a sampling rate of 10 kHz. All electrical recording current traces were filtered by Gaussian lowpass filter with a cutoff of 1 kHz prior to analysis. The data analysis software we used in this study is Clampfit.

### Example 1: Functionalization of ClyA Nanopore with Nanobody

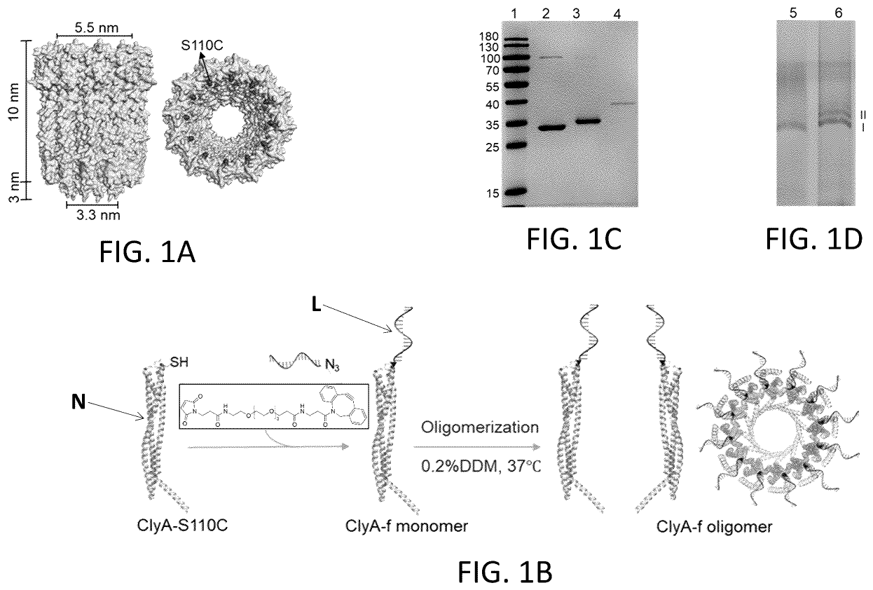

To specifically detect proteins with various sizes, we designed a ClyA nanopore functionalized with multiple nanobodies via a 16 base pair DNA duplex linker to the wide end of the pore. We hypothesized that the binding of proteins to the nanobodies would alter the ionic flux through the nanopore, thus inducing a distinguishable current signal indicating protein detection. To enable site-specific attachment of the DNA linker to ClyA, we mutated ClyA-CS⁴¹ variant by substituting a serine with a cysteine at position 110 (ClyA-S110C, FIG. 1A). Then, a 16nt DNA oligonucleotide with an azide group at 3′ end (f-azide) was attached to ClyA-S110C by using a maleimide-PEG4-DBCO linker (FIG. 1B). With the addition of 20-fold excess of the linkers to the ClyA-S110C, the band of the product was entirely upshifted compared to ClyA-S110C in the SDS-PAGE gel, indicating a high yield of ClyA-DBCO products (FIG. 1C). Subsequently, the purified ClyA-DBCO reacted with 1.5-fold excess of f-azide, leading to a full yield of ClyA-f constructs (FIG. 1C). Furthermore, after the self-assembly in the presence of detergent to form oligomerized pores, ClyA-S110C and ClyA-f dodecamers⁴¹ (FIG. 1D, band I) were extracted from the blue native polyacrylamide gel⁴². On the basis of the high conjugation efficiency of ClyA-f monomers and homogeneity of the oligomerization, it can be assumed that on each ClyA-f dodecamer, there are approximately 12 oligos available for nanobody attachment.

To allow nanobodies to anchor on ClyA nanopores, nanobodies were produced with an azide group at N-terminal through unnatural amino acid incorporation by amber codon suppression³⁸ and conjugated to a complementary strand of oligo f containing a DBCO group (f′-DBCO) at 5′ end, via click chemistry. As a proof-of-concept, Ty1 nanobody, that can reversibly bind the receptor binding domain (RBD) of SARS-CoV-2 Spike proteins, was conjugated with f′. The binding activity of the oligo-attached nanobody was examined using the bio-layer interferometry assay, which showed that the attachment of oligos did not affect the binding affinity of Ty1 nanobody to the RBD (data not shown). Furthermore, to test the feasibility of the attachment of nanobodies to ClyA, the ClyA-f monomers were incubated with 5-fold excess of the Ty1-f′ conjugates and analyzed by SDS-polyacrylamide gel. It displayed that ClyA-f had a clear mobility shift due to nanobody attachment and suggested that the attachment efficiency was up to 100% (data not shown). Eventually, the nanobody-functionalized ClyA nanopores (ClyA-f-nb) were prepared by incubating the ClyA-f dodecamers with the respective nanobody-f modules.

### Example 2: Characterization of Nanobody-Functionalized ClyA Nanopore

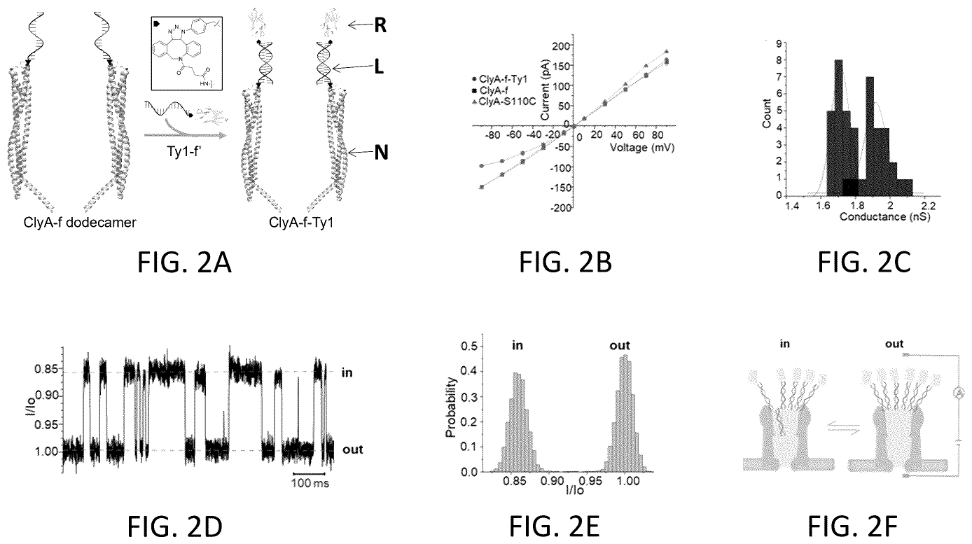

Firstly, we conducted the electrical characterization of ClyA-S110C, ClyA-f, and Ty1-modified ClyA (ClyA-f-Ty1) at different applied potentials using single-channel recording system, to investigate the effect of the attachment of ssDNA and nanobodies. At applied potentials of ±35 mV, the current traces of ClyA-f were similar to that of ClyA-S110C and no specific signal caused by the attached oligos entering the nanopore was observed (data not shown). However, the I-V curves showed that the open pore current of ClyA-f was slightly smaller than that of ClyA-S110C at positive bias ranging from 10 mV to 90 mV (FIG. 2B), which indicated that driven by the applied positive potential, the attached ssDNA entering ClyA, partially blocked the pore. Nevertheless, the conductive behavior of ClyA-f was not affected by the attachment of ssDNA at negative bias (FIG. 2B).

On contrary, the attachment of Ty1 nanobodies had no effect on the ClyA-f-Ty1 pore at a positive potential (+35 mV), whereas the pore was partially blocked compared to ClyA-f when a negative potential (−35 mV) was applied. When lowering the applied potential to −20 mV, we observed transient and reversible blockade signals (FIG. 2D). These signals are composed of two current levels (in and out), where one (out) is similar to the one expected for the open pore current, while the other is consistent with the entry of one nanobody inside the nanopore. At the same applied potential, the current blockade percentage ((Iₒ−I_{b})/Iₒ×100 (or ΔI/Iₒ×100, Iₒ is the open pore current and I_{b} is the blocked pore current) of these signals was 14.2±0.3% (n=3) and the dwell time of the blockade signals (tᵢₙ) was 21.09±1.06 ms (n=3). By fitting the all-point histogram of the current traces using the Gaussian function and calculating the proportion of the area under the curve, we found that the open probability of ClyA-f-Ty1 at −20 mV was 51% (FIG. 2E).

Moreover, by measuring the blocked pore current at different applied potentials, the I-V curve of ClyA-f-Ty1 was obtained, which demonstrated that the current of ClyA-f-Ty1 was smaller than that of the non-nanobody attached ClyA-f at negative bias ranging from −10 to −90 mV (FIG. 2B). As a result, the conductance of ClyA-f-Ty1 (1.71±0.01 nS, n=22) at −35 mV was smaller than that of ClyA-f (1.92±0.01 nS, n=22) (FIG. 2C). These results showed that the attachment of Ty1 resulted in voltage-dependent gating of ClyA nanopores.

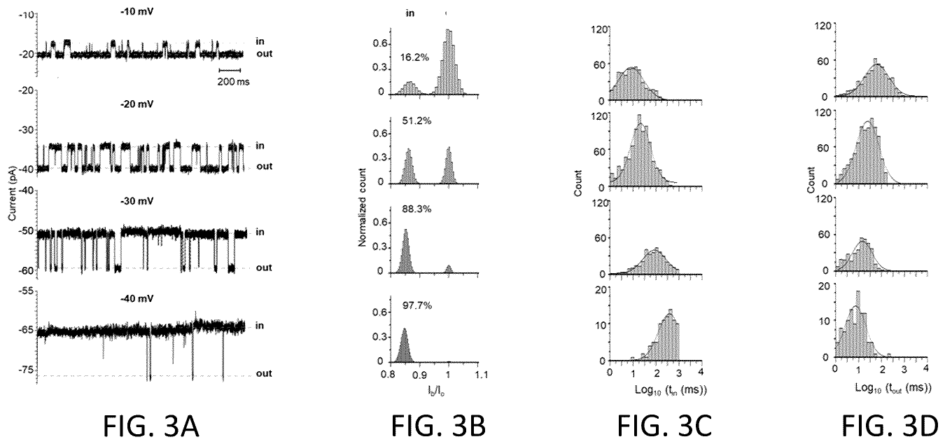

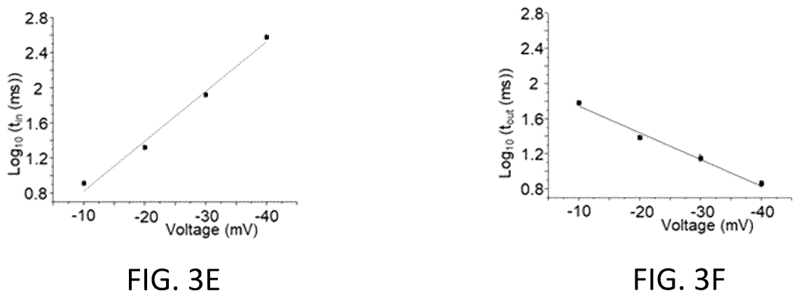

To further confirm that these blockade signals were caused by the movement of the attached nanobodies, we investigated the dependency of these blockade signals on the applied potentials. With the increase of the applied potential from −10 mV to −40 mV, the blockade probability of the pore and the dwell time of the blockade signals (tᵢₙ) remarkably increased, whereas the interval time of the pore remaining open (tₒᵤₜ) greatly decreased (FIGS. 3A-3F). For example, at a potential of −50 mV and above, for this specific nanobody the ClyA pore was almost permanently blocked. However, by reversing applied potential, ClyA-f-Ty1 nanopore can return to an unblocked state. These results indicated the blockade signals were not caused by molecule translocation as higher voltage usually drives molecules to translocate through nanopore faster (translocation would be evidenced by shorter blockade dwell times at higher voltages). It is known that ClyA-AS generates a strong electroosmotic flow⁴³, which under negative applied potentials induces the capture of a variety of proteins¹⁹. Given the small size of a nanobody (a diameter of 2.5 nm and a height of 4 nm⁴⁴) and the flexible linkage of the 16 bp DNA duplex (a length of about 5.5 nm), the current blockades are the result of coupled Ty1 nanobodies entering inside the nanopore in close proximity to the constriction region of the pore (FIG. 2F).

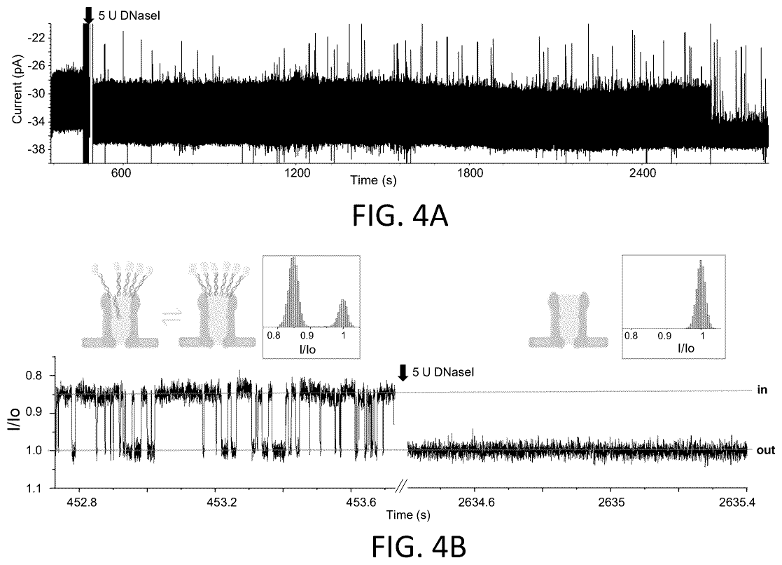

In addition, we observed irreversible opening of the pore after adding 5 U of DNase I to the first side (e.g., a cis side) of the chamber in the presence of Mg²⁺ for about 30 mins at −20 mV (FIGS. 4A-4B), as a result of cleavage of the dsDNA linker. This result confirmed that the nanobodies were successfully attached on the ClyA nanopore by DNA duplex formation and it offered additional evidence for the interpretation of the blockade signals. Herein, we use “in” and “out” to define the position of the nanobodies either inside the nanopore vestibule or outside the nanopore vestibule respectively, and tᵢₙ and tₒᵤₜ to represent the time that the nanobodies stay inside and outside of the nanopore, respectively. These results demonstrate attachment of nanobodies to the ClyA nanopore via a flexible oligonucleotide linker that enables the coupled nanobody to dynamically move in and out of the nanopore, partially blocking the ionic current when in the nanopore. Further, the results demonstrate the ability to control the dynamics between the in and the out states through applied voltage.

### Example 3: Real-Time Detection of SARS-CoV-2 Spike Protein

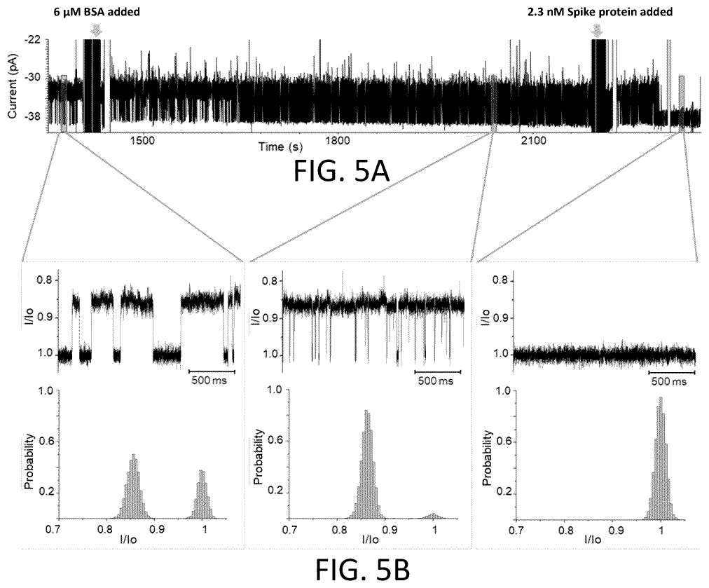

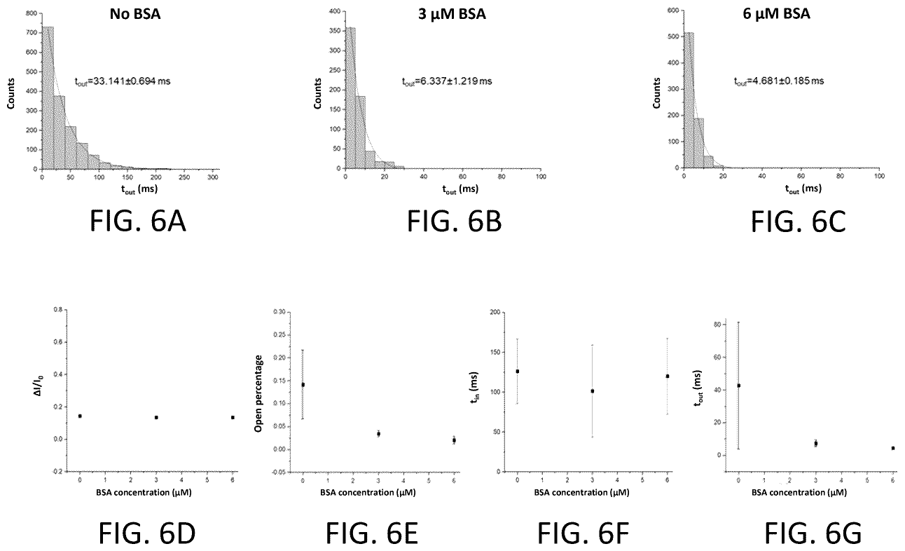

Bovine serum albumin (BSA) has been widely used as a blocking agent in sensing techniques like ELISA⁴⁵ to eliminate the nonspecific interaction such as protein-protein or protein-surface⁴⁶. In our case, with the addition of BSA to the first side (e.g., a cis side) of ClyA-f-Ty1 nanopore, no additional blockade signals caused by BSA translocation were observed. Surprisingly, we found that both tₒᵤₜ and open probability of ClyA-f-Ty1 (the probably of being in the out state) decreased with the increasing concentration of BSA (FIGS. 5A-5B, FIGS. 6A-6G). For example, in the presence of 6 μM BSA and at a bias of −20 mV, the tₒᵤₜ of ClyA-f-Ty1 decreased from 42.9±38.9 ms to 4.64±0.38 ms and the probability of ClyA-f-Ty1 being in the open-state decreased from 14.2±7.5% to 2.1±0.8%. These results suggested that the presence of BSA drastically reduced the time in which the ClyA nanopore is unoccupied by a coupled Ty1 nanobody. Given that BSA possesses a dimension of about 14×4×4 nm and a pI of 4.7 in aqueous solution⁴⁷, it was very likely BSA created a crowded environment outside of the nanopore, hence increasing the chance of nanobodies entering the pore. Similar crowding effects have been used for enhancing the capture of macromolecules in previous studies^{9 48 49}. Moreover, the addition of BSA greatly minimized the pore-to-pore variance of ClyA-f-Ty1 (FIG. 6E, FIG. 6G). Therefore, for further sensing applications, 6 μM BSA was added to the first side of the fluid chamber to minimize the background signal.

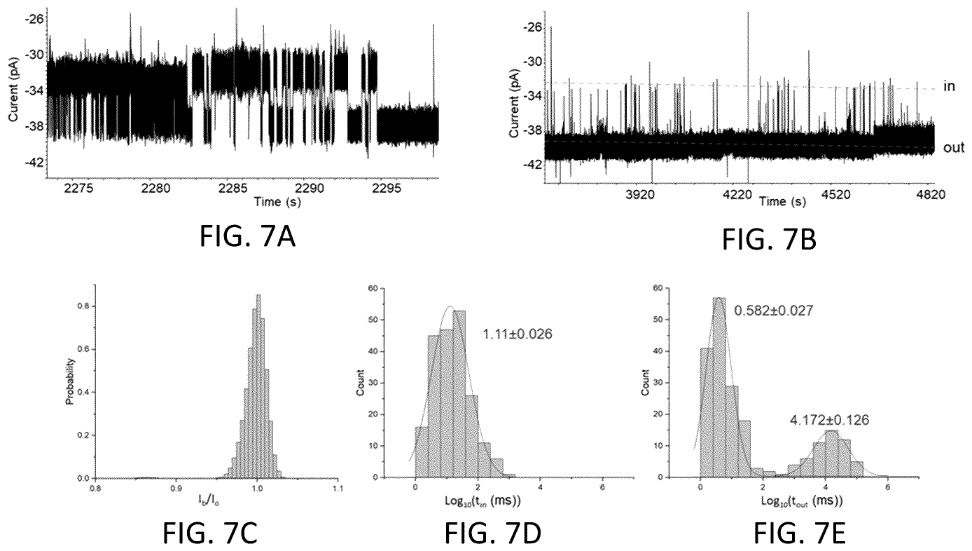

Multivalent interaction has been widely exploited to improve binding affinity and enhance sensing sensitivity^{50 51}. It was reported that the binding affinity between the SARS-CoV-2 Spike protein and Ty1 was dramatically increased by multimerization of the nanobody³⁹. Given the dodecamer structure and well-defined distance, the ClyA nanopore is predicted to be an optimal scaffold for multimerizing nanobodies in close proximity to each other so that multiple nanobodies can bind to a single protein simultaneously, to increase the sensitivity of spike protein recognition. To test the feasibility of this sensing system, SARS-CoV-2 Spike protein was added to the first side (e.g., cis side) of the fluid chamber of the ClyA-f-Ty1 nanopore system at a final concentration of 2.3 nM in the presence of 6 μM BSA. Remarkably, after about 1 min, we observed that the frequency of the blockade signals started to decrease and the tₒᵤₜ increased (FIGS. 5A-5B, FIG. 7A). Shortly thereafter the current traces became almost entirely locked into the “out” state, which was in accordance with the ClyA-f-Ty1 nanopore recovering to open pore current state (˜−38 pA) (FIGS. 5A-5B). In a recording time of about 25 minutes post addition of the Spike protein, the probability of Ty1 locating outside ClyA lumen (open probability) increased from 3.9±0.4% to 98.9±0.6% (n=3, FIGS. 7A-7E), which suggested that upon being captured by Spike, Ty1 nanobodies were retained outside of ClyA lumen. Moreover, with the presence of 2.3 nM Spike, the histogram of the logarithm of tₒᵤₜ showed two peaks (FIG. 7E) with an average interevent time of 5.03±1.34 ms and 20230.19 ±1.95 ms (n=3), respectively. The interevent duration of the first peak was very close to that of before adding Spike protein (4.48±1.32 ms, n=3), indicating those events were attributed to the unbound Ty1 nanobodies non-specifically locating inside and outside the nanopore, whereas the events of the second peak were likely caused by the binding of Spike to the nanobodies. Compared to the first peak, the time of the second peak increased by 3 orders of magnitude, which suggested that the binding interaction between the Spike trimer and the multimerized Ty1 nanobodies was very strong.

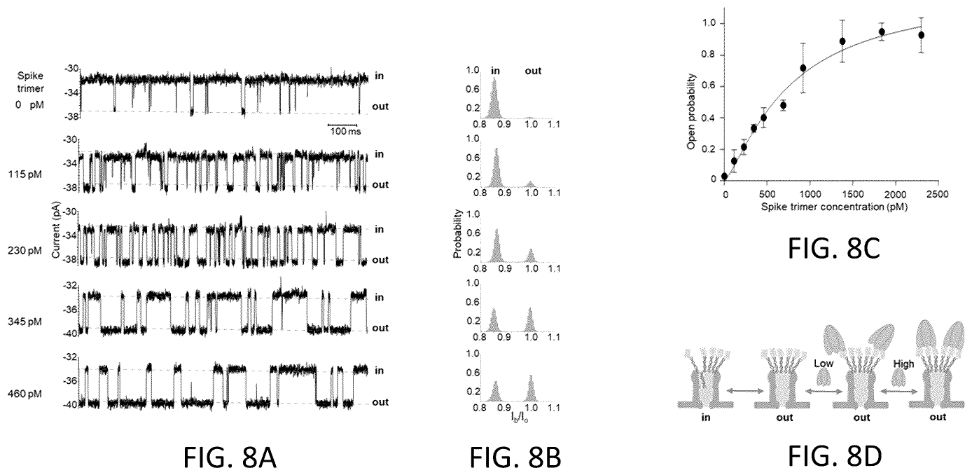

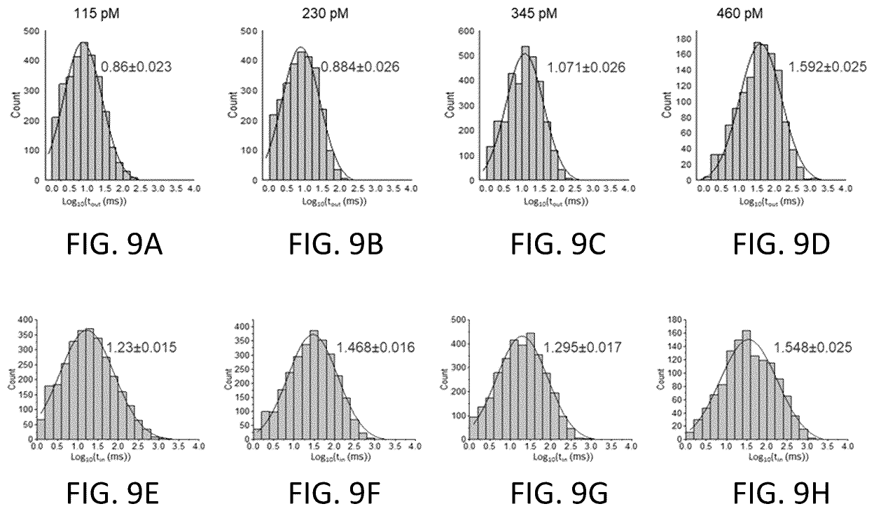

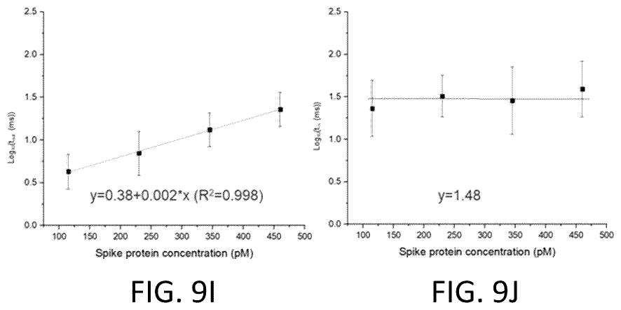

To make a calibration curve for Spike detection and to further investigate the binding kinetics of the trimeric Spike with the multimerized Ty1 nanobodies, we tested the response of ClyA-f-Ty1 nanopore to different concentrations of Spike protein. At lower concentrations (0-460 pM), the open probability of ClyA-f-Ty1 increased with increasing Spike concentration over the entire range (FIGS. 8A-8B). We also found that the time of Ty1 locating outside the pore (tₒᵤₜ) increased approximately linearly with increasing concentration of Spike protein, while the time of Ty1 lodging inside the nanopore (tᵢₙ) was independent of the concentration (from 100 to 500 pM, FIGS. 9A-9H). This confirmed that the increased open probability was indeed caused by Spike protein associating with Ty1 nanobodies. As the Spike is trimeric protein that can interact with three Ty1 nanobodies⁵², any of the 12 Ty1 on a ClyA nanopore being occupied by one Spike would modulate the ionic flow. Thus, this makes our platform capable to sensitively detect Spike at picomolar concentration. When further increasing the Spike concentration, we found that the open probability had a positive correlation with the concentration and it reached a plateau at around 2 nM (FIG. 8C). The data can be fitted by Hill-Langmuir equation with a hill coefficient greater than 1 (n=1.31), indicating that the binding between the trimeric Spike and the multimerized Ty1 was with positive cooperativity. Moreover, the previously observed long interevent duration caused by Spike binding and the small dissociation constant (K_{d}=760.6 pM) was consistent with the fact that cooperative binding between multiple ligands and the same receptor can create a much stronger binding affinity^{50 53}. Therefore, we conclude that nanopores with multiple binding ligands to the same protein have great potential for highly sensitive detection.

### Example 4: Detection of SARS-CoV-2 Spike Protein in Blood

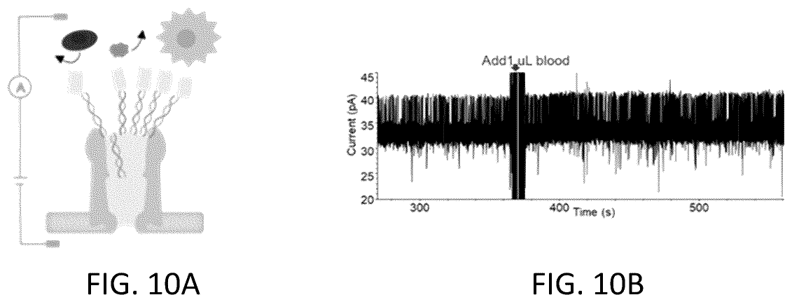

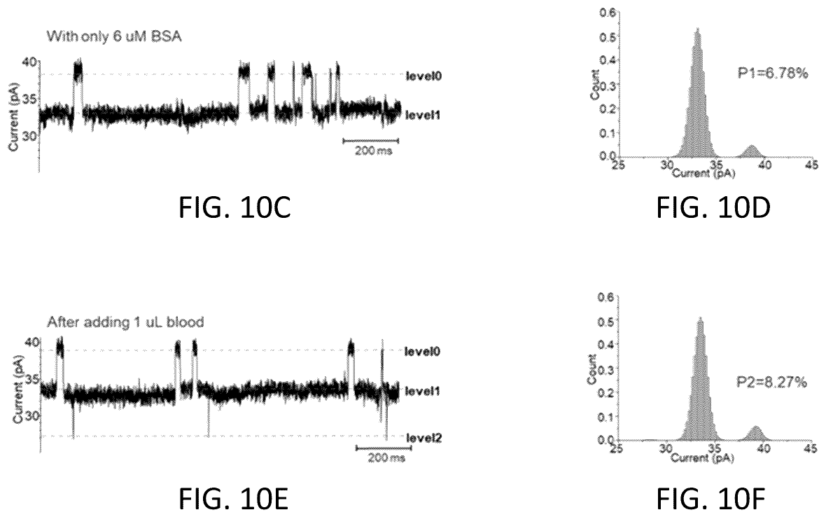

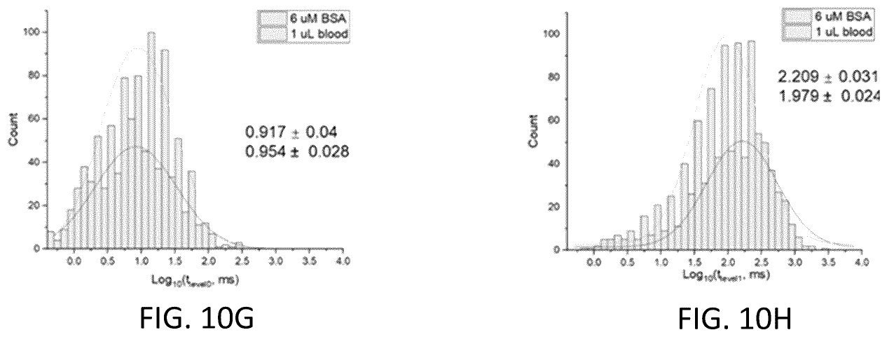

For sensing application in the clinic, it is crucial that the sensing efficiency and specificity of the sensor is not affected by blood components such as proteins, red and white blood cells and platelets. To test the influence of blood components using our ClyA-f-Ty1 pore sensor, 1 μL (final concentration: 0.2% v/v) of defibrinated sheep blood was added to the first-side (e.g., cis side) of the fluid chamber in the presence of BSA (FIG. 10A). Rewardingly, the conductive behavior of ClyA-f-Ty1 nanopore was only slightly affected by the blood and the membrane remained stable (FIGS. 10B, 10C, 10E). No obvious blood-induced blockade was observed, except for very few transient blockade signals with current blockage of around 31.5%±0.1% (FIG. 10E, level 2). However, the dwell time of those events were very short (˜0.6 ms), suggesting it might be due to transient collision by proteins or platelets in the blood. Moreover, the changes of the open probability, the dwell time and the interevent time of the ClyA-f-Ty1 nanopore before and after the addition of blood were negligible (FIGS. 10D-10H).

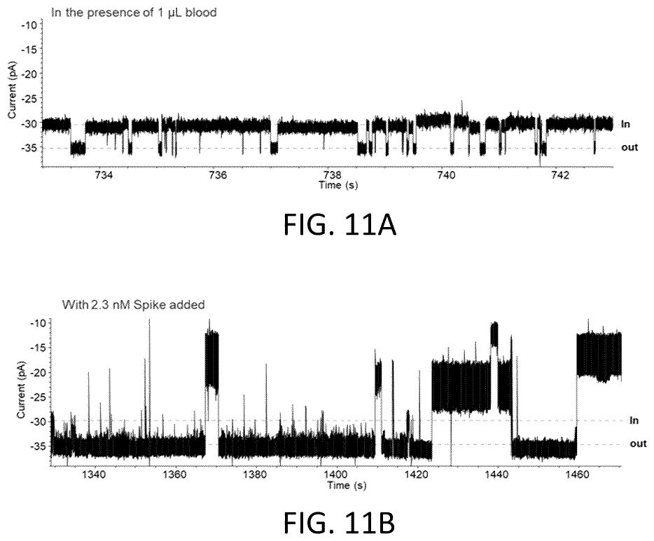

After the addition of 2.3 nM Spike, the nanopore largely transited to open state due to binding of the Ty1 nanobodies. Some large and seconds-long blockade events were also observed in this state (FIGS. 11A-11B). Likely, in the absence of Spike proteins, the steric hindrance of nanobodies on ClyA prevented blood components from entering the pore, whereas upon Spike protein binding to the nanobodies, the pore remained open so that some large proteins in blood occasionally entered the nanopore.

It is worth noting that unlike other approaches, in this system the protein does not need to enter the nanopore to be detected. This is important, because the applied potential required for protein detection in this assay was just −20 mV, which was much lower than that required for capturing proteins into nanopores²². The lower voltage reduces the chances of capturing unwanted background contaminants in the nanopore. Furthermore, the coupled nanobodies at the entrance to the nanopore further prevent capture and interference of unwanted proteins and contaminants (e.g. background proteins in blood) in the nanopore, which drastically increases the selectivity of the nanopore for the proteins.

### Example 5: General Applicability of Nanobody-Functionalized Nanopores as Protein Sensors

This example demonstrates that the concept exemplified for Spike protein detection is widely applicable to various other proteins when using the appropriate nanobody. Nanobodies have similar characteristics in sizes and shapes³⁴. It is expected, therefore, that a variety of nanobodies can provoke similar transient blockage signals when immobilized on ClyA nanopore, thus allowing the detection of variable-sized proteins. Taking advantage of the modularity of our approach, we functionalized ClyA nanopores with nanobodies 2Rs15d (ClyA-f-15d), 2Rb17c (ClyA-f-17c) and nb22 (ClyA-f-nb22), respectively. Among these nanobodies, 2Rs15d and 2Rb17c⁵⁴ recognize N-terminal half and C-terminal half of human epidermal growth factor receptor 2 (HER2) proteins that are highly expressed in breast cancer. Nanobody nb22⁵⁵ recognizes murine urokinase-type plasminogen activator (muPA) which is a biomarker associated with cancer progression.

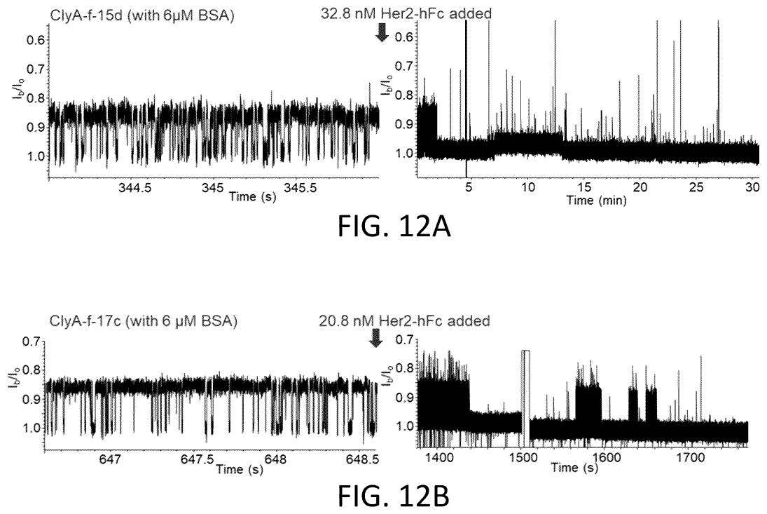

All of the three nanobodies were successfully conjugated with the oligo f′ and these nanobodies could be functionalized on ClyA with high attachment efficiency. Due to their similarity in size, shape and surface charge, we assume that these nanobodies have similar effect on the electrical behavior of ClyA as Ty1. Indeed, all of the nanobody-conjugated ClyA nanopores induced similar blockage signals at an applied potential of −20 mV (FIG. 12A-12B).

In the presence of 6 μM BSA, the blockade percentage caused by 2Rs15d, 2Rb17c, and nb22 were 11.7%±0.1%, 14.2%±0.4%, and 13.7%±0.1%, respectively. To verify the protein sensing capability, recombinant soluble protein Her2-hFc (96 kDa) was added to the ClyA-f-15d and ClyA-f-17c pores, respectively. Similar to the phenomenon observed for the interaction of Spike with ClyA-f-Ty1, both nanobody-functionalized nanopores showed significantly increased open probability after the addition of Her2-hFc as a result of the protein binding to the coupled nanobodies (FIG. 12A-12B).

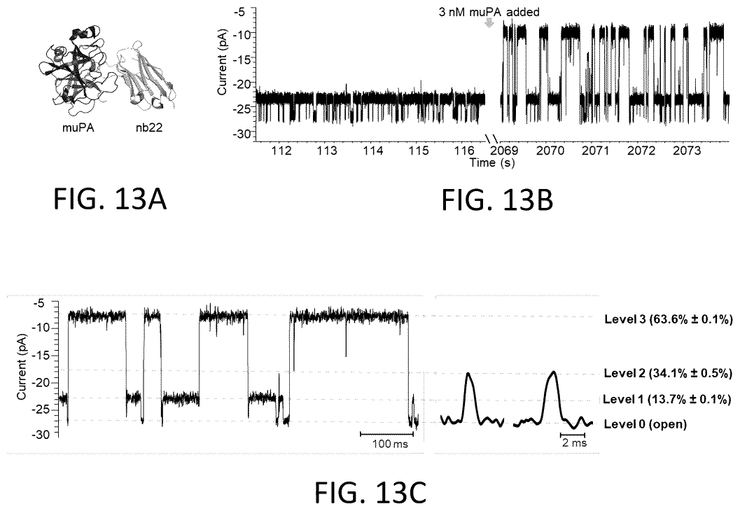

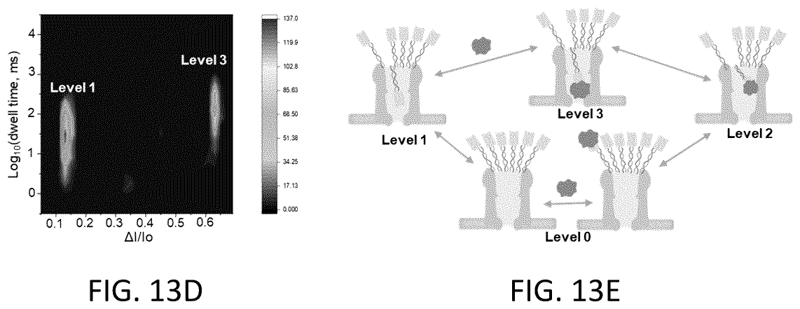

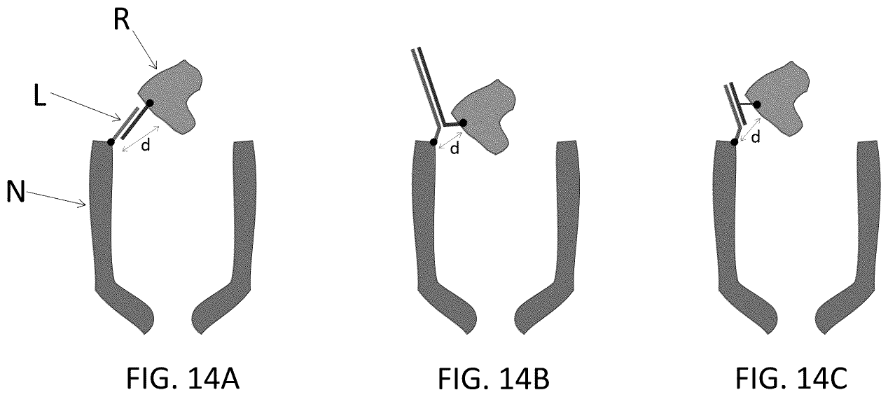

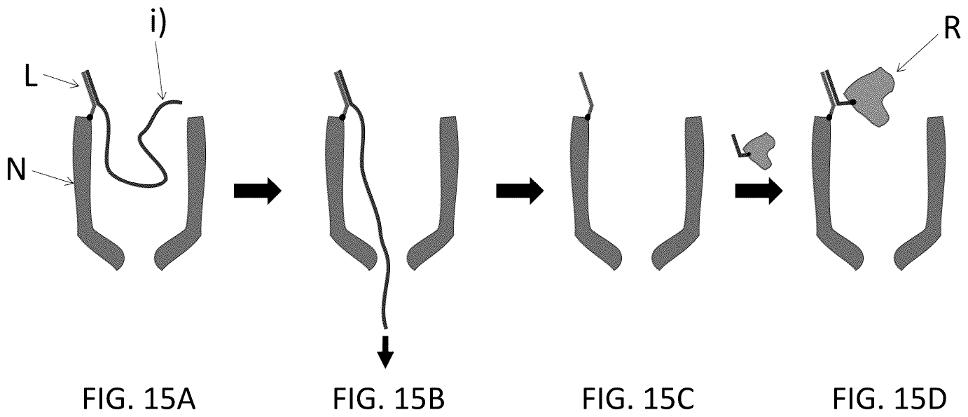

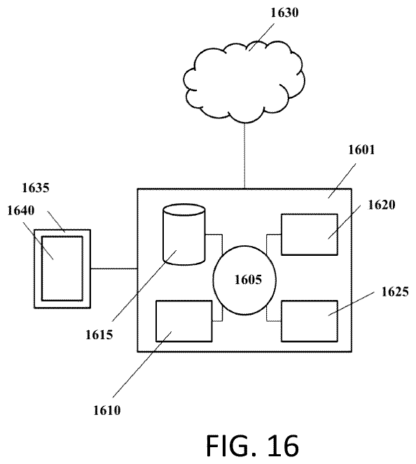

Furthermore, we tested the feasibility of ClyA-f-nb22 for protein sensing. Interestingly, after protein muPA (48 kDa, pI 8.53, FIG. 13A) was added to the first side (e.g., cis side) of ClyA-f-nb22 pores (FIG. 13B), at a potential of −15, we observed a new class of blockade events (levels 2 and 3) in addition to the open-pore level (level 0) and events provoked by the nanobody (level 1). At −15 mV the new Level 3 blockade showed a current block of 63.6±0.1% and was relatively long in duration of 45.45±1.50 ms, while Level 2 blocked to 34.1±0.5% and was very short in duration of 1.60±0.43 ms. Level 3 blockades were not observed before adding muPA (FIG. 13B left panel) or when muPA was added to ClyA-f or ClyA-f-Ty1 nanopores (data not shown), suggesting that the level 3 events were not caused by free muPA protein itself nor by non-specific interaction between nanobodies and the proteins. More likely, level 3 blockades reflect the entry of nb22: muPA complex inside the nanopore. In addition, as the applied potential increased from −5 mV to −15 mV, the dwell time of the level 3 events increased by around 1.5 orders of magnitude (data not shown), which was consistent with the fact that positively charged muPA: nb22 complex tends to reside in the nanopore for a longer time at higher negative potential. These results further confirm that muPA complexing with nb22 enters the ClyA pore, provoking the level 3 blockade events.

In comparison, Level 2 blockades did not significantly change with applied voltage. Given the short dwell time and non-voltage dependency, Level 2 blockade may reflect the transient collision of nb22: muPA complex with ClyA nanopore rather than full entry into the nanopore.

Based on the above analysis, we built a model (FIG. 13E) displaying the conformational transitions of ClyA-f-nb22 in response to muPA, which corresponded to the observed multiple current levels. Taken together these results demonstrate the ability to detect smaller analytes inside the nanopore through binding to the coupled nanobody.

### REFERENCES

1. Orit Braha et al. Simultaneous stochasticsensing of divalent metal ions. Nat. Biotechnol. 18, 1005-1007 (2000) is incorporated by reference herein in its entirety.
2. Zeng, T. et al. DNA-based detection of mercury (<scp>ii</scp>) ions through characteristic current signals in nanopores with high sensitivity and selectivity. Nanoscale 6, 8579-8584 (2014) is incorporated by reference herein in its entirety.
3. Zhang, X. et al. Real-time sensing of neurotransmitters by functionalized nanopores embedded in a single live cell. Mol. Biomed. 2, 6 (2021) is incorporated by reference herein in its entirety.
4. Boersma, A. J., Brain, K. L. & Bayley, H. Real-time stochastic detection of multiple neurotransmitters with a protein nanopore. ACS Nano 6, 5304-5308 (2012) is incorporated by reference herein in its entirety.
5. Wang, Y., Zheng, D., Tan, Q., Wang, M. X. & Gu, L. Q. Nanopore-based detection of circulating microRNAs in lung cancer patients. Nat. Nanotechnol. 6, 668-674 (2011) is incorporated by reference herein in its entirety.
6. Cao, C. et al. Discrimination of oligonucleotides of different lengths with a wild-type aerolysin nanopore. Nat. Nanotechnol. 11, 713-718 (2016) is incorporated by reference herein in its entirety.
7. Deamer, D., Akeson, M. & Branton, D. Three decades of nanopore sequencing. Nat. Biotechnol. 34, 518-524 (2016) is incorporated by reference herein in its entirety.
8. Stefureac, R., Long, Y. T., Kraatz, H. B., Howard, P. & Lee, J. S. Transport of α-helical peptides through α-hemolysin and aerolysin pores. Biochemistry 45, 9172-9179 (2006) is incorporated by reference herein in its entirety.
9. Larimi, M. G., Mayse, L. A. & Movileanu, L. Interactions of a Polypeptide with a Protein Nanopore Under Crowding Conditions. ACS Nano 13, 4469-4477 (2019) is incorporated by reference herein in its entirety.
10. Fahie, M., Chisholm, C. & Chen, M. Resolved single-molecule detection of individual species within a mixture of anti-biotin antibodies using an engineered monomeric nanopore. ACS Nano 9, 1089-1098 (2015) is incorporated by reference herein in its entirety.
11. Varongchayakul, N., Song, J., Meller, A. & Grinstaff, M. W. Single-molecule protein sensing in a nanopore: a tutorial. Chemical Society reviews vol. 47 8512-8524 (2018) is incorporated by reference herein in its entirety.
12. Waduge, P. et al. Nanopore-Based Measurements of Protein Size, Fluctuations, and Conformational Changes. ACS Nano 11, 5706-5716 (2017) is incorporated by reference herein in its entirety.
13. Yusko, E. C. et al. Real-time shape approximation and fingerprinting of single proteins using a nanopore. Nat. Nanotechnol. 12, 360-367 (2017) is incorporated by reference herein in its entirety.
14. Rotem, D., Jayasinghe, L., Salichou, M. & Bayley, H. Protein detection by nanopores equipped with aptamers. J. Am. Chem. Soc. 134, 2781-2787 (2012) is incorporated by reference herein in its entirety.
15. Rodriguez-Larrea, D. & Bayley, H. Multistep protein unfolding during nanopore translocation. Nat. Nanotechnol. 8, 288-295 (2013) is incorporated by reference herein in its entirety.
16. Waduge, P. et al. Nanopore-Based Measurements of Protein Size, Fluctuations, and Conformational Changes. ACS Nano 11, 5706-5716 (2017) is incorporated by reference herein in its entirety.
17. Van Meervelt, V. et al. Real-Time Conformational Changes and Controlled Orientation of Native Proteins Inside a Protein Nanoreactor. J. Am. Chem. Soc. 139, 18640-18646 (2017) is incorporated by reference herein in its entirety.
18. Soskine, M., Biesemans, A. & Maglia, G. Single-Molecule Analyte Recognition with ClyA Nanopores Equipped with Internal Protein Adaptors. J. Am. Chem. Soc. 137, 5793-5797 (2015) is incorporated by reference herein in its entirety.
19. Zernia, S., Van Der Heide, N. J., Galenkamp, N. S., Gouridis, G. & Maglia, G. Current Blockades of Proteins inside Nanopores for Real-Time Metabolome Analysis. ACS Nano 14, 2296-2307 (2020) is incorporated by reference herein in its entirety.
20. Jain, M. et al. Nanopore sequencing and assembly of a human genome with ultra-long reads. Nat. Biotechnol. 36, 338-345 (2018) is incorporated by reference herein in its entirety.
21. Huang, G., Willems, K., Soskine, M., Wloka, C. & Maglia, G. Electro-osmotic capture and ionic discrimination of peptide and protein biomarkers with FraC nanopores. Nat. Commun. 8, 935 (2017) is incorporated by reference herein in its entirety.
22. Van Meervelt, V., Soskine, M. & Maglia, G. Detection of two isomeric binding configurations in a protein-Aptamer complex with a biological nanopore. ACS Nano 8, 12826-12835 (2014) is incorporated by reference herein in its entirety.
23. Huang, G. et al. Electro-osmotic vortices promote the capture of folded proteins by plyab nanopores. Nano Lett. 20, 3819-3827 (2020) is incorporated by reference herein in its entirety.
24. Huang, G. et al. PlyAB Nanopores Detect Single Amino Acid Differences in Folded Haemoglobin from Blood**. Angew. Chemie Int. Ed. 61, (2022) is incorporated by reference herein in its entirety.
25. Soskine, M. et al. An Engineered ClyA Nanopore Detects Folded Target Proteins by Selective External Association and Pore Entry. Nano Lett. 12, 4895-4900 (2012) is incorporated by reference herein in its entirety.
26. Xie, H., Braha, O., Gu, L. Q., Cheley, S. & Bayley, H. Single-molecule observation of the catalytic subunit of cAMP-dependent protein kinase binding to an inhibitor peptide. Chem. Biol. 12, 109-120 (2005) is incorporated by reference herein in its entirety.
27. Wang, S., Haque, F., Rychahou, P. G., Evers, B. M. & Guo, P. Engineered nanopore of phi29 dna-packaging motor for real-time detection of single colon cancer specific antibody in serum. ACS Nano 7, 9814-9822 (2013) is incorporated by reference herein in its entirety.
28. Howorka, S., Nam, J., Bayley, H. & Kahne, D. Stochastic detection of monovalent and bivalent protein-ligand interactions. Angew. Chemie-Int. Ed. 43, 842-846 (2004) is incorporated by reference herein in its entirety.
29. Rotem, D., Jayasinghe, L., Salichou, M. & Bayley, H. Protein detection by nanopores equipped with aptamers. J. Am. Chem. Soc. 134, 2781-2787 (2012) is incorporated by reference herein in its entirety.
30. Thakur, A. K. & Movileanu, L. Real-time measurement of protein-protein interactions at single-molecule resolution using a biological nanopore. Nat. Biotechnol. 37, 96-101 (2019) is incorporated by reference herein in its entirety.
31. Muyldermans, S. Nanobodies: Natural Single-Domain Antibodies. Annu. Rev. Biochem. 82, 775-797 (2013) is incorporated by reference herein in its entirety.
32. Beghein, E. & Gettemans, J. Nanobody technology: A versatile toolkit for microscopic imaging, protein-protein interaction analysis, and protein function exploration. Front. Immunol. 8, 1-14 (2017) is incorporated by reference herein in its entirety.
33. Kang, W. et al. Nanobody Conjugates for Targeted Cancer Therapy and Imaging. Technol. Cancer Res. Treat. 20, 153303382110101 (2021) is incorporated by reference herein in its entirety.
34. Zhong, Z. et al. Positive charge in the complementarity-determining regions of synthetic nanobody prevents aggregation. Biochem. Biophys. Res. Commun. 572, 1-6 (2021) is incorporated by reference herein in its entirety.
35. Jullien, D. et al. Chromatibody, a novel non-invasive molecular tool to explore and manipulate chromatin in living cells. J. Cell Sci. 129, 2673-2683 (2016) is incorporated by reference herein in its entirety.
36. Maier, J., Traenkle, B. & Rothbauer, U. Real-time analysis of epithelial-mesenchymal transition using fluorescent single-domain antibodies. Sci. Rep. 5, 13402 (2015) is incorporated by reference herein in its entirety.
37. Van Audenhove, I. et al. Mapping cytoskeletal protein function in cells by means of nanobodies. Cytoskeleton 70, 604-622 (2013) is incorporated by reference herein in its entirety.
38. Teodori, L. et al. Site-specific nanobody-oligonucleotide conjugation for super-resolution imaging. J. Biol. Methods 9, e159 (2022) is incorporated by reference herein in its entirety.
39. Moliner-Morro, A. et al. Picomolar SARS-CoV-2 Neutralization Using Multi-Arm PEG Nanobody Constructs. Biomolecules 10, 1661 (2020) is incorporated by reference herein in its entirety.
40. Bao, K. et al. Nanobody multimerization strategy to enhance the sensitivity of competitive ELISA for detection of ochratoxin A in coffee samples. Food Control 127, 108167 (2021) is incorporated by reference herein in its entirety.
41. Soskine, M., Biesemans, A., De Maeyer, M. & Maglia, G. Tuning the Size and Properties of ClyA Nanopores Assisted by Directed Evolution. J. Am. Chem. Soc. 135, 13456-13463 (2013) is incorporated by reference herein in its entirety.
42. Soskine, M. et al. An engineered ClyA nanopore detects folded target proteins by selective external association and pore entry. Nano Lett. 12, 4895-4900 (2012) is incorporated by reference herein in its entirety.
43. Willems, K. et al. Accurate modeling of a biological nanopore with an extended continuum framework. Nanoscale 12, 16775-16795 (2020) is incorporated by reference herein in its entirety.
44. Kijanka, M., Dorresteijn, B., Oliveira, S. & van Bergen en Henegouwen, P. M. Nanobody-based cancer therapy of solid tumors. Nanomedicine 10, 161-174 (2015) is incorporated by reference herein in its entirety.
45. Xiao, Y. & Isaacs, S. N. Enzyme-linked immunosorbent assay (ELISA) and blocking with bovine serum albumin (BSA)—not all BSAs are alike. J. Immunol. Methods 384, 148-151 (2012) is incorporated by reference herein in its entirety.
46. Lee, J. et al. BSA as additive: A simple strategy for practical applications of PNA in bioanalysis. Biosens. Bioelectron. 69, 167-173 (2015) is incorporated by reference herein in its entirety.
47. Wright, A. K. & Thompson, M. R. Hydrodynamic structure of bovine serum albumin determined by transient electric birefringence. Biophys. J. 15, 137-141 (1975) is incorporated by reference herein in its entirety.
48. Yao, F. et al. Crowding-Induced DNA Translocation through a Protein Nanopore. Anal. Chem. 92, 3827-3833 (2020) is incorporated by reference herein in its entirety.
49. Chau, C. C., Radford, S. E., Hewitt, E. W. & Actis, P. Macromolecular Crowding Enhances the Detection of DNA and Proteins by a Solid-State Nanopore. Nano Lett. 20, 5553-5561 (2020) is incorporated by reference herein in its entirety.
50. Valero, J. et al. A serum-stable RNA aptamer specific for SARS-CoV-2 neutralizes viral entry. Proc. Natl. Acad. Sci. 118, e2112942118 (2021) is incorporated by reference herein in its entirety.
51. Koenig, P.-A. et al. Structure-guided multivalent nanobodies block SARS-CoV-2 infection and suppress mutational escape. Science (80-.). 371, (2021) is incorporated by reference herein in its entirety.
52. Hanke, L. et al. An alpaca nanobody neutralizes SARS-CoV-2 by blocking receptor interaction. Nat. Commun. 11, 4420 (2020) is incorporated by reference herein in its entirety.
53. Zumbro, E. & Alexander-Katz, A. Influence of Binding Site Affinity Patterns on Binding of Multivalent Polymers. ACS Omega 5, 10774-10781 (2020) is incorporated by reference herein in its entirety.
54. Vaneycken, I. et al. Preclinical screening of anti-HER2 nanobodies for molecular imaging of breast cancer. FASEB J. 25, 2433-2446 (2011) is incorporated by reference herein in its entirety.
55. Kromann-Hansen, T. et al. Discovery of a novel conformational equilibrium in urokinase-type plasminogen activator. Sci. Rep. 7, 1-11 (2017) is incorporated by reference herein in its entirety.

56. Lange, E. L. A Camelid Antibody Fragment Acting as an Orthosteric Inhibitor of Murine Urokinase-type Plasminogen Activator. (Aarhus University, 2015) is incorporated by reference herein in its entirety.
57. Richardson, M. B. et al. Synthesis and Explosion Hazards of 4-Azido-L-phenylalanine. J. Org. Chem. 83, 4525-4536 (2018) is incorporated by reference herein in its entirety.
58. Maglia, G., Heron, A. J., Stoddart, D., Japrung, D. & Bayley, H. Analysis of Single Nucleic Acid Molecules with Protein Nanopores. in Bone vol. 23 591-623 (2010) is incorporated by reference herein in its entirety.

What is claimed is:

1.-138. (canceled)

139. A method comprising:
(a) providing a nanopore system, wherein the nanopore system comprises (1) a fluid chamber and (2) a membrane comprising a nanopore, wherein the membrane separates the fluid chamber into a first side and a second side, wherein the nanopore is coupled to a protein recognition element, wherein the protein recognition element is configured to move between an internal region of the nanopore and an external region of the nanopore; and
(b) contacting the protein recognition element with an analyte.

140. The method of claim 139, wherein the protein recognition element is coupled to the nanopore via a linker.

141. The method of claim 139, wherein the protein recognition element is between about 5 kilodaltons to about 50 kilodaltons

142. The method of claim 139, wherein the protein recognition element comprises a nanobody, a Fab fragment, a single-chain variable fragment (scFv), an antibody, a monobody, an affimer, an affibody, an Adnectin, a designed ankyrin repeat protein (DARPin), an anticalin, or any combination thereof.

143. The method of claim 139, wherein the protein recognition element couples to the analyte.

144. The method of claim 143, wherein the protein recognition element coupled to the analyte effects movement of the protein recognition element.

145. The method of claim 144, wherein effecting the movement of the protein recognition element generates a change in (i) a frequency of the movement of the protein recognition element or (ii) a noise or a magnitude of a current of the nanopore system.

146. The method of claim 139, wherein the analyte comprises a protein, a peptide, a small molecule, a protein assembly, a protein/DNA assembly, a protein/RNA assembly, a steroid, a lipid, a lipid membrane, a lipid particle, a bacterium, a viral capsid, a viral particle, a cell, a dendrimer, a polymer, or any combination thereof.

147. The method of claim 139, wherein the protein recognition element is coupled to at least one subunit of the nanopore via a linker.

148. The method of claim 147, wherein the at least one subunit of the nanopore comprises a monomer of a pore-forming protein.

149. The method of claim 148, wherein the pore-forming protein comprises cytolysin A (ClyA), pleurotolysin (PlyAB), YaxAB, perforin-2, tripartite alpha-pore forming toxin, secretin, Helicobacter pylori OMC, SpoIIIAG, Gasdermin-A3, or any combination thereof.

150. The method of claim 148, wherein the pore-forming protein comprises one or more mutations.

151. The method of claim 139, wherein the nanopore is coupled to another protein recognition element.

152. The method of claim 139, wherein the nanopore system further comprises a pair of electrodes configured to generate an electrical potential across the nanopore.

153. The method of claim 152, wherein movement of the protein recognition element between an internal region of the nanopore and an external region of the nanopore effects a change in a current of the nanopore system.

154. A system comprising: (a) a fluid chamber; and (b) a membrane comprising a nanopore, wherein the membrane separates the fluid chamber into (1) a first side and (2) a second side, wherein the nanopore is coupled to a protein recognition element, wherein the protein recognition element is configured to move between an internal region of the nanopore and an external region of the nanopore.

155. The system of claim 154, wherein the protein recognition element is coupled to the nanopore via a linker.

156. The system of claim 154, wherein the protein recognition element is between about 5 kilodaltons to about 50 kilodaltons.

157. The system of claim 154, wherein the protein recognition element comprises a nanobody, a Fab fragment, a single-chain variable fragment (scFv), an antibody, a monobody, an affimer, an affibody, an Adnectin, a designed ankyrin repeat protein (DARPin), an anticalin, or any combination thereof.

158. The system of claim 154, wherein the protein recognition element is configured to couple to an analyte.


SEQUENCE LISTING (3 sequences)

SEQ ID NO: 1 - protein, 312 aa, synthetic construct
MTGIFAEQTVEVVKSAIETADGALDLYNKYLDQVIPWKTFDETIKELSRFKQEYSQEASVLVGDIKVLLMDSQDKYFEATQTVYEWAGVVTQLLSAYIQLFDGYNEKKASAQKDILIRILDDGVKKLNEAQKSLLTSSQSFNNASGKLLALDSQLTNDFSEKSSYYQSQVDRIRKEAYAGAAAGIVAGPFGLIISYSIAAGVVEGKLIPELNNRLKTVQNFFTSLSATVKQANKDIDAAKLKLATEIAAIGEIKTETETTRFYVDYDDLMLSLLKGAAKKMINTSNEYQQRHGRKTLFEVPDVGSSYHHHHH

SEQ ID NO: 2 - DNA, 16 bp, synthetic construct
  source 1..16: mol_type = other DNA
  misc_feature 1: note = linked to NH2-C6
atccgcgggtgtcggg

SEQ ID NO: 3 - DNA, 16 bp, synthetic construct
  source 1..16: mol_type = other DNA
  misc_feature 1: note = linked to NH2-C6
cccgacacccgcggat